(19) United States
(12) Patent Application Publication
(10) Pub. No.: US 2025/0138018 A1
(43) Pub. Date: May 1, 2025

(54) T CELL AND NK CELL ENGAGERS

(71) Applicant: NextPoint Therapeutics, Inc., Cambridge, MA (US)

(72) Inventors: Bijan Etemad-Gilbertson, Jamaica Plain, MA (US); Matthew Rausch, Natick, MA (US)

(21) Appl. No.: 18/836,996

(22) PCT Filed: Feb. 10, 2023
(86) PCT No.: PCT/US23/12836

§ 371 Date: Aug. 8, 2024

Related U.S. Application Data
(60) Provisional application No. 63/308,800, filed on Feb. 10, 2022.

(51) Int. Cl. G01N 33/574; C07K 16/28; C07K 16/30

(52) U.S. Cl. CPC G01N 33/57492; C07K 16/2818; C07K 16/3076; C07K 2317/31; G01N 2333/70521; G01N 2333/82

(57) ABSTRACT

The present disclosure pertains to T cell and/or NK cell engaging agents, and uses thereof. In some embodiments, the present disclosure provides bispecific antibody molecules or antigen-binding fragments thereof comprising (i) a first antigen-binding domain that selectively binds to transmembrane and immunoglobulin domain containing 2 (TMIGD2); and (ii) a second antigen-binding domain that selectively binds to at least one tumor-associated antigen (TAA). Such agents disclosed herein (e.g., bispecific antibody molecules or antigen-binding fragments thereof) can be used in methods of treating a subject having a disease, disorder, or condition and/or modulating an immune response in a subject.

### CROSS REFERENCE TO RELATED APPLICATIONS

This application claims priority to U.S. Provisional Patent Application No. 63/308,800, filed on Feb. 10, 2022, the contents of which are incorporated herein by reference in their entirety.

### BACKGROUND

Although immunotherapies have been investigated for many diseases and disorders, including cancer, functional limitations have been encountered that still need to be addressed. A network of costimulatory and co-inhibitory ligands and receptors tightly controls the immune system. Immune checkpoints negatively regulate immune response progression based on complex interactions. Currently available immunotherapies can modulate immune responses in some patients, but immune checkpoint expression and interactions with natural binding partners can vary between patients.

Therefore, a need exists for the development of new and improved therapeutic modalities optimized to target tumor sites, particularly in the tumor microenvironment.

### SUMMARY

The present disclosure, among other things, provides bispecific antibody molecules or antigen-binding fragments thereof that interact with particular targets and methods of using such bispecific antibody molecules or antigen-binding fragments thereof. In some embodiments, a bispecific antibody molecule or antigen-binding fragment thereof described herein comprises a first antigen-binding domain that selectively binds to transmembrane and immunoglobulin domain containing 2 (TMIGD2). In some embodiments, a bispecific antibody molecule or antigen-binding fragment thereof described herein comprises a second antigen-binding domain that selectively binds to at least one tumor-associated antigen (TAA). HERV-H LTR-Associating 2 (HHLA2) is an exemplary TAA, which is a B7 gene family member that is broadly expressed in a variety of tumors and antigen presenting cells. HHLA2 interacts with both inhibitory and stimulatory receptors to regulate T cell and NK cell functions. TMIGD2 is an activating receptor for HHLA2 expressed on both T cells and NK cells that interacts with HHLA2 to co-stimulate T cell and NK cell activation.

While various TAA binding agents have been investigated as immunotherapies, the present disclosure encompasses, inter alia, the discovery of bispecific antibody molecules or antigen-binding fragments thereof with novel activity that can selectively bind to at least one TAA expressed on cancer cells and selectively bind to TMIGD2 expressed on immune effector cells, particularly T cells (e.g., naïve and/or effector T cells) and/or NK cells. Combining such a tumor-directed approach to augment immune modulation in patients can provide a potent strategy to generate and shape an immune response at tumor sites, particularly in a tumor microenvironment (TME). Bispecific antibody molecules or antigen-binding fragments thereof described herein may activate both innate and adaptive immune systems. Accordingly, the present disclosure provides several examples of bispecific antibody molecules or antigen-binding fragments thereof that are particularly useful for treating a variety of cancers, including solid tumors or hematological tumors, as well as modulating an immune response in a subject.

In one aspect, the disclosure provides bispecific antibody molecules or antigen-binding fragments thereof comprising: (i) a first antigen-binding domain that selectively binds to TMIGD2; and (ii) a second antigen-binding domain that selectively binds to at least one TAA. In some embodiments, a first antigen-binding domain activates immune effector cells. In some embodiments, a first antigen-binding domain recruits immune effector cells to a tumor microenvironment. In some embodiments, immune effector cells comprise or are T cells (e.g., naïve and/or effector T cells) and/or NK cells. In some embodiments, at least one TAA comprises or is HHLA2, CD123, B7-H3, B7-H4, CD33, CD30, HER2, CD22, CD79b, Nectin-4, Trop-2, BCMA, CD19, folate receptor a, EGFR, or combinations thereof. In certain embodiments, at least one TAA comprises or is HHLA2. In certain embodiments, at least one TAA comprises or is CD123. In certain embodiments, at least one TAA comprises or is B7-H3. In certain embodiments, at least one TAA comprises or is B7-H4. In certain embodiments, at least one TAA comprises or is CD33. In certain embodiments, at least one TAA comprises or is CD30. In certain embodiments, at least one TAA comprises or is HER2. In certain embodiments, at least one TAA comprises or is CD22. In certain embodiments, at least one TAA comprises or is CD79b. In certain embodiments, at least one TAA comprises or is Nectin-4. In certain embodiments, at least one TAA comprises or is Trop-2. In certain embodiments, at least one TAA comprises or is BCMA. In certain embodiments, at least one TAA comprises or is CD19. In certain embodiments, at least one TAA comprises or is folate receptor a. In certain embodiments, at least one TAA comprises or is EGFR.

In some embodiments, a first antigen-binding domain comprises or is an antibody or an antigen-binding fragment thereof. In some embodiments, a second antigen-binding domain comprises or is an antibody or an antigen-binding fragment thereof. In some embodiments, an antigen-binding fragment comprises or is an scFv, Fab, Fab′, F(ab′)₂, nanobody, or camelid antibody. In some embodiments, an antibody or antigen-binding fragment thereof is or comprises: (i) a chimeric, human, or humanized antibody or antigen-binding fragment thereof; and/or (iii) a monoclonal antibody or antigen-binding fragment thereof. In some embodiments, an antibody or antigen-binding fragment thereof is or comprises: (i) a heavy chain constant region chosen from IgG1, IgG2, IgG3, or IgG4, and/or (ii) a light chain constant region chosen from light chain constant regions of kappa or lambda.

In some embodiments, an antibody or antigen-binding fragment comprises an Fc region with reduced effector function. In some embodiments, an Fc region comprises one or more amino acid substitutions of L234A and/or L235A, numbering according to EU index. In some embodiments, a modification comprises N297A, numbering according to EU index. In some embodiments, a first antigen-binding domain comprises or is a scFv and a second antigen-binding domain comprises or is an antibody. In some embodiments, a scFv and an antibody are covalently linked by a peptide linker. In some embodiments, a peptide linker is greater than at least five amino acids in length.

In another aspect, the disclosure provides pharmaceutical compositions comprising at least one bispecific antibody molecule or antigen-binding fragment thereof of any aspect or embodiment described herein, and a pharmaceutically acceptable carrier.

In another aspect, the disclosure provides methods of treating a subject having a disease, disorder, or condition comprising administering a therapeutically effective amount of a pharmaceutical composition of any aspect or embodiment described herein. In another aspect, the disclosure provides methods of modulating an immune response in a subject comprising administering a therapeutically effective amount of a pharmaceutical composition of any aspect or embodiment described herein.

In some embodiments, a subject has or is at risk of developing a cancer. In some embodiments, a subject has a solid tumor or a hematological cancer. In some embodiments, a solid tumor is or comprises one or more of: a renal cancer, a bone cancer, a skin cancer, a breast cancer, a cervical cancer, a colorectal cancer, an endometrial cancer, a lung cancer, an ovarian cancer, a liver cancer, cholangiocarcinoma, or a thyroid cancer. In some embodiments, a hematological cancer comprises or is a leukemia or lymphoma. In some embodiments, a leukemia comprises or is acute lymphocytic leukemia, acute myeloid leukemia, chronic myeloid leukemia, acute lymphoblastic leukemia, chronic lymphocytic leukemia, chronic leukemia, or acute leukemia. In some embodiments, a lymphoma comprises or is Hodgkin lymphoma (HL), non-Hodgkin's lymphoma, lymphocytic lymphoma, or diffuse large B cell lymphoma (DLBCL).

In some embodiments, at least one bispecific antibody molecule or antigen-binding fragment thereof of any aspect or embodiment described herein is administered in combination with at least one additional agent.

In another aspect, the disclosure provides nucleic acid molecules encoding at least one bispecific antibody molecule or antigen-binding fragment thereof of any aspect or embodiment described herein (e.g., one or more nucleic acid molecules in Tables 1-5).

In another aspect, the disclosure provides expression vectors comprising a nucleic acid molecule of any aspect or embodiment described herein.

In another aspect, the disclosure provides host cells comprising or expressing at least one bispecific antibody molecule or antigen-binding fragment thereof of any aspect or embodiment described herein, a nucleic acid molecule of any aspect or embodiment described herein, or an expression vector of any aspect or embodiment described herein.

In another aspect, the disclosure provides methods of making at least one bispecific antibody molecule or antigen-binding fragment thereof, comprising: (i) culturing a host cell comprising a nucleic acid molecule of any aspect or embodiment described herein or an expression vector of any aspect or embodiment described herein under conditions suitable for expression of at least one bispecific antibody molecule or antigen-binding fragment thereof, and (ii) recovering at least one bispecific antibody molecule or antigen-binding fragment thereof.

In another aspect, the disclosure provides methods of detecting a presence or level of a TMIGD2 polypeptide and/or a TAA in a sample comprising detecting a TMIGD2 polypeptide and/or a TAA in a sample using at least one bispecific antibody molecule or antigen-binding fragment thereof of any aspect or embodiment described herein.

In another aspect, the disclosure provides kits comprising at least one bispecific antibody molecule or antigen-binding fragment thereof of any aspect or embodiment described herein, and instructions for use.

### BRIEF DESCRIPTION OF THE DRAWING

The Figures described below, which together make up the Drawing, are for illustration purposes only, not for limitation.

FIG. 1 is a schematic showing an exemplary format of a bispecific antibody molecule using a scFv targeting TMIGD2 attached to a C-terminus of a heavy chain of an IgG antibody targeting a TAA. Linkers within the scFv domain (between V_{H} and V_{L} domains) and between scFv and Fc region are both (G₄S)₄ repeats.

FIGS. 2A-2B are graphs showing fold change in T cell activation over anti-CD3 stimulation alone of plate bound (FIG. 2A) and soluble (FIG. 2B) anti-TMIGD2/anti-CD20 bispecific antibody molecule (TankeC4S2), anti-TMIGD2 IgG4 (Amp4.5), or human IgG4 isotype control in TMIGD2-Jurkat NFAT reporter cells.

FIG. 3 is a graph showing fold change in T cell activation over anti-CD3 stimulation alone of anti-TMIGD2/anti-CD20 bispecific antibody molecule (Tanke) in combination with anti-CD3 or human IgG4 isotype control in an NFAT TMIGD2-jurkat reporter assay in the presence of TAA (CD20)-expressing Raji target cells.

FIG. 4 is a graph showing fold change in T cell activation over anti-CD3 stimulation alone of anti-TMIGD2/anti-CD20 bispecific antibody molecules (TK20-17C7-01 and TK20-39C9-2134) in combination with anti-CD3 or anti-CD20 (IgG1 LALA) control in an NFAT TMIGD2-jurkat reporter assay in the presence of TAA (CD20)-expressing Raji target cells.

### DEFINITIONS

In order for the present disclosure to be more readily understood, certain terms are first defined below. Additional definitions for the following terms and other terms are set forth throughout the specification. The publications and other reference materials referenced herein to describe the background of the disclosure and to provide additional detail regarding its practice are hereby incorporated by reference in their entirety.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) where ranges are provided, endpoints are included.

About: As used herein, the term “about” as applied to one or more values of interest, refers to a value that is similar to a stated reference value. In some embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Affinity matured: As used herein, the term “affinity matured” refers to an antibody molecule with one or more alterations in one or more CDRs thereof, which result in an improvement in affinity of an antibody molecule for an antigen, compared to a parent antibody molecule that does not possess those one or more alterations. In some embodiments, affinity matured antibody molecules will have nanomolar or even picomolar affinities for a target antigen. Affinity matured antibody molecules may be produced by any of a variety of procedures known in the art. Affinity maturation by V_{H} and V_{L} domain shuffling is described in Marks et al., BioTechnology 10:779-783 (1992). Random mutagenesis of CDR and/or framework residues is described in: Barbas et al. Proc. Nat. Acad. Sci. U.S.A 91:3809-3813 (1994); Schier et al., Gene 169:147-155 (1995); Yelton et al., J. Immunol. 155:1994-2004 (1995); Jackson et al., J. Immunol. 154 (7): 3310-9 (1995); and Hawkins et al., J. Mol. Biol. 226:889-896 (1992).

Agent: As used herein, the term “agent” refers to a form of treatment, for example, a biological, chemical, or other treatment. For example, a biological agent may be or comprise a biological entity and/or compound including, for example, an antibody molecule or antigen-binding fragment thereof, an organic molecule (e.g., a small molecule), a peptide (e.g., a fusion protein), an aptamer, a nucleic acid, a chimeric antigen receptor, a glycoprotein, a saccharide, a lipid, a growth factor, an enzyme, a synthetic molecule, a carbohydrate, a lipid, a hormone, a polymer, or a derivative, variation, complex, or any combination thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product. In some instances, again as will be clear from context, the term may be used to refer to one or more entities that is man-made in that it is designed, engineered, and/or produced through human action and/or is not found in nature. In some embodiments, an agent may be utilized in isolated or pure form. In some embodiments, an agent may be utilized in crude form. In some embodiments, agents are provided as collections or libraries, which may be screened to identify or characterize active agents within them. An agent may bind any cell moiety, such as a receptor, an antigenic determinant, or other binding site present on a target or target cell. Various agents are useful in the compositions and methods described herein.

For example, an agent can be or comprise one or more of: a chemotherapeutic agent, a hormone therapy, a cell-based therapy, treatment with hyperthermia, a photodynamic therapy, surgery, radiation, or a transplant. In some embodiments, a chemotherapeutic agent comprises or is one or more anthracyclines, one or more cytoskeletal disruptors (e.g. microtubule targeting agents such as taxanes, maytansine, and analogs thereof), one or more epothilones, one or more histone deacetylase inhibitors (HDACs), one or more topoisomerase inhibitors (e.g., one or more of inhibitors of topoisomerase I or topoisomerase II), one or more kinase inhibitors, one or more nucleotide analogs or nucleotide precursor analogs, one or more peptide antibiotics, one or more platinum-based agents, one or more retinoids, one or more vinca alkaloids, or a combination thereof. In some embodiments, a chemotherapeutic agent comprises or is one or more of: Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fludarabine, Fluorouracil, Gemcitabine, Hydroxyurea, Idarubicin, Imatinib, Irinotecan, Maytansine and/or analogs thereof (e.g. DM1), Mechlorethamine, Mercaptopurine, Methotrexate, Mitoxantrone, a Maytansinoid, Oxaliplatin, Paclitaxel, Pemetrexed, Rituxan, Teniposide, Tioguanine, Topotecan, Valrubicin, Vinblastine, Vincristine, Vindesine, or Vinorelbine. In some embodiments, a chemotherapeutic agent comprises or is an antibody-drug conjugate (ADC). In some embodiments, an ADC comprises or is hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, lorvotuzumab mertansine, or a combination thereof.

In some embodiments, a hormone therapy may be or comprise tamoxifen, raloxifene, leuprolide, bicaluatmide, granisetron, flutamide, or a combination thereof. In some embodiments, a cell-based therapy comprises or is chimeric antigen receptor T (CAR-T) cells, TCR-transduced T cells, dendritic cells, tumor infiltrating lymphocytes (TILs), natural killer (NK) cells, or a combination thereof. In some embodiments, treatment with hyperthermia comprises or is local hyperthermia (e.g., external, intraluminal, or interstitial hyperthermia), regional hyperthermia (e.g., deep tissue hyperthermia, regional perfusion, or (continuous hyperthermic peritoneal perfusion), or whole-body hyperthermia. In some embodiments, a photodynamic therapy comprises or is administration of photosensitizers, such as hematoporphyrin and its derivatives, Verteporfin (BPD-MA), phthalocyanine, photosensitizer Pc4, demethoxy-hypocrellin A, 2BA-2-DMHA, or a combination thereof. In some embodiments, surgery comprises or is surgery to remove cancerous or precancerous tissue. In some embodiments, a transplant comprises or is a stem cell transplant or an organ transplant.

Antibody Molecule: As used herein, the term “antibody molecule” refers to a polypeptide, e.g., an immunoglobulin chain or fragment thereof, including canonical immunoglobulin sequence elements sufficient to confer specific binding to a particular target, e.g., an antigen. The term “antibody molecule” encompasses antibodies and antibody fragments. As is known in the art, intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long): an amino-terminal variable (V_{H}) domain followed by three constant domains: CH1, CH2, and a carboxy-terminal CH3 (located at the base of the Y's stem). A short region, known as a “switch,” connects the heavy chain variable and constant regions. A “hinge” region connects CH2 and CH3 domains to CH1 domain. Two disulfide bonds in this hinge region connect two heavy chain polypeptides to one another in an intact antibody molecule. Each light chain comprises two domains: an amino-terminal variable (V_{L}) domain, followed by a carboxy-terminal constant (CL) domain, separated from one another by another “switch” region. Naturally produced antibodies are glycosylated, typically on a CH2 domain. Each domain in an antibody has a structure characterized by an “immunoglobulin fold” formed from two beta sheets (e.g., 3-stranded sheets, 4-stranded sheets, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, FR regions form beta sheets that provide a structural framework for the domains, and CDR loop regions from both heavy and light chains are brought together in three-dimensional space to create a single hypervariable antigen binding site located at the tip of the Y structure. An Fc region of naturally occurring antibodies binds to receptors on effector cells, including, for example, effector cells that mediate cytotoxicity. Affinity and/or other binding attributes of Fc regions for Fc receptors can be modulated through glycosylation or other modification.

In some embodiments, any polypeptide or complex of polypeptides that includes sufficient immunoglobulin domain sequences can be referred to and/or used as an “antibody molecule,” whether such polypeptide is naturally produced (e.g., generated by an organism reacting to an antigen) or produced by recombinant engineering, chemical synthesis, or other artificial system or methodology. In some embodiments, an antibody molecule is monoclonal. In some embodiments, an antibody molecule is polyclonal. In some embodiments, an antibody molecule has constant region sequences characteristic of mouse, rabbit, primate, or human antibodies. In some embodiments, one or more antibody molecule sequences are humanized, primatized, or chimeric as is known in the art. Moreover, the term “antibody molecule,” can refer to any of the art-known or developed constructs or formats for utilizing antibody structural and functional features. For example, an antibody molecule can be in a format selected from, but not limited to, intact IgA, IgG, IgE or IgM antibodies including subclasses (e.g., IgG1, IgG2, IgG3, and IgG4); multispecific (e.g., bispecific) antibodies; and/or antibody fragments (preferably antigen-binding antibody fragments). An antibody molecule can be an immunoglobulin, heavy chain antibody, light chain antibody, leucine-rich repeat (LRR) based antibody, or other protein scaffold with antibody-like properties. An antibody molecule can be any immunological binding moiety known in the art including, but not limited to, a scFv, Fab, Fab′, Fab′₂, Fab₂, Fab₃, F(ab′)₂, Fd, Fd′, Fv, Feb, diabody, triabody, tetrabody, minibody, maxibody, tandab, DVD, BiTe, TandAb, single domain antibody (e.g., shark single domain antibody, such as IgNAR or fragments thereof), camelid antibody, masked antibody (e.g., Probodies®), Small Modular ImmunoPharmaceutical (SMIP™), single chain or Tandem diabody (TandAb®), VHH, Anticalin®, Nanobody®, BiTER, ankyrin repeat protein or DARPIN®, Avimer®, DART, TCR-like antibody, Adnectin®, Affilin®, Trans-body®, Affibody®, TrimerX®, MicroProtein, Fynomer®, Centyrin®, KALBITOR®, or any combination thereof.

In some embodiments, an “antibody molecule” is or comprises a polypeptide including one or more structural elements recognized by those skilled in the art as a CDR. In some embodiments, an antibody molecule is or comprises a polypeptide including at least one CDR (e.g., at least one V_{H} CDR and/or at least one V_{L} CDR) that is substantially identical to one found in a reference antibody. In some embodiments, a CDR substantially identical to a reference CDR is either identical or contains between 1-5 amino acid substitutions compared with a reference CDR. In some embodiments a CDR is substantially identical to a reference CDR by at least 85%, 86%, 87%, 88%, 89%, 90%, 91%, 92%, 93%, 94%, 95%, 96%, 97%, 98%, 99%, or 100% sequence identity with a reference CDR. In some embodiments, a CDR is substantially identical to a reference CDR in that at least one amino acid (e.g., 1-5 amino acids) is deleted, added, or substituted compared with a reference CDR, but the CDR has an amino acid sequence that is otherwise identical to a reference CDR.

Antibody heavy chain: As used herein, the term “antibody heavy chain” refers to the larger of two types of polypeptide chains present in antibody molecules in their naturally occurring conformations.

Antibody light chain: As used herein, the term “antibody light chain” refers to the smaller of two types of polypeptide chains present in antibody molecules in their naturally occurring conformations.

Antigen: As used herein, the terms “antigen” or “Ag” refer to a molecule that is capable of provoking an immune response. This immune response may involve either antibody production, activation of specific immunologically competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to, a tissue sample, a tumor sample, a cell, or a biological fluid.

Antigen-binding fragment: As used herein, the term “antigen-binding fragment” refers to a portion of an intact antibody or recombinant variants thereof that binds a target, e.g., an antigen, to which the intact antibody binds. An antigen-binding fragment refers to the antigen-binding domain, e.g., an antigenic determining variable region of an intact antibody, which is sufficient to confer recognition and specific binding of the antibody fragment to a particular target, e.g., an antigen. An antigen-binding fragment includes any naturally occurring, enzymatically obtainable, synthetic, or genetically engineered polypeptide or glycoprotein that specifically binds an antigen to form a complex. Exemplary antigen-binding fragments include, but are not limited to, Fab, Fab′, Fab′-SH, F(ab′)₂ and Fv fragments; single-chain antibody molecules, such as scFv, VHH, or camelid; single domain antibodies, such as sdAb (either V_{L} or V_{H}); linear antibodies; or multispecific antibody molecules formed from antibody fragments, such as a fragment comprising two or more Fab fragments linked by a disulfide bridge at a hinge region. An antigen-binding fragment can be incorporated into single domain antibodies, maxibodies, minibodies, nanobodies, intrabodies, diabodies, triabodies, tetrabodies, v-NAR, or bis-scFv (see, e.g., Hollinger and Hudson, Nature Biotechnology 23:1126-1136, 2005). Antigen-binding fragments can be grafted into scaffolds based on polypeptides, such as a fibronectin type III (Fn3) (see, U.S. Pat. No. 6,703,199, which describes fibronectin polypeptide minibodies). As with intact antibody molecules, antigen-binding fragments may be mono-specific or multispecific (e.g., bispecific). A multispecific antigen-binding fragment can comprise at least two different variable domains that is each capable of specifically binding to a separate antigen or to a different epitope of the same antigen. An antigen-binding fragment may be produced by any means. For example, an antigen-binding fragment can be enzymatically or chemically produced by fragmentation of an intact antibody or antibody agent. Alternatively, an antigen-binding fragment can be recombinantly produced. An antigen-binding fragment can be wholly or partially synthetically produced. An antigen-binding fragment can have a length of at least about 50, 60, 70, 80, 90, 100, 110, 120, 130, 140, 150, 160, 170, 180, 190, or 200 amino acids or more.

Antibody-Dependent Cellular Cytotoxicity: As used herein, the terms “antibody-dependent cellular cytotoxicity” or “ADCC” refer to a phenomenon in which target cells bound by an antibody molecule are killed by immune effector cells. Without wishing to be bound by theory, ADCC is typically understood to involve Fc receptor (FcR)-bearing effector cells recognizing and subsequently killing antibody-coated target cells (e.g., cells that express on their surface specific antigens to which an antibody is bound). Immune effector cells that mediate ADCC include, but are not limited to, NK cells, macrophage, neutrophils, and eosinophils.

Associated: As used herein, two events or entities are “associated” with one another if the presence, level, degree, type and/or form of one event or entity is correlated with that of the other event or entity. For example, a particular entity (e.g., antigen, polypeptide, genetic signature, metabolite, or microbe) is considered to be associated with a particular disease, disorder, or condition, if its presence, level and/or form correlates with incidence of and/or susceptibility to the disease, disorder, or condition (e.g., across a relevant population).

Binding: As used herein, the term “binding” refers to a non-covalent association between or among two or more entities. “Direct” binding involves physical contact between entities or moieties. Indirect binding involves physical interaction by way of physical contact with one or more intermediate entities. Binding between two or more entities can typically be assessed in any of a variety of contexts-including where interacting entities or moieties are studied in isolation or in a context of more complex systems (e.g., while covalently or otherwise associated with a carrier entity and/or in a biological system or cell).

Bispecific antibody molecule: As used herein, the term “bispecific antibody molecule” refers to an antibody molecule having two distinct binding specificities. Typically, a bispecific binding molecule comprises at least two antigen-binding domains, each of which specifically binds to a different antigen or epitope. A variety of different structures of bispecific antibody molecules is known in the art. In some embodiments, each antigen-binding domain comprises an antibody molecule or antigen-finding fragment thereof including at least one V_{H} region and/or at least one V_{L} region. In some embodiments, V_{H} regions and/or V_{L} regions are from a particular monoclonal antibody. In certain embodiments, a bispecific antibody molecule comprises: (i) a first antigen-binding domain that comprises or is an antibody fragment (e.g., scFv, Fab, Fab′, F(ab′)₂, Fd, Fv, nanobody, or camelid antibody); and (ii) a second antigen-binding domain that comprises or is a full length antibody molecule.

Cancer: As used herein, the terms “cancer,” “malignancy,” “neoplasm,” “tumor,” and “carcinoma,” refer to cells that exhibit relatively abnormal, uncontrolled, and/or autonomous growth, thereby exhibiting an aberrant growth phenotype characterized by a significant loss of control of cell proliferation. Cancer cells can spread locally or through bloodstream and lymphatic system to other body parts. In some embodiments, a tumor is or comprises cells that are precancerous (e.g., benign), malignant, pre-metastatic, metastatic, and/or non-metastatic. In some embodiments, cancer is or comprises a solid tumor. In some embodiments, cancer is or comprises a hematologic tumor. Examples of various cancers are described herein and include, but are not limited to, hematopoietic cancers (e.g., leukemias, lymphomas (Hodgkin's lymphoma and non-Hodgkin's lymphoma) myelomas, and myeloproliferative disorders); sarcomas; melanomas; adenomas, carcinomas of solid tissue; squamous cell carcinomas of the mouth, throat, larynx, and lung; liver cancer; genitourinary cancers (e.g., prostate, cervical, bladder, uterine, and endometrial cancer, and renal cell carcinomas); bone cancer; pancreatic cancer; skin cancer; cutaneous or intraocular melanoma; cancer of the endocrine system, thyroid gland, or parathyroid gland; head and neck cancers; breast cancer; gastro-intestinal cancers; nervous system cancers; benign lesions, such as papillomas; or any combination thereof as well as several other types including cancers described elsewhere herein.

Carrier: As used herein, the term “carrier” refers to a diluent, adjuvant, excipient, and/or vehicle with which a composition is administered. In some exemplary embodiments, carriers include sterile liquids, such as, for example, water and oils, including oils of petroleum, animal, vegetable or synthetic origin, such as, for example, peanut oil, soybean oil, mineral oil, sesame oil, and the like. In some embodiments, carriers are or include one or more solid components.

CDR: As used herein, the terms “complementarity determining region” or “CDR” refer to sequences of amino acids within antibody variable regions that confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). Exact definitional CDR boundaries and lengths are subject to different classification and numbering systems. Certain systems have been established in the art for defining CDR boundaries (e.g., Kabat, IMGT, Chothia, or a combination thereof). CDRs may therefore be referred to by Kabat, Chothia, IMGT, or any other boundary definitions known in the art. For example, in a combined Kabat and Chothia numbering scheme, CDRs correspond to amino acid residues that are part of a Kabat CDR, a Chothia CDR, or both. Despite differing boundaries, each of these systems has some degree of overlap in what constitutes “hypervariable regions” within variable sequences. CDR definitions according to these systems may differ in length and boundary areas with respect to adjacent framework regions (see, e.g., Kabat et al., in “Sequences of Proteins of Immunological Interest,” 5th Edition, U.S. Department of Health and Human Services, 1992; Chothia et al. (1987) J. Mol. Biol. 196, 901; and MacCallum et al., J. Mol. Biol. (1996) 262, 732). Those skilled in the art appreciate differences between and among these systems and are capable of understanding CDR boundaries to an extent required to understand and to practice the claims and disclosure herein.

Chimeric antibody: As used herein, the term “chimeric antibody” refers to an antibody molecule whose amino acid sequence includes V_{H} and V_{L} region sequences that are found in a first species and constant region sequences that are found in a second different species. In many embodiments, a chimeric antibody comprises murine V_{H} and V_{L} regions linked to human constant regions. In some embodiments, an antibody with human V_{H} and V_{L} regions linked to non-human constant regions (e.g., mouse constant regions) is referred to as a “reverse chimeric antibody.”

Composition: As used herein, the term “composition” refers to a discrete physical entity that comprises one or more specified components. In general, unless otherwise specified, a composition may be of any form, e.g., gas, gel, liquid, or solid.

Comprising: As used herein, a composition or method described herein as “comprising” one or more named elements or steps is open-ended, meaning that the named element(s) or step(s) are essential, but other elements or steps may be added within the scope of the composition or method. Any composition or method described as “comprising” one or more named elements or steps also describes the corresponding, more limited composition or method “consisting essentially of” the same named elements or steps, meaning that the composition or method includes the named essential elements or steps and may also include additional elements or steps that do not materially affect the basic and novel characteristic(s) of the composition or method. Any composition or method described herein as “comprising” or “consisting essentially of” one or more named elements or steps also describes the corresponding, more limited, and closed-ended composition or method “consisting of” the named elements or steps to the exclusion of any other unnamed element or step. In any composition or method disclosed herein, known or disclosed equivalents of any named essential element or step may be substituted for that element or step.

Combination therapy: As used herein, the term “combination therapy” refers to those situations in which two or more different therapeutic agents (e.g., a bispecific antibody molecule or antigen binding fragment thereof and at least one additional agent) are administered in overlapping regimens so that the subject is simultaneously exposed to both agents. When used in combination therapy, two or more different therapeutic agents may be administered simultaneously or separately. This administration in combination can include simultaneous administration of two or more therapeutic agents in the same dosage form, simultaneous administration in separate dosage forms, and separate administration. That is, two or more therapeutic agents can be formulated together in the same dosage form and administered simultaneously. Alternatively, two or more therapeutic agents can be simultaneously administered in separate formulations. In another alternative, a first therapeutic agent can be administered followed by one or more additional therapeutic agents. In a separate administration protocol, two or more therapeutic agents may be administered a few minutes apart, a few hours apart, a few days apart, or a few weeks apart. In some embodiments, two or more therapeutic agents may be administered within hours (e.g., less than about 1 hour, about 2 hours, about 3 hours, about 4 hours, or about 5 hours) apart.

Effective amount: As used herein, the term “effective amount” refers to a dose that is adequate to prevent or treat at least one sign and/or symptom of a disease, disorder, or condition (e.g., cancer) in an individual. Amounts effective for a therapeutic or prophylactic use can depend on, for example, stage and severity of a disease, disorder, or condition being treated; age, weight, and general state of health of a patient; and judgment of a prescribing physician. Size of a dose will also be determined by the active ingredient/therapeutic selected; method of administration; timing and frequency of administration; existence, nature, and extent of any adverse side effects that might accompany the administration of a particular active; and desired physiological effect. It will be appreciated by one of skill in the art that various diseases, disorders, or conditions could require prolonged treatment involving multiple administrations. For purposes of the disclosure, an amount or dose of a therapeutic agent (e.g., one or more bispecific antibody molecules or antigen-binding fragments thereof described herein) administered should be sufficient to effect a therapeutic or prophylactic response in a subject over a reasonable time frame (e.g., reduction or other lessening of severity or duration of at least one sign or symptom). For example, a dose should be sufficient to detect, treat, or prevent cancer in a period of from about 2 hours or longer, e.g., about 12 to about 24 or more hours, from time of administration. In some embodiments, a time period is even longer. Dosing will be determined by efficacy of one or more particular therapeutic agents and condition of a subject (e.g., a human) as well as body weight of a subject (e.g., a human) to be treated.

Encoding: As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA or mRNA) or a defined sequence of amino acids and biological properties resulting therefrom. Thus, a gene encodes a protein if transcription and translation of mRNA corresponding to that gene produces the protein in a cell or other biological system. Both a coding strand (a nucleotide sequence corresponding to an mRNA sequence and typically provided in sequence listings) and a non-coding strand (used as a template for transcription of a gene or cDNA) can be referred to as encoding a protein or other product of that gene or cDNA.

Epitope: As used herein, the term “epitope” refers to any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Expression: As used herein, the term “expression” of a nucleic acid sequence refers to generation of any gene product from a nucleic acid sequence (e.g., a nucleic acid sequence encoding one or more bispecific antibody molecules or antigen-binding fragments thereof described herein). In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fragment: As used herein, the term “fragment” refers to a structure that includes a discrete portion of a whole structure, but lacks one or more moieties found in a whole structure. In some embodiments, a fragment consists of such a discrete portion. In some embodiments, a fragment comprises or is a characteristic structural element or moiety found in a whole structure. In some embodiments, a fragment (e.g., an antigen-binding fragment described herein) comprises or is at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., amino acids) as found in a whole antibody molecule. In some embodiments, fragment (e.g., an antigen-binding fragment described herein) comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of monomeric units (e.g., residues) found in a whole antibody molecule. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of monomeric units (e.g., residues) found in a whole nucleotide.

Framework region: As used herein, the terms “framework region” or “FR” refers to the sequences of a variable region minus CDRs. Because a CDR sequence can be determined by different systems, likewise a framework sequence is subject to correspondingly different interpretations. Six CDRs divide framework regions on heavy and light chains into four sub-regions (FR1, FR2, FR3, and FR4) on each chain, in which CDR1 is positioned between FR1 and FR2, CDR2 between FR2 and FR3, and CDR3 between FR3 and FR4. Without specifying particular sub-regions as FR1, FR2, FR3 or FR4, a framework region, as referred by others, represents combined FRs within a variable region of a single, naturally occurring immunoglobulin chain. As used herein, a FR represents one of four sub-regions. For example, FR1 represents a first framework region closest to the amino terminal end of a variable region and 5′ with respect to CDR1. FRs represents two or more sub-regions constituting a framework region.

HHLA2: As used herein, the terms “HHLA2” or “human endogenous retrovirus-H long terminal repeat-associating protein 2” refer to a member of the B7 family. HHLA2 is also known as HERV-H LTR-associating 2, B7y, B7H7, or B7-H7. HHLA2 protein has limited expression in normal human tissues, but is widely expressed in human cancers. HHLA2 is a membrane protein with three Ig-like domains (IgV-IgC-IgV), whereas other members of the B7 family generally have only two Ig domains (IgV-IgC). HHLA2 in normal human tissues is expressed in the epithelium of kidney, gut, gallbladder, and breast as well as placental trophoblast cells. In the immune system, HHLA2 is constitutively expressed on human monocytes and macrophages. HHLA2 regulates human T cell and HK cell functions including, for example, T cell and NK cell proliferation and cytokine production. HHLA2 is expressed in higher levels in a wide range of human cancers including, but not limited to, colorectal, renal, lung, pancreas, ovary, and prostate cancer. HHLA2 is also expressed in human cancers of thyroid, melanoma, liver, bladder, colon, kidney, breast, and esophagus tissue.

The term “HHLA2” includes fragments, variants (e.g., allelic variants), and derivatives thereof. Representative human HHLA2 cDNA and human HHLA2 protein sequences are publicly available from the National Center for Biotechnology Information (NCBI). Human HHLA2 variants include variant 1 (NM_007072.3 and NP_009003.1, which represents the longest transcript and encodes the longest isoform a), variant 2 (NM_001282556.1 and NP_001269485.1, which includes an alternate promoter and differs in the 5′ UTR compared to variant 1), variant 3 (NM_001282557.1 and NP_001269486.1, which includes an alternate promoter and differs in the 5′ UTR compared to variant 1), variant 4 (NM_001282558.1 and NP_001269487.1, which encodes isoform b, includes an alternate promoter, differs in the 5′ UTR, and lacks an alternate in-frame exon in the 3′ coding region compared to variant 1, resulting a shorter isoform than isoform a), and variant 5 (NM_001282559.1 and NP_001269488.1, which encodes isoform c, includes an alternate promoter, has a distinct 5′ UTR, and translation initiation starts at an alternate start codon compared to variant 1 resulting in a distinct N-terminus and shorter polypeptide than isoform a).

Host cell: As used herein, the term “host cell” refers to a cell into which exogenous DNA (recombinant or otherwise) has been introduced. Persons of skill in the art upon reading this disclosure will understand that such terms refer not only to the particular subject cell, but also to progeny of such a cell. Because certain modifications may occur in succeeding generations due to either mutation or environmental influences, such progeny may not, in fact, be identical to a parent cell, but are still included within the scope of the term “host cell” as used herein. In some embodiments, host cells include prokaryotic and eukaryotic cells selected from any Kingdom of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., or Streptomyces spp.), mycobacteria cells, fungal cells, yeast cells (e.g., S. cerevisiae, S. pombe, P. pastoris, or P. methanolica), plant cells, insect cells (e.g., SF-9, SF-21, baculovirus-infected insect cells, or Trichoplusia ni,), non-human animal cells, human cells, or cell fusions (e.g., hybridomas or quadromas). In some embodiments, a host cell comprises or is a human, monkey, ape, hamster, rat, or mouse cell. In some embodiments, a host cell comprises one or more of: CHO (e.g., CHO K1, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, or a cell line derived from an aforementioned cell. In some embodiments, a host cell comprises one or more viral genes.

Human antibody: As used herein, the term “human antibody” refers to antibodies having variable and constant regions generated (or assembled) from human immunoglobulin sequences. Antibodies or antigen-binding fragments thereof may be considered “human” even though their amino acid sequences include residues or elements not encoded by human germline immunoglobulin sequences (e.g., sequence variations that may have been introduced by random or site-specific mutagenesis in vitro or by somatic mutation in vivo), such as in one or more CDRs, particularly CDR3.

Humanized: As used herein, the term “humanized” refers to antibody molecules or antigen-binding fragments thereof whose amino acid sequence includes V_{H} and/or V_{L} region sequences from a reference antibody raised in a non-human species (e.g., a mouse), but also includes modifications in those sequences relative to the reference antibody intended to render them more “human-like” or more similar to human germline variable sequences. In some embodiments, a humanized antibody molecule or antigen-binding fragment thereof is one that immunospecifically binds to an antigen of interest and has a FR region with substantially an amino acid sequence of a human antibody and a CDR with substantially an amino acid sequence of a non-human antibody. A humanized antibody molecules comprises substantially all of at least one, and typically two, variable domains (Fab, Fab′, F(ab′)₂, FabC, Fv) in which all or substantially all CDR regions correspond to a non-human immunoglobulin (e.g., a donor immunoglobulin) and all or substantially all framework regions correspond to a human immunoglobulin consensus sequence. In some embodiments, a humanized antibody molecule also comprises at least a portion of an immunoglobulin constant region (Fc), typically that of a human immunoglobulin constant region. In some embodiments, a humanized antibody molecule comprises both light chain as well as at least a variable domain of a heavy chain. An antibody molecule also may include a CH1, hinge, CH2, CH3, and, optionally, a CH4 region of a heavy chain constant region. In some embodiments, a humanized antibody molecule only contains a humanized VL region. In some embodiments, a humanized antibody molecule only contains a humanized VH region. In some certain embodiments, a humanized antibody molecule contains humanized V_{H} and V_{L} regions.

Immunoglobin: As used herein, the terms “immunoglobulin” or “Ig,” refer to a class of proteins that function as antibody molecules. Antibody molecules expressed by B cells may be referred to as a BCR (B cell receptor) or antigen receptor. The five members included in this class of proteins are IgA, IgG, IgM, IgD, and IgE, which further include subclasses (e.g., IgG1, IgG2, IgG3, and IgG4). IgA is the primary antibody that is present in body secretions, such as saliva, tears, breast milk, gastrointestinal secretions, and mucus secretions of respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

Identity: As used herein, the term “identity” refers to subunit sequence identity between two polymeric molecules, particularly between two amino acid molecules, such as between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions, e.g., if half of the positions (e.g., five positions in a polymer of 10 amino acids in length) in two sequences are identical, the two sequences are 50% identical; if 90% of the positions (e.g., nine positions in a polymer of 10 amino acids in length) are identical, the two amino acids sequences are 90% identical.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Immune effector cell: As used herein, the term “immune effector cell,” refers to a cell that is involved in an immune response, e.g., promotion of an immune response. Examples of immune effector cells include, but are not limited to, T cells, natural killer (NK) cells, macrophages, monocytes, dendritic cells, neutrophils, eosinophils, mast cells, platelets, large granular lymphocytes, Langerhans' cells, or B-lymphocytes.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen. In some embodiments, an immune cell response can include proliferation of an immune effector cell (e.g., a T cell or a NK cell), cytokine production by an immune effector cell (e.g., a T cell or a NK cell), and/or release of cytotoxic granules comprising perforin and/or granzymes by an immune effector cell (e.g., a T cell or a NK cell).

“Improve,” “increase”, “inhibit” or “reduce”: As used herein, the terms “improve,” “increase,” “inhibit,” “reduce,” or grammatical equivalents thereof, indicate values that are relative to a baseline or other reference measurement. In some embodiments, an appropriate reference measurement is or comprises a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement is or comprises a measurement in comparable system known or expected to respond in a particular way, in presence of the relevant agent or treatment.

Modulating: As used herein the term “modulating,” refers to mediating a detectable increase or decrease in a level of a response and/or change in nature of a response in a subject compared with a level and/or nature of a response in a subject without a treatment or an untreated subject. The term encompasses perturbing and/or affecting a native signal or response thereby mediating a beneficial therapeutic response in a subject, preferably, a human.

Monoclonal antibody: As used herein, the terms “monoclonal antibody” or “mAb” refer to an antibody obtained from a population of substantially homogeneous antibodies, such that individual antibodies in the population are substantially identical and/or bind the same epitope, except for possible variant antibodies (e.g., containing naturally occurring mutations or arising during production of a monoclonal), such variants generally being present in minor amounts. In contrast to polyclonal antibody preparations, which typically include different antibody molecules directed against different determinants (epitopes), each monoclonal antibody molecule in such a preparation is directed against a single determinant on an antigen. Thus, “monoclonal” typically indicates a population of antibodies is not a mixture of discrete antibodies.

Nucleic acid molecule: As used herein, the term “nucleic acid molecule” refers to a polymer of at least three nucleotides. In some embodiments, a nucleic acid molecule comprises DNA. In some embodiments, a nucleic acid molecule comprises RNA. In some embodiments, a nucleic acid molecule is single stranded. In some embodiments, a nucleic acid molecule is double stranded. In some embodiments, a nucleic acid molecule comprises both single and double stranded portions. In some embodiments, a nucleic acid molecule comprises a backbone that comprises one or more phosphodiester linkages. In some embodiments, a nucleic acid molecule comprises a backbone that comprises both phosphodiester and non-phosphodiester linkages. For example, a nucleic acid molecule may comprise a backbone that comprises one or more phosphorothioate or 5′-N-phosphoramidite linkages and/or one or more peptide bonds, e.g., as in a peptide nucleic acid molecule. In some embodiments, a nucleic acid molecule comprises one or more, or all, natural residues (e.g., adenine, cytosine, deoxyadenosine, deoxycytidine, deoxyguanosine, deoxythymidine, guanine, thymine, and/or uracil). In some embodiments, a nucleic acid molecule comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, or combinations thereof). In some embodiments, a non-natural residue comprises one or more modified sugars (e.g., 2′-fluororibose, ribose, 2′-deoxyribose, arabinose, hexose, or combinations thereof) as compared to those in natural residues. In some embodiments, a nucleic acid molecule has a nucleotide sequence that encodes a functional gene product, such as an RNA or polypeptide. In some embodiments, a nucleic acid molecule has a nucleotide sequence that comprises one or more introns. In some embodiments, a nucleic acid molecule may be prepared by isolation from a natural source, enzymatic synthesis (e.g., by polymerization based on a complementary template (e.g., in vivo or in vitro), reproduction in a recombinant cell or system, or chemical synthesis. In some embodiments, a nucleic acid molecule is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Pharmaceutically acceptable: As used herein, the term “pharmaceutically acceptable” refers to compounds, materials, compositions, and/or dosage forms which are, within the scope of sound medical judgment, suitable for use in contact with tissues of human beings and animals without excessive toxicity, irritation, allergic response, or other problem or complication and commensurate with a reasonable benefit/risk ratio.

Pharmaceutically acceptable carrier: As used herein, the term “pharmaceutically acceptable carrier” refers to a pharmaceutically acceptable material, composition, or vehicle, such as a liquid or solid filler, diluent, excipient, or solvent encapsulating material, involved in carrying or transporting a subject compound from one organ, or portion of the body, to another organ, or portion of the body. Each carrier must be “acceptable” in the sense of being compatible with other ingredients of a formulation and not injurious to a subject. Some exemplary pharmaceutically acceptable carriers include, but are not limited to, sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates or polyanhydrides; or combinations thereof and other non-toxic compatible substances employed in pharmaceutical formulations.

Polypeptide: As used herein, the terms “polypeptide” or “protein” refer to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of humans. A polypeptide may comprise or consist of natural amino acids, non-natural amino acids, or both. A polypeptide may comprise or consist of only natural amino acids or only non-natural amino acids. A polypeptide may comprise D-amino acids, L-amino acids, or both. A polypeptide may include one or more pendant groups or other modifications, e.g., modifying or attached to one or more amino acid side chains at the N-terminus, at the C-terminus, or both. In some embodiments, such pendant groups or modifications are chosen from acetylation, amidation, lipidation, methylation, or pegylation, including combinations thereof. A polypeptide may be cyclic and/or may comprise a cyclic portion. In some embodiments, a polypeptide is not cyclic and/or does not comprise any cyclic portion. In some embodiments, a polypeptide is linear. A polypeptide may be or comprise a stapled polypeptide. The term “polypeptide” may be appended to a name of a reference polypeptide, activity, or structure; in such instances, it is used herein to refer to polypeptides that share relevant activity or structure and thus can be considered members of a same class or family of polypeptides. For each such class, the present disclosure provides and/or those skilled in the art will be aware of exemplary polypeptides within such class whose amino acid sequences and/or functions are known. In some embodiments, such exemplary polypeptides are reference polypeptides for a polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of a class. For example, a member polypeptide may have an overall degree of sequence homology or identity with a reference polypeptide that is at least about 30-40% and is often about 50%, 60%, 70%, 80%, 90%, 91%, 92%, 93%, 94%, 95%, 96%, 97%, 98%, 99% or more and/or includes at least one region (e.g., a conserved region that may be or comprise a characteristic sequence element) that shows very high sequence identity, often greater than 90% or even 95%, 96%, 97%, 98%, or 99%. Such a conserved region usually encompasses at least 3 to 4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. A useful polypeptide may comprise or consist of a fragment of a parent polypeptide. A useful polypeptide may comprise or consist of a plurality of fragments, each of which is found in a same parent polypeptide in a different spatial arrangement relative to one another than is found in a polypeptide of interest (e.g., fragments that are directly linked in a parent may be spatially separated in a polypeptide of interest or vice versa, and/or fragments may be present in a different order in a polypeptide of interest than in a parent), such that a polypeptide of interest is a derivative of its parent polypeptide.

scFv: As used herein, the term “scFv” refers to a fusion protein comprising at least one antibody fragment comprising a variable region of a light chain and at least one antibody fragment comprising a variable region of a heavy chain, wherein light and heavy chain variable regions are contiguously linked via a short flexible polypeptide linker. A scFv is capable of being expressed as a single chain polypeptide and retains specificity of an intact antibody from which it is derived. Unless specified, a scFv may have V_{L} and V_{H} regions in either order with respect to N-terminal and C-terminal ends of a polypeptide, such that an scFv may comprise V_{L}-linker-V_{H} or may comprise V_{H}-linker-V_{L}.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments, a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition. In some embodiments, a susceptible subject is predisposed to and/or shows an increased risk (as compared to an average risk observed in a reference subject or population) of developing a disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom one or more of diagnosis or therapy has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic: As used herein, the term “therapeutic” refers to a treatment and/or prophylaxis. A therapeutic effect is obtained, for example, by suppression, remission, or eradication of a disease state.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as the desired biological endpoint, the substance to be delivered, and/or the target cell or tissue. For example, an effective amount of a bispecific antibody molecule or antigen-binding fragment thereof in a formulation to treat a disease, disorder, or condition described herein is the amount that alleviates, ameliorates, relieves, inhibits, prevents, delays onset of, reduces severity of and/or reduces incidence of one or more symptoms or features of the disease, disorder, or condition. In some embodiments, a therapeutically effective amount is administered in a single dose. In some embodiments, multiple unit doses are required to deliver a therapeutically effective amount.

TMIGD2: As used herein, the terms “TMIGD2” or “transmembrane and immunoglobulin domain containing 2 (TMIGD2)” refer to a membrane protein having an extracellular IgV-like domain, a transmembrane region, and a proline-rich cytoplasmic domain with two tyrosine signaling motifs. TMIGD2 is constitutively expressed on naïve T cells and NK cells, but not on T regulatory cells or B cells. TMIGD2 expression is slowly lost with repetitive stimulation of T cells. Consistent with this, TMIGD2 is expressed on only about half of memory T cells, and TMIGD2-negative T cells have a terminally-differentiated, senescent phenotype. TMIGD2 is also expressed in endothelial and epithelial cells and functions to reduce cell migration and promote capillary tube formation during angiogenesis.

The term “TMIGD2” is intended to include fragments, variants (e.g., allelic variants), and derivatives thereof. Representative human TMIGD2 cDNA and human TMIGD2 protein sequences are publicly available from NCBI. Human TMIGD2 isoforms include isoform 1 (NM_144615.2 and NP 653216.2), isoform 2 (NM_001169126.1 and NP 001162597.1; which uses an alternate in-frame splice site in the 3′ coding region compared to variant 1, resulting in a shorter isoform compared to isoform 1), and isoform 3 (NM_001308232.1 and NP 001295161.1, which lacks an alternate in-frame exon in the 5′ coding region compared to variant 1, resulting in a shorter isoform, compared to isoform 1). Nucleic acid and polypeptide sequences of TMIGD2 orthologs in organisms other than humans are also known including, for example, chimpanzee TMIGD2 (XM_009434393.2 and XP_009432668.2, and XM_OO1 138228.4 and XP_001138228.3) and cattle TMIGD2 (XM 005208980.3 and XP 005209037.1, XM_005208979.3 and XP 005209036.1, and XM 002688933.5 and XP_002688979.1).

Treat: As used herein, the terms “treat,” “treatment,” or “treating” refer to partial or complete alleviation, amelioration, delay of onset of, inhibition, prevention, relief, and/or reduction in incidence and/or severity of one or more symptoms or features of a disease, disorder, and/or condition described herein. In some embodiments, treatment is administered to a subject who does not exhibit signs or features of a disease, disorder, and/or condition (e.g., may be prophylactic). In some embodiments, treatment is administered to a subject who exhibits early or mild signs or features of a disease, disorder, and/or condition described herein, for example to decrease a risk of developing pathology associated with a disease, disorder, and/or condition described herein. In some embodiments, treatment is administered to a subject who exhibits established, severe, and/or late-stage signs of a disease, disorder, and/or condition described herein. In some embodiments, treating comprises administering one or more bispecific antibody molecules or antigen-binding fragments thereof described herein to a subject.

Tumor: As used herein, the term “tumor” refers to an abnormal growth of cells or tissue. A tumor may comprise cells that are precancerous (e.g., benign), malignant, pre-metastatic, metastatic, and/or non-metastatic. In some embodiments, a tumor is associated with or is a manifestation of a cancer. In some embodiments, a tumor is a disperse tumor or a liquid tumor. In some embodiments, a tumor is a solid tumor.

Throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, and from 3 to 6, as well as individual numbers within that range, for example, 1, 1.5, 2, 2.5, 3, 3.5, 4, 4.5, 5, 5.5, and 6. This applies regardless of the breadth of the range.

### DETAILED DESCRIPTION

The present disclosure, among other things, provides bispecific antibody molecules or antigen-binding fragments thereof that can: (i) target cancer cells by binding selectively to at least one TAA; and (ii) recruit and activate immune effector cells (e.g., T cells (e.g., naïve T cells and/or effector T cells) and/or NK cells) comprising or expressing transmembrane and immunoglobulin domain containing 2 (TMIGD2) in the tumor microenvironment (TME), thereby targeting cancer cells for destruction. Accordingly, the present disclosure provides several examples of such bispecific antibody molecules or antigen-binding fragments thereof that are particularly useful for treating a variety of cancers, including solid tumors or hematological tumors, as well as modulating an immune response in a subject.

### Bispecific Antibody Molecules and Antigen-Binding Fragments Thereof

The present disclosure, among other things, provides bispecific antibody molecules or antigen-binding fragments thereof. Bispecific antibody molecules or antigen-binding fragments thereof described herein can have specificity for at least two antigens. Bispecific antibody molecules or antigen-binding fragments thereof can be characterized by a first immunoglobulin variable domain sequence that has binding specificity for a first antigen and a second immunoglobulin variable domain sequence that has binding specificity for a second antigen.

Bispecific antibody molecules or antigen-binding fragments thereof described herein can comprise: (i) a first antigen-binding domain that selectively binds to transmembrane and immunoglobulin domain containing 2 (TMIGD2), and (ii) a second antigen-binding domain that selectively bind to at least one tumor-associated antigen (TAA) described herein (e.g., HHLA2). In some embodiments, a first antigen-binding domain activates and/or recruits immune effector cells (e.g., T cells (e.g., naïve and/or effector T cells) and/or NK cells) to a tumor microenvironment (TME). In some embodiments, bispecific antibody molecules or antigen-binding fragments thereof described herein comprise: (i) a first antigen-binding domain that selectively binds TMIGD2 on immune effector cells (e.g., T cells and/or NK cells), thereby recruiting and/or activing immune effector cells in a TME; and (ii) a second antigen-binding domain that selectively binds at least one TAA described herein.

A first antigen-binding domain of a bispecific antibody molecule or antigen-binding fragment thereof described herein can selectively bind to at least one epitope on TMIGD2. A second antigen-binding domain of a bispecific antibody molecule or antigen-binding fragment thereof described herein can selectively bind to at least on epitope of a TAA described herein. In some embodiments, at least one TAA comprises or is HHLA2, CD123, B7-H3, B7-H4, CD33, CD30, HER2, CD22, CD79b, Nectin-4, Trop-2, BCMA, CD19, folate receptor a, EGFR, or combinations thereof. In certain embodiments, at least one TAA comprises or is HHLA2 (also referred to as B7y, B7H7, or B7-H7). In certain embodiments, at least one TAA comprises or is CD123. In certain embodiments, at least one TAA comprises or is B7-H3. In certain embodiments, at least one TAA comprises or is B7-H4. In certain embodiments, at least one TAA comprises or is CD33. In certain embodiments, at least one TAA comprises or is CD30. In certain embodiments, at least one TAA comprises or is HER2. In certain embodiments, at least one TAA comprises or is CD22. In certain embodiments, at least one TAA comprises or is CD79b. In certain embodiments, at least one TAA comprises or is Nectin-4. In certain embodiments, at least one TAA comprises or is Trop-2. In certain embodiments, at least one TAA comprises or is BCMA. In certain embodiments, at least one TAA comprises or is CD19. In certain embodiments, at least one TAA comprises or is folate receptor a. In certain embodiments, at least one TAA comprises or is EGFR.

In some embodiments, at least one TAA comprises or is one or more of: HHLA2 (also referred to as B7y, B7H7, or B7-H7); CD20; CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeuSAc(2-8)aNeuSAc (2-3)bDGalp(1-4) bDGlcp(1-1)Cer); a TNF receptor family member; B cell maturation (BCMA); Tn antigen (Tn Ag or GalNAca-Ser/Thr); prostate-specific membrane antigen (PSMA); receptor tyrosine kinase-like orphan receptor 1 (ROR1); FMS-like tyrosine kinase 3 (FLT3); tumor-associated glycoprotein 72 (TAG72); CD38; CD44v6; carcinoembryonic antigen (CEA); epithelial cell adhesion molecule (EPCAM); B7H3 (CD276); KIT (CD117); interleukin-13 receptor subunit alpha-2 (IL-13Ra2 or CD213A2); mesothelin; interleukin 11 receptor alpha (IL-11Ra); prostate stem cell antigen (PSCA); protease serine 21 (Testisin or PRSS21); vascular endothelial growth factor receptor 2 (VEGFR2); Lewis (Y) antigen; CD24; platelet-derived growth factor receptor beta (PDGFR-beta); stage-specific embryonic antigen-4 (SSEA-4); folate receptor alpha; HER2 (also referred to as receptor tyrosine-protein kinase erbB-2 or CD340); mucin 1, cell surface associated (MUC1); epidermal growth factor receptor (EGFR); neural cell adhesion molecule (NCAM); prostase (also referred to as kallikrein 4 (KLK4) or EMSP1); prostatic acid phosphatase (PAP); elongation factor 2 mutated (ELF2M); ephrin B2; fibroblast activation protein alpha (FAP); insulin-like growth factor 1 receptor (IGF-I receptor), carbonic anhydrase IX (CAIX); LMP2; glycoprotein 100 (gp100); oncogene fusion protein consisting of breakpoint cluster region (BCR) and abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp (1-4)bDGlcp(1-1)Cer); transglutaminase 5 (TGS5); high molecular weight-melanoma-associated antigen (HMWMAA); o-acetyl-GD2 ganglioside (OAcGD2); folate receptor beta; tumor endothelial marker 1 (TEMI/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); polysialic acid; placenta-specific 1 (PLAC1); hexasaccharide portion of globoH glycoceramide (GloboH); mammary gland differentiation antigen (NY-BR-1); uroplakin 2 (UPK2); hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); olfactory receptor 51E2 (OR51E2); TCR gamma alternate reading frame protein (TARP); Wilms tumor protein (WT1); cancer/testis antigen 1 (NY-ESO-1); cancer/testis antigen 2 (LAGE-1a); melanoma-associated antigen 1 (MAGE-A1); ETS translocation-variant gene 6, located on chromosome 12p (ETV6-AML); sperm protein 17 (SPA17); an X antigen family, such as member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); rat sarcoma (Ras) mutant; human telomerase reverse transcriptase (hTERT); sarcoma translocation breakpoints; melanoma inhibitor of apoptosis (ML-IAP); ERG; N-Acetyl glucosaminyl-transferase V (NA17); paired box protein Pax-3 (PAX3); androgen receptor; cyclin B1; v-myc avian myelocytomatosis viral oncogene neuroblastoma derived homolog (MYCN); Ras homolog family member C (RhoC); tyrosinase-related protein 2 (TRP-2); cytochrome P450 1B1 (CYP1B1); CCCTC-binding factor (Zinc Finger Protein)-like (BORIS or Brother of the Regulator of Imprinted Sites), squamous cell carcinoma antigen recognized by T cells 3 (SART3); paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); receptor for advanced glycation endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); or combinations thereof. A first antigen-binding domain of a bispecific antibody molecule or antigen-binding fragment thereof described herein can comprise or be an antibody molecule or an antigen-binding fragment thereof. A second antigen-binding domain of a bispecific antibody molecule or antigen-binding fragment thereof described herein can comprise or be an antibody molecule or an antigen-binding fragment thereof. An antibody molecule or antigen-binding fragment thereof can be any immunological binding moiety including, but not limited to, a scFv, Fab, Fab′, Fab′₂, Fab₂, Fab₃, F(ab′)₂, Fd, Fd′, Fv, Feb, diabody, triabody, tetrabody, minibody, maxibody, tandab, DVD, BiTe, TandAb, single domain antibody (e.g., shark single domain antibody, such as IgNAR or fragments thereof), camelid antibody, full length antibody, masked antibody (e.g., Probodies®), Small Modular ImmunoPharmaceutical (SMIP™), single chain or Tandem diabody (TandAb®), VHH, Anticalin®, Nanobody®, BiTE®, ankyrin repeat protein or DARPIN®, Avimer®, DART, TCR-like antibody, Adnectin®, Affilin®, Trans-body®, Affibody®, TrimerX®, MicroProtein, Fynomer®, Centyrin®, KALBITOR®, or any combination thereof.

In some embodiments, a first antigen-binding domain of a bispecific antibody molecule or antigen-binding fragment thereof described herein comprises or is an antigen-binding fragment thereof. In some embodiments, a second antigen-binding domain of a bispecific antibody molecule or antigen-binding fragment thereof described herein comprises or is an antigen-binding fragment thereof. An antigen-binding fragment can comprise or be any polypeptide or peptide-containing molecule comprising at least a portion of an immunoglobulin including at least one complementarity determining region (CDR) of a V_{H} or a V_{L} derived from any antibody, e.g. an antibody described herein. Antigen-binding fragments can be obtained using conventional techniques known to those of skill in the art, and the fragments are screened for utility in the same manner as intact antibodies. Such functional antibody fragments can retain an ability to bind selectively with TMIGD2 or at least one TAA described herein.

Examples of antigen-binding fragments described herein can include, but are not limited to: (i) a Fab fragment, a monovalent fragment comprising V_{L}, V_{H}, CL, and CH1 domains; (ii) a F(ab′)₂ fragment, a bivalent fragment comprising two Fab fragments linked by a disulfide bridge at a hinge region; (iii) a Fd fragment comprising V_{H} and CH1 domains; (iv) a Fv fragment comprising V_{L} and V_{H} domains of a single arm of an antibody, (v) a diabody (dAb) fragment comprising a V_{H} domain; (vi) a camelid or camelized variable domain; (vii) a scFv, a fusion protein of V_{H} and V_{L} regions; (viii) a single domain antibody; or (viii) a heavy chain (and a light chain (e.g., a half antibody). In certain embodiments, antigen-binding fragments described herein comprise or are scFvs.

A first antigen-binding domain of a bispecific antibody molecule or antigen-binding fragment thereof described herein can comprise or be a full length antibody, e.g., comprising an immunoglobulin Fc region. A second antigen-binding domain of a bispecific antibody molecule or antigen-binding fragment thereof described herein can comprise or be a full length antibody, e.g., comprising an Fc region. In some embodiments, an Fc region has reduced effector function relative to a reference antibody or antigen-binding fragment thereof. In some embodiments, an Fc region comprises a modification to reduce glycosylation of the Fc region. In some embodiments, a modification comprises one or more of the following amino acid substitutions: N297A, L234A, L235A, C220, C226S, C229S, P238S, C226S, C229S, E3233P, L234V, L235A, L234F, L235E, P331S, or combinations thereof, numbering according to EU index. In some aspects, the modification comprises one or more amino acid substitutions in the CH2 region at positions 234, 235, 237, or combinations thereof, numbering according to EU index.

An antibody or antigen-binding fragment thereof of a bispecific antibody molecule or antigen-binding fragment thereof described herein can include a V_{H} and V_{L} domain. In some embodiments, an antibody or antigen-binding fragment thereof comprises an immunoglobulin molecule of four polypeptide chains, e.g., two heavy chains and two light chains. A heavy chain can include a V_{H} domain and a heavy chain constant domain. A heavy chain constant domain can include CH1, hinge, CH2, CH3, and optionally, a CH4 region. A light chain can include a V_{L} domain and a light chain constant domain. A light chain constant domain can include a CL domain.

A V_{H} and/or a V_{L} can be further subdivided into regions of variability, termed CDRs, interspersed with regions that are more conserved, termed framework regions (FR). Such V_{H} and/or V_{L} domains can each include three CDRs and four framework regions, arranged from amino-terminus to carboxyl-terminus in the following order: FR1, CDR1, FR2, CDR2, FR3, CDR3, FR4, one or more of which can be engineered as described herein. In general, there are three CDRs in each V_{H} region (HCDR1, HCDR2, and HCDR3) and three CDRs in each V_{L} region (LCDR1, LCDR2, and LCDR3). Framework region and CDRs can be defined using a number of well-known schemes (see, e.g., Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software, each of which is hereby incorporated by reference in its entirety).

An antibody or antigen-binding fragment thereof of a bispecific antibody molecule or antigen-binding fragment thereof described herein can be or comprise: (a) a VH comprising one, two, or three VH CDR sequences each with at least about 80%, 85%, 90%, 91%, 92%, 93%, 94%, 95%, 96%, 97%, 98%, 99%, or more identity to a VH CDR in Tables 1-5; and/or (b) a VL comprising one, two, or three VL CDR sequences each with at least about 80%, 85%, 90%, 91%, 92%, 93%, 94%, 95%, 96%, 97%, 98%, 99%, or more identity to a VL CDR in Tables 1-5. In some embodiments, an antibody molecule or antigen-binding fragment thereof described herein is or comprises: (a) a VH comprising one, two, or three VH CDR sequences each with at least about 90%, 91%, 92%, 93%, 94%, 95%, 96%, 97%, 98%, 99%, or higher identity to a VH CDR of Tables 1-5; and/or (b) a VL comprising one, two, or three VL CDR sequences each with at least about 90%, 91%, 92%, 93%, 94%, 95%, 96%, 97%, 98%, 99%, or higher identity to a VL CDR of Tables 1-5. In some embodiments, an antibody or antigen-binding fragment thereof described herein is or comprises: (a) a VH comprising one, two, or three VH CDR sequences each comprising or consisting of a VH CDR of Tables 1-5; and/or (b) a VL comprising one, two, or three VL CDR sequences each comprising or consisting of a VL CDR of Tables 1-5.

An antibody or antigen-binding fragment thereof of a bispecific antibody molecule or antigen-binding fragment thereof described herein can be or comprise: (a) a VH with at least about 80%, 85%, 90%, 91%, 92%, 93%, 94%, 95%, 96%, 97%, 98%, 99%, or more identity to a VH in Tables 1-5; and/or (a) a VL with at least about 80%, 85%, 90%, 91%, 92%, 93%, 94%, 95%, 96%, 97%, 98%, 99%, or more identity to a VL in Tables 1-5. In some embodiments, an antibody molecule or antigen-binding fragment thereof is or comprises: (a) a VH with at least about 90%, 91%, 92%, 93%, 94%, 95%, 96%, 97%, 98%, 99%, or higher identity to a VH of Tables 1-5; and/or (b) a VL with at least about 90%, 91%, 92%, 93%, 94%, 95%, 96%, 97%, 98%, 99%, or higher identity to a VL of Tables 1-5. In some embodiments, an antibody molecule or antigen-binding fragment thereof is or comprises: (a) a VH comprising or consisting of a VH of Tables 1-5; and/or (b) a VL comprising or consisting of a VL of Tables 1-5.

An antibody or antigen-binding fragment thereof of a bispecific antibody molecule or antigen-binding fragment thereof described herein can be or comprise: (a) a heavy chain with at least about 80%, 85%, 90%, 91%, 92%, 93%, 94%, 95%, 96%, 97%, 98%, 99%, or more identity to a heavy chain in Tables 1-5; and/or (a) a light chain with at least about 80%, 85%, 90%, 91%, 92%, 93%, 94%, 95%, 96%, 97%, 98%, 99%, or more identity to a light chain in Tables 1-5. In some embodiments, an antibody molecule or antigen-binding fragment thereof is or comprises: (a) a heavy chain with at least about 90%, 91%, 92%, 93%, 94%, 95%, 96%, 97%, 98%, 99%, or higher identity to a heavy chain of Tables 1-5; and/or (b) a light chain with at least about 90%, 91%, 92%, 93%, 94%, 95%, 96%, 97%, 98%, 99%, or higher identity to a light chain of Tables 1-5. In some embodiments, an antibody molecule or antigen-binding fragment thereof is or comprises: (a) a heavy chain comprising or consisting of a heavy chain of Tables 1-5; and/or (b) a light chain comprising or consisting of a light chain of Tables 1-5.

In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 212, a VH CDR2 amino acid sequence of SEQ ID NO: 213, and a VH CDR3 amino acid sequence of SEQ ID NO: 214; and (b) a VL comprising a VL CDR1 amino acid sequence of SEQ ID NO: 223, a VL CDR2 amino acid sequence of SEQ ID NO: 224, and a VL CDR3 amino acid sequence of SEQ ID NO: 225. In some embodiments, an anti-TMIGD2 antibody or antigen-binding fragment thereof of a bispecific antibody molecule or antigen-binding fragment thereof described herein can be or comprise a VH comprising: (a) a VH CDR1 amino acid sequence of SEQ ID NO: 215, a VH CDR2 amino acid sequence of SEQ ID NO: 216, and a VH CDR3 amino acid sequence of SEQ ID NO: 217; and (b) a VL comprising a VL CDR1 amino acid sequence of SEQ ID NO: 226, a VL CDR2 amino acid sequence of SEQ ID NO: 227, and a VL CDR3 amino acid sequence of SEQ ID NO: 228. In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 218, a VH CDR2 amino acid sequence of SEQ ID NO: 219, and a VH CDR3 amino acid sequence of SEQ ID NO: 220; and (b) a VL comprising a VL CDR1 amino acid sequence of SEQ ID NO: 229, a VL CDR2 amino acid sequence of SEQ ID NO: 230, and a VL CDR3 amino acid sequence of SEQ ID NO: 231.

In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 211, or an amino acid sequence at least 85%, 90%, 95%, or 99% identical or higher to SEQ ID NO: 211. In some embodiments, an anti-TMIGD2 antibody or antigen-binding fragment thereof of a bispecific antibody molecule or antigen-binding fragment thereof described herein can be or comprise a V_{L} comprising an amino acid sequence of SEQ ID NO: 222, or an amino acid sequence at least 85%, 90%, 95%, or 99% identical or higher to SEQ ID NO: 222. In some embodiments, an anti-TMIGD2 antibody or antigen-binding fragment thereof of a bispecific antibody molecule or antigen-binding fragment thereof described herein can be or comprise a V_{H} comprising an amino acid sequence of SEQ ID NO: 211 and a V_{L} comprising an amino acid sequence of SEQ ID NO: 222.

In some embodiments, an anti-TMIGD2 antibody or antigen-binding fragment thereof of a bispecific antibody molecule or antigen-binding fragment thereof described herein comprises a linker comprising an amino acid sequence of SEQ ID NO: 221. In some embodiments, an anti-TMIGD2 antibody or antigen-binding fragment thereof of a bispecific antibody molecule or antigen-binding fragment thereof described herein comprises or has an amino acid sequence of SEQ ID NO: 209. In some embodiments, a bispecific antibody molecule or antigen-binding fragment thereof described herein comprises or has an amino acid sequence of SEQ ID NO: 205.

In some embodiments, an anti-CD20 antibody or antigen-binding fragment thereof of a bispecific antibody molecule or antigen-binding fragment thereof described herein can be or comprise a heavy chain comprising an amino acid sequence of SEQ ID NO 207:, or an amino acid sequence at least 85%, 90%, 95%, or 99% identical or higher to SEQ ID NO: 207. In some embodiments, an anti-CD20 antibody or antigen-binding fragment thereof of a bispecific antibody molecule or antigen-binding fragment thereof described herein can be or comprise a light chain comprising an amino acid sequence of SEQ ID NO: 208, or an amino acid sequence at least 85%, 90%, 95%, or 99% identical or higher to SEQ ID NO: 208. In some embodiments, an anti-CD20 antibody or antigen-binding fragment thereof of a bispecific antibody molecule or antigen-binding fragment thereof described herein can be or comprise a heavy chain comprising an amino acid sequence of SEQ ID NO: 207 and a light chain comprising an amino acid sequence of SEQ ID NO: 208.

In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 241, a VH CDR2 amino acid sequence of SEQ ID NO: 242, and a VH CDR3 amino acid sequence of SEQ ID NO: 243; and (b) a VL comprising a VL CDR1 amino acid sequence of SEQ ID NO: 252, a VL CDR2 amino acid sequence of SEQ ID NO: 253, and a VL CDR3 amino acid sequence of SEQ ID NO: 254. In some embodiments, an anti-TMIGD2 antibody or antigen-binding fragment thereof of a bispecific antibody molecule or antigen-binding fragment thereof described herein can be or comprise a VH comprising: (a) a VH CDR1 amino acid sequence of SEQ ID NO: 244, a VH CDR2 amino acid sequence of SEQ ID NO: 245, and a VH CDR3 amino acid sequence of SEQ ID NO: 246; and (b) a VL comprising a VL CDR1 amino acid sequence of SEQ ID NO: 255, a VL CDR2 amino acid sequence of SEQ ID NO: 256, and a VL CDR3 amino acid sequence of SEQ ID NO: 257. In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 247, a VH CDR2 amino acid sequence of SEQ ID NO: 248, and a VH CDR3 amino acid sequence of SEQ ID NO: 249; and (b) a VL comprising a VL CDR1 amino acid sequence of SEQ ID NO: 258, a VL CDR2 amino acid sequence of SEQ ID NO: 259, and a VL CDR3 amino acid sequence of SEQ ID NO: 260.

In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 240, or an amino acid sequence at least 85%, 90%, 95%, or 99% identical or higher to SEQ ID NO: 240. In some embodiments, an anti-TMIGD2 antibody or antigen-binding fragment thereof of a bispecific antibody molecule or antigen-binding fragment thereof described herein can be or comprise a VL comprising an amino acid sequence of SEQ ID NO: 251, or an amino acid sequence at least 85%, 90%, 95%, or 99% identical or higher to SEQ ID NO: 251. In some embodiments, an anti-TMIGD2 antibody or antigen-binding fragment thereof of a bispecific antibody molecule or antigen-binding fragment thereof described herein can be or comprise a V_{H} comprising an amino acid sequence of SEQ ID NO: 240 and a V_{L} comprising an amino acid sequence of SEQ ID NO: 251.

In some embodiments, an anti-TMIGD2 antibody or antigen-binding fragment thereof of a bispecific antibody molecule or antigen-binding fragment thereof described herein comprises a linker comprising an amino acid sequence of SEQ ID NO: 250. In some embodiments, an anti-TMIGD2 antibody or antigen-binding fragment thereof of a bispecific antibody molecule or antigen-binding fragment thereof described herein comprises or has an amino acid sequence of SEQ ID NO: 238. In some embodiments, a bispecific antibody molecule or antigen-binding fragment thereof described herein comprises or has an amino acid sequence of SEQ ID NO: 234.

In some embodiments, an anti-CD20 antibody or antigen-binding fragment thereof of a bispecific antibody molecule or antigen-binding fragment thereof described herein can be or comprise a heavy chain comprising an amino acid sequence of SEQ ID NO: 236, or an amino acid sequence at least 85%, 90%, 95%, or 99% identical or higher to SEQ ID NO: 236. In some embodiments, an anti-CD20 antibody or antigen-binding fragment thereof of a bispecific antibody molecule or antigen-binding fragment thereof described herein can be or comprise a light chain comprising an amino acid sequence of SEQ ID NO: 237, or an amino acid sequence at least 85%, 90%, 95%, or 99% identical or higher to SEQ ID NO: 237. In some embodiments, an anti-CD20 antibody or antigen-binding fragment thereof of a bispecific antibody molecule or antigen-binding fragment thereof described herein can be or comprise a heavy chain comprising an amino acid sequence of SEQ ID NO: 236 and a light chain comprising an amino acid sequence of SEQ ID NO: 237.

In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 270, a VH CDR2 amino acid sequence of SEQ ID NO: 271, and a VH CDR3 amino acid sequence of SEQ ID NO: 272; and (b) a VL comprising a VL CDR1 amino acid sequence of SEQ ID NO: 281, a VL CDR2 amino acid sequence of SEQ ID NO: 282, and a VL CDR3 amino acid sequence of SEQ ID NO: 283. In some embodiments, an anti-TMIGD2 antibody or antigen-binding fragment thereof of a bispecific antibody molecule or antigen-binding fragment thereof described herein can be or comprise a VH comprising: (a) a VH CDR1 amino acid sequence of SEQ ID NO: 273, a VH CDR2 amino acid sequence of SEQ ID NO: 274, and a VH CDR3 amino acid sequence of SEQ ID NO: 275; and (b) a VL comprising a VL CDR1 amino acid sequence of SEQ ID NO: 284, a VL CDR2 amino acid sequence of SEQ ID NO: 285, and a VL CDR3 amino acid sequence of SEQ ID NO: 286. In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 276, a VH CDR2 amino acid sequence of SEQ ID NO: 277, and a VH CDR3 amino acid sequence of SEQ ID NO: 278; and (b) a VL comprising a VL CDR1 amino acid sequence of SEQ ID NO: 287, a VL CDR2 amino acid sequence of SEQ ID NO: 288, and a VL CDR3 amino acid sequence of SEQ ID NO: 289.

In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 269, or an amino acid sequence at least 85%, 90%, 95%, or 99% identical or higher to SEQ ID NO: 269. In some embodiments, an anti-TMIGD2 antibody or antigen-binding fragment thereof of a bispecific antibody molecule or antigen-binding fragment thereof described herein can be or comprise a VL comprising an amino acid sequence of SEQ ID NO: 280, or an amino acid sequence at least 85%, 90%, 95%, or 99% identical or higher to SEQ ID NO: 280. In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 269 and a VL comprising an amino acid sequence of SEQ ID NO: 280.

In some embodiments, an anti-TMIGD2 antibody or antigen-binding fragment thereof of a bispecific antibody molecule or antigen-binding fragment thereof described herein comprises a linker comprising an amino acid sequence of SEQ ID NO: 279. In some embodiments, an anti-TMIGD2 antibody or antigen-binding fragment thereof of a bispecific antibody molecule or antigen-binding fragment thereof described herein comprises or has an amino acid sequence of SEQ ID NO: 267. In some embodiments, a bispecific antibody molecule or antigen-binding fragment thereof described herein comprises or has an amino acid sequence of SEQ ID NO: 263.

In some embodiments, an anti-CD20 antibody or antigen-binding fragment thereof of a bispecific antibody molecule or antigen-binding fragment thereof described herein can be or comprise a heavy chain comprising an amino acid sequence of SEQ ID NO: 265, or an amino acid sequence at least 85%, 90%, 95%, or 99% identical or higher to SEQ ID NO: 265. In some embodiments, an anti-CD20 antibody or antigen-binding fragment thereof of a bispecific antibody molecule or antigen-binding fragment thereof described herein can be or comprise a light chain comprising an amino acid sequence of SEQ ID NO: 266, or an amino acid sequence at least 85%, 90%, 95%, or 99% identical or higher to SEQ ID NO: 266. In some embodiments, an anti-CD20 antibody or antigen-binding fragment thereof of a bispecific antibody molecule or antigen-binding fragment thereof described herein can be or comprise a heavy chain comprising an amino acid sequence of SEQ ID NO: 265 and a light chain comprising an amino acid sequence of SEQ ID NO: 266.

In some embodiments, an anti-HHLA2 antibody or antigen-binding fragment thereof of a bispecific antibody molecule or antigen-binding fragment thereof described herein can be or comprise: (a) a VH comprising a VH CDR1 amino acid sequence of SEQ ID NO: 3, a VH CDR2 amino acid sequence of SEQ ID NO: 4, and a VH CDR3 amino acid sequence of SEQ ID NO: 5; and (b) a VL comprising a VL CDR1 amino acid sequence of SEQ ID NO: 39, a VL CDR2 amino acid sequence of SEQ ID NO: 40, and a VL CDR3 amino acid sequence of SEQ ID NO: 41. In some embodiments, an anti-HHLA2 antibody or antigen-binding fragment thereof of a bispecific antibody molecule or antigen-binding fragment thereof described herein can be or comprise a VH comprising: (a) a VH CDR1 amino acid sequence of SEQ ID NO: 6, a VH CDR2 amino acid sequence of SEQ ID NO: 7, and a VH CDR3 amino acid sequence of SEQ ID NO: 8; and (b) a VL comprising a VL CDR1 amino acid sequence of SEQ ID NO: 42, a VL CDR2 amino acid sequence of SEQ ID NO: 43, and a VL CDR3 amino acid sequence of SEQ ID NO: 44. In some embodiments, an anti-HHLA2 antibody or antigen-binding fragment thereof of a bispecific antibody molecule or antigen-binding fragment thereof described herein can be or comprise a VH comprising a VH CDR1 amino acid sequence of SEQ ID NO: 9, a VH CDR2 amino acid sequence of SEQ ID NO: 10, and a VH CDR3 amino acid sequence of SEQ ID NO: 11; and (b) a VL comprising a VL CDR1 amino acid sequence of SEQ ID NO: 45, a VL CDR2 amino acid sequence of SEQ ID NO: 46, and a VL CDR3 amino acid sequence of SEQ ID NO: 47.

In some embodiments, an anti-HHLA2 antibody or antigen-binding fragment thereof of a bispecific antibody molecule or antigen-binding fragment thereof described herein can be or comprise a VH comprising an amino acid sequence of SEQ ID NO: 12, or an amino acid sequence at least 85%, 90%, 95%, or 99% identical or higher to SEQ ID NO: 12. In some embodiments, an anti-HHLA2 antibody or antigen-binding fragment thereof of a bispecific antibody molecule or antigen-binding fragment thereof described herein can be or comprise a VL comprising an amino acid sequence of SEQ ID NO: 48, or an amino acid sequence at least 85%, 90%, 95%, or 99% identical or higher to SEQ ID NO: 48. In some embodiments, an anti-HHLA2 antibody or antigen-binding fragment thereof of a bispecific antibody molecule or antigen-binding fragment thereof described herein can be or comprise a VH comprising an amino acid sequence of SEQ ID NO: 12 and a VL comprising an amino acid sequence of SEQ ID NO: 48.

In some embodiments, an anti-HHLA2 antibody or antigen-binding fragment thereof of a bispecific antibody molecule or antigen-binding fragment thereof described herein can be or comprise a heavy chain comprising an amino acid sequence of SEQ ID NO: 14, or an amino acid sequence at least 85%, 90%, 95%, or 99% identical or higher to SEQ ID NO: 14. In some embodiments, an anti-HHLA2 antibody or antigen-binding fragment thereof of a bispecific antibody molecule or antigen-binding fragment thereof described herein can be or comprise a light chain comprising an amino acid sequence of SEQ ID NO: 50, or an amino acid sequence at least 85%, 90%, 95%, or 99% identical or higher to SEQ ID NO: 50. In some embodiments, an anti-HHLA2 antibody or antigen-binding fragment thereof of a bispecific antibody molecule or antigen-binding fragment thereof described herein can be or comprise a heavy chain comprising an amino acid sequence of SEQ ID NO: 14 and a light chain comprising an amino acid sequence of SEQ ID NO: 50.

In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 19, a VH CDR2 amino acid sequence of SEQ ID NO: 20, and a VH CDR3 amino acid sequence of SEQ ID NO: 21; and (b) a VL comprising a VL CDR1 amino acid sequence of SEQ ID NO: 30, a VL CDR2 amino acid sequence of SEQ ID NO: 31, and a VL CDR3 amino acid sequence of SEQ ID NO: 32. In some embodiments, an anti-TMIGD2 antibody or antigen-binding fragment thereof of a bispecific antibody molecule or antigen-binding fragment thereof described herein can be or comprise a VH comprising: (a) a VH CDR1 amino acid sequence of SEQ ID NO: 22, a VH CDR2 amino acid sequence of SEQ ID NO: 23, and a VH CDR3 amino acid sequence of SEQ ID NO: 24; and (b) a VL comprising a VL CDR1 amino acid sequence of SEQ ID NO: 33, a VL CDR2 amino acid sequence of SEQ ID NO: 34, and a VL CDR3 amino acid sequence of SEQ ID NO: 35. In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 25, a VH CDR2 amino acid sequence of SEQ ID NO: 26, and a VH CDR3 amino acid sequence of SEQ ID NO: 27; and (b) a VL comprising a VL CDR1 amino acid sequence of SEQ ID NO: 36, a VL CDR2 amino acid sequence of SEQ ID NO: 37, and a VL CDR3 amino acid sequence of SEQ ID NO: 38.

In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 18, or an amino acid sequence at least 85%, 90%, 95%, or 99% identical or higher to SEQ ID NO: 18. In some embodiments, an anti-TMIGD2 antibody or antigen-binding fragment thereof of a bispecific antibody molecule or antigen-binding fragment thereof described herein can be or comprise a VL comprising an amino acid sequence of SEQ ID NO: 29, or an amino acid sequence at least 85%, 90%, 95%, or 99% identical or higher to SEQ ID NO: 29. In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 18 and a VL comprising an amino acid sequence of SEQ ID NO: 29.

In some embodiments, an anti-TMIGD2 antibody or antigen-binding fragment thereof of a bispecific antibody molecule or antigen-binding fragment thereof described herein comprises a linker comprising an amino acid sequence of SEQ ID NO: 28. In some embodiments, an anti-TMIGD2 antibody or antigen-binding fragment thereof of a bispecific antibody molecule or antigen-binding fragment thereof described herein comprises or has an amino acid sequence of SEQ ID NO: 16. In some embodiments, an anti-TMIGD2 and anti-HHLA2 bispecific antibody molecule or antigen-binding fragment thereof described herein comprises or has an amino acid sequence of SEQ ID NO: 1.

In some embodiments, an anti-HHLA2 antibody or antigen-binding fragment thereof of a bispecific antibody molecule or antigen-binding fragment thereof described herein can be or comprise: (a) a VH comprising a VH CDR1 amino acid sequence of SEQ ID NO: 54, a VH CDR2 amino acid sequence of SEQ ID NO: 55, and a VH CDR3 amino acid sequence of SEQ ID NO: 56; and (b) a VL comprising a VL CDR1 amino acid sequence of SEQ ID NO: 90, a VL CDR2 amino acid sequence of SEQ ID NO: 92, and a VL CDR3 amino acid sequence of SEQ ID NO: 93. In some embodiments, an anti-HHLA2 antibody or antigen-binding fragment thereof of a bispecific antibody molecule or antigen-binding fragment thereof described herein can be or comprise a VH comprising: (a) a VH CDR1 amino acid sequence of SEQ ID NO: 57, a VH CDR2 amino acid sequence of SEQ ID NO: 58, and a VH CDR3 amino acid sequence of SEQ ID NO: 59; and (b) a VL comprising a VL CDR1 amino acid sequence of SEQ ID NO: 94, a VL CDR2 amino acid sequence of SEQ ID NO: 95, and a VL CDR3 amino acid sequence of SEQ ID NO: 96. In some embodiments, an anti-HHLA2 antibody or antigen-binding fragment thereof of a bispecific antibody molecule or antigen-binding fragment thereof described herein can be or comprise a VH comprising a VH CDR1 amino acid sequence of SEQ ID NO: 60, a VH CDR2 amino acid sequence of SEQ ID NO: 61, and a VH CDR3 amino acid sequence of SEQ ID NO: 62; and (b) a VL comprising a VL CDR1 amino acid sequence of SEQ ID NO: 97, a VL CDR2 amino acid sequence of SEQ ID NO: 98, and a VL CDR3 amino acid sequence of SEQ ID NO: 99.

In some embodiments, an anti-HHLA2 antibody or antigen-binding fragment thereof of a bispecific antibody molecule or antigen-binding fragment thereof described herein can be or comprise a VH comprising an amino acid sequence of SEQ ID NO: 63, or an amino acid sequence at least 85%, 90%, 95%, or 99% identical or higher to SEQ ID NO: 63. In some embodiments, an anti-HHLA2 antibody or antigen-binding fragment thereof of a bispecific antibody molecule or antigen-binding fragment thereof described herein can be or comprise a VL comprising an amino acid sequence of SEQ ID NO: 100, or an amino acid sequence at least 85%, 90%, 95%, or 99% identical or higher to SEQ ID NO: 100. In some embodiments, an anti-HHLA2 antibody or antigen-binding fragment thereof of a bispecific antibody molecule or antigen-binding fragment thereof described herein can be or comprise a VH comprising an amino acid sequence of SEQ ID NO: 63 and a VL comprising an amino acid sequence of SEQ ID NO: 100.

In some embodiments, an anti-HHLA2 antibody or antigen-binding fragment thereof of a bispecific antibody molecule or antigen-binding fragment thereof described herein can be or comprise a heavy chain comprising an amino acid sequence of SEQ ID NO: 65, or an amino acid sequence at least 85%, 90%, 95%, or 99% identical or higher to SEQ ID NO: 65. In some embodiments, an anti-HHLA2 antibody or antigen-binding fragment thereof of a bispecific antibody molecule or antigen-binding fragment thereof described herein can be or comprise a light chain comprising an amino acid sequence of SEQ ID NO: 102, or an amino acid sequence at least 85%, 90%, 95%, or 99% identical or higher to SEQ ID NO: 102. In some embodiments, an anti-HHLA2 antibody or antigen-binding fragment thereof of a bispecific antibody molecule or antigen-binding fragment thereof described herein can be or comprise a heavy chain comprising an amino acid sequence of SEQ ID NO: 65 and a light chain comprising an amino acid sequence of SEQ ID NO: 102.

In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 70, a VH CDR2 amino acid sequence of SEQ ID NO: 71, and a VH CDR3 amino acid sequence of SEQ ID NO: 72; and (b) a VL comprising a VL CDR1 amino acid sequence of SEQ ID NO: 81, a VL CDR2 amino acid sequence of SEQ ID NO: 82, and a VL CDR3 amino acid sequence of SEQ ID NO: 83. In some embodiments, an anti-TMIGD2 antibody or antigen-binding fragment thereof of a bispecific antibody molecule or antigen-binding fragment thereof described herein can be or comprise a VH comprising: (a) a VH CDR1 amino acid sequence of SEQ ID NO: 73, a VH CDR2 amino acid sequence of SEQ ID NO: 74, and a VH CDR3 amino acid sequence of SEQ ID NO: 75; and (b) a VL comprising a VL CDR 1 amino acid sequence of SEQ ID NO: 84, a VL CDR2 amino acid sequence of SEQ ID NO: 85, and a VL CDR3 amino acid sequence of SEQ ID NO: 86. In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 76, a VH CDR2 amino acid sequence of SEQ ID NO: 77, and a VH CDR3 amino acid sequence of SEQ ID NO: 78; and (b) a VL comprising a VL CDR1 amino acid sequence of SEQ ID NO: 87, a VL CDR2 amino acid sequence of SEQ ID NO: 88, and a VL CDR3 amino acid sequence of SEQ ID NO: 89.

In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 69, or an amino acid sequence at least 85%, 90%, 95%, or 99% identical or higher to SEQ ID NO: 69. In some embodiments, an anti-TMIGD2 antibody or antigen-binding fragment thereof of a bispecific antibody molecule or antigen-binding fragment thereof described herein can be or comprise a VL comprising an amino acid sequence of SEQ ID NO: 80, or an amino acid sequence at least 85%, 90%, 95%, or 99% identical or higher to SEQ ID NO: 80. In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 69 and a VL comprising an amino acid sequence of SEQ ID NO: 80.

In some embodiments, an anti-TMIGD2 antibody or antigen-binding fragment thereof of a bispecific antibody molecule or antigen-binding fragment thereof described herein comprises a linker comprising an amino acid sequence of SEQ ID NO: 79. In some embodiments, an anti-TMIGD2 antibody or antigen-binding fragment thereof of a bispecific antibody molecule or antigen-binding fragment thereof described herein comprises or has an amino acid sequence of SEQ ID NO: 67. In some embodiments, an anti-TMIGD2 and anti-HHLA2 bispecific antibody molecule or antigen-binding fragment thereof described herein comprises or has an amino acid sequence of SEQ ID NO: 52.

In some embodiments, an anti-HHLA2 antibody or antigen-binding fragment thereof of a bispecific antibody molecule or antigen-binding fragment thereof described herein can be or comprise: (a) a VH comprising a VH CDR1 amino acid sequence of SEQ ID NO: 105, a VH CDR2 amino acid sequence of SEQ ID NO: 106, and a VH CDR3 amino acid sequence of SEQ ID NO: 107; and (b) a VL comprising a VL CDR1 amino acid sequence of SEQ ID NO: 141, a VL CDR2 amino acid sequence of SEQ ID NO: 142, and a VL CDR3 amino acid sequence of SEQ ID NO: 143. In some embodiments, an anti-HHLA2 antibody or antigen-binding fragment thereof of a bispecific antibody molecule or antigen-binding fragment thereof described herein can be or comprise a VH comprising: (a) a VH CDR1 amino acid sequence of SEQ ID NO: 108, a VH CDR2 amino acid sequence of SEQ ID NO: 109, and a VH CDR3 amino acid sequence of SEQ ID NO: 110; and (b) a VL comprising a VL CDR1 amino acid sequence of SEQ ID NO: 144, a VL CDR2 amino acid sequence of SEQ ID NO: 145, and a VL CDR3 amino acid sequence of SEQ ID NO: 146. In some embodiments, an anti-HHLA2 antibody or antigen-binding fragment thereof of a bispecific antibody molecule or antigen-binding fragment thereof described herein can be or comprise a VH comprising a VH CDR1 amino acid sequence of SEQ ID NO: 111, a VH CDR2 amino acid sequence of SEQ ID NO: 112, and a VH CDR3 amino acid sequence of SEQ ID NO: 113; and (b) a VL comprising a VL CDR1 amino acid sequence of SEQ ID NO: 147, a VL CDR2 amino acid sequence of SEQ ID NO: 148, and a VL CDR3 amino acid sequence of SEQ ID NO: 149.

In some embodiments, an anti-HHLA2 antibody or antigen-binding fragment thereof of a bispecific antibody molecule or antigen-binding fragment thereof described herein can be or comprise a VH comprising an amino acid sequence of SEQ ID NO: 114, or an amino acid sequence at least 85%, 90%, 95%, or 99% identical or higher to SEQ ID NO: 114. In some embodiments, an anti-HHLA2 antibody or antigen-binding fragment thereof of a bispecific antibody molecule or antigen-binding fragment thereof described herein can be or comprise a VL comprising an amino acid sequence of SEQ ID NO: 150, or an amino acid sequence at least 85%, 90%, 95%, or 99% identical or higher to SEQ ID NO: 150. In some embodiments, an anti-HHLA2 antibody or antigen-binding fragment thereof of a bispecific antibody molecule or antigen-binding fragment thereof described herein can be or comprise a VH comprising an amino acid sequence of SEQ ID NO: 114 and a VL comprising an amino acid sequence of SEQ ID NO: 150.

In some embodiments, an anti-HHLA2 antibody or antigen-binding fragment thereof of a bispecific antibody molecule or antigen-binding fragment thereof described herein can be or comprise a heavy chain comprising an amino acid sequence of SEQ ID NO: 116, or an amino acid sequence at least 85%, 90%, 95%, or 99% identical or higher to SEQ ID NO: 116. In some embodiments, an anti-HHLA2 antibody or antigen-binding fragment thereof of a bispecific antibody molecule or antigen-binding fragment thereof described herein can be or comprise a light chain comprising an amino acid sequence of SEQ ID NO: 152, or an amino acid sequence at least 85%, 90%, 95%, or 99% identical or higher to SEQ ID NO: 152. In some embodiments, an anti-HHLA2 antibody or antigen-binding fragment thereof of a bispecific antibody molecule or antigen-binding fragment thereof described herein can be or comprise a heavy chain comprising an amino acid sequence of SEQ ID NO: 116 and a light chain comprising an amino acid sequence of SEQ ID NO: 152.

In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 121, a VH CDR2 amino acid sequence of SEQ ID NO: 122, and a VH CDR3 amino acid sequence of SEQ ID NO: 123; and (b) a VL comprising a VL CDR1 amino acid sequence of SEQ ID NO: 132, a VL CDR2 amino acid sequence of SEQ ID NO: 133, and a VL CDR3 amino acid sequence of SEQ ID NO: 134. In some embodiments, an anti-TMIGD2 antibody or antigen-binding fragment thereof of a bispecific antibody molecule or antigen-binding fragment thereof described herein can be or comprise a VH comprising: (a) a VH CDR1 amino acid sequence of SEQ ID NO: 124, a VH CDR2 amino acid sequence of SEQ ID NO: 125, and a VH CDR3 amino acid sequence of SEQ ID NO: 126; and (b) a VL comprising a VL CDR1 amino acid sequence of SEQ ID NO: 135, a VL CDR2 amino acid sequence of SEQ ID NO:136, and a VL CDR3 amino acid sequence of SEQ ID NO: 137. In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 127, a VH CDR2 amino acid sequence of SEQ ID NO: 128, and a VH CDR3 amino acid sequence of SEQ ID NO: 129; and (b) a VL comprising a VL CDR1 amino acid sequence of SEQ ID NO: 138, a VL CDR2 amino acid sequence of SEQ ID NO: 139, and a VL CDR3 amino acid sequence of SEQ ID NO: 140.

In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 120, or an amino acid sequence at least 85%, 90%, 95%, or 99% identical or higher to SEQ ID NO: 120. In some embodiments, an anti-TMIGD2 antibody or antigen-binding fragment thereof of a bispecific antibody molecule or antigen-binding fragment thereof described herein can be or comprise a VL comprising an amino acid sequence of SEQ ID NO: 131, or an amino acid sequence at least 85%, 90%, 95%, or 99% identical or higher to SEQ ID NO: 131. In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 120 and a VL comprising an amino acid sequence of SEQ ID NO: 131.

In some embodiments, an anti-TMIGD2 antibody or antigen-binding fragment thereof of a bispecific antibody molecule or antigen-binding fragment thereof described herein comprises a linker comprising an amino acid sequence of SEQ ID NO: 130. In some embodiments, an anti-TMIGD2 antibody or antigen-binding fragment thereof of a bispecific antibody molecule or antigen-binding fragment thereof described herein comprises or has an amino acid sequence of SEQ ID NO: 118. In some embodiments, an anti-TMIGD2 and anti-HHLA2 bispecific antibody molecule or antigen-binding fragment thereof described herein comprises or has an amino acid sequence of SEQ ID NO: 103.

In some embodiments, an anti-HHLA2 antibody or antigen-binding fragment thereof of a bispecific antibody molecule or antigen-binding fragment thereof described herein can be or comprise: (a) a VH comprising a VH CDR1 amino acid sequence of SEQ ID NO: 156, a VH CDR2 amino acid sequence of SEQ ID NO: 157, and a VH CDR3 amino acid sequence of SEQ ID NO: 158; and (b) a VL comprising a VL CDR1 amino acid sequence of SEQ ID NO: 192, a VL CDR2 amino acid sequence of SEQ ID NO: 193, and a VL CDR3 amino acid sequence of SEQ ID NO: 194. In some embodiments, an anti-HHLA2 antibody or antigen-binding fragment thereof of a bispecific antibody molecule or antigen-binding fragment thereof described herein can be or comprise a VH comprising: (a) a VH CDR1 amino acid sequence of SEQ ID NO: 159, a VH CDR2 amino acid sequence of SEQ ID NO: 160, and a VH CDR3 amino acid sequence of SEQ ID NO: 161; and (b) a VL comprising a VL CDR1 amino acid sequence of SEQ ID NO: 195, a VL CDR2 amino acid sequence of SEQ ID NO: 196, and a VL CDR3 amino acid sequence of SEQ ID NO: 197. In some embodiments, an anti-HHLA2 antibody or antigen-binding fragment thereof of a bispecific antibody molecule or antigen-binding fragment thereof described herein can be or comprise a VH comprising a VH CDR1 amino acid sequence of SEQ ID NO: 162, a VH CDR2 amino acid sequence of SEQ ID NO: 163, and a VH CDR3 amino acid sequence of SEQ ID NO: 164; and (b) a VL comprising a VL CDR1 amino acid sequence of SEQ ID NO: 198, a VL CDR2 amino acid sequence of SEQ ID NO: 199, and a VL CDR3 amino acid sequence of SEQ ID NO: 200.

In some embodiments, an anti-HHLA2 antibody or antigen-binding fragment thereof of a bispecific antibody molecule or antigen-binding fragment thereof described herein can be or comprise a VH comprising an amino acid sequence of SEQ ID NO: 165, or an amino acid sequence at least 85%, 90%, 95%, or 99% identical or higher to SEQ ID NO: 165. In some embodiments, an anti-HHLA2 antibody or antigen-binding fragment thereof of a bispecific antibody molecule or antigen-binding fragment thereof described herein can be or comprise a VL comprising an amino acid sequence of SEQ ID NO: 201, or an amino acid sequence at least 85%, 90%, 95%, or 99% identical or higher to SEQ ID NO: 201. In some embodiments, an anti-HHLA2 antibody or antigen-binding fragment thereof of a bispecific antibody molecule or antigen-binding fragment thereof described herein can be or comprise a VH comprising an amino acid sequence of SEQ ID NO: 165 and a VL comprising an amino acid sequence of SEQ ID NO: 201.

In some embodiments, an anti-HHLA2 antibody or antigen-binding fragment thereof of a bispecific antibody molecule or antigen-binding fragment thereof described herein can be or comprise a heavy chain comprising an amino acid sequence of SEQ ID NO: 167, or an amino acid sequence at least 85%, 90%, 95%, or 99% identical or higher to SEQ ID NO: 167. In some embodiments, an anti-HHLA2 antibody or antigen-binding fragment thereof of a bispecific antibody molecule or antigen-binding fragment thereof described herein can be or comprise a light chain comprising an amino acid sequence of SEQ ID NO: 203, or an amino acid sequence at least 85%, 90%, 95%, or 99% identical or higher to SEQ ID NO: 203. In some embodiments, an anti-HHLA2 antibody or antigen-binding fragment thereof of a bispecific antibody molecule or antigen-binding fragment thereof described herein can be or comprise a heavy chain comprising an amino acid sequence of SEQ ID NO: 167 and a light chain comprising an amino acid sequence of SEQ ID NO: 203.

In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 172, a VH CDR2 amino acid sequence of SEQ ID NO: 173, and a VH CDR3 amino acid sequence of SEQ ID NO: 174; and (b) a VL comprising a VL CDR1 amino acid sequence of SEQ ID NO: 183, a VL CDR2 amino acid sequence of SEQ ID NO: 184, and a VL CDR3 amino acid sequence of SEQ ID NO: 185. In some embodiments, an anti-TMIGD2 antibody or antigen-binding fragment thereof of a bispecific antibody molecule or antigen-binding fragment thereof described herein can be or comprise a VH comprising: (a) a VH CDR1 amino acid sequence of SEQ ID NO: 175, a VH CDR2 amino acid sequence of SEQ ID NO: 176, and a VH CDR3 amino acid sequence of SEQ ID NO: 177; and (b) a VL comprising a VL CDR1 amino acid sequence of SEQ ID NO: 186, a VL CDR2 amino acid sequence of SEQ ID NO:187, and a VL CDR3 amino acid sequence of SEQ ID NO: 188. In some embodiments, an anti-TMIGD2 antibody or antigen-binding fragment thereof of a bispecific antibody molecule or antigen-binding fragment thereof described herein can be or comprise: (a) a VH comprising a VH CDR1 amino acid sequence of SEQ ID NO: 178, a VH CDR2 amino acid sequence of SEQ ID NO: 179, and a VH CDR3 amino acid sequence of SEQ ID NO: 180; and (b) a VL comprising a VL CDR1 amino acid sequence of SEQ ID NO: 189, a VL CDR2 amino acid sequence of SEQ ID NO: 190, and a VL CDR3 amino acid sequence of SEQ ID NO: 191.

In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 171, or an amino acid sequence at least 85%, 90%, 95%, or 99% identical or higher to SEQ ID NO: 171. In some embodiments, an anti-TMIGD2 antibody or antigen-binding fragment thereof of a bispecific antibody molecule or antigen-binding fragment thereof described herein can be or comprise a VL comprising an amino acid sequence of SEQ ID NO: 182, or an amino acid sequence at least 85%, 90%, 95%, or 99% identical or higher to SEQ ID NO: 182. In some embodiments, an anti-TMIGD2 antibody or antigen-binding fragment thereof of a bispecific antibody molecule or antigen-binding fragment thereof described herein can be or comprise a VH comprising an amino acid sequence of SEQ ID NO: 171 and a VL comprising an amino acid sequence of SEQ ID NO: 182.

In some embodiments, an anti-TMIGD2 antibody or antigen-binding fragment thereof of a bispecific antibody molecule or antigen-binding fragment thereof described herein comprises a linker comprising an amino acid sequence of SEQ ID NO: 181. In some embodiments, an anti-TMIGD2 antibody or antigen-binding fragment thereof of a bispecific antibody molecule or antigen-binding fragment thereof described herein comprises or has an amino acid sequence of SEQ ID NO: 169. In some embodiments, an anti-TMIGD2 and anti-HHLA2 bispecific antibody molecule or antigen-binding fragment thereof described herein comprises or has an amino acid sequence of SEQ ID NO: 154.

TABLE 1
Amino acid and nucleotide sequences of exemplary bispecific antibody
molecule and antigen-binding fragments thereof.

| | | SEQ ID |
|---|---|---|
| Clone | Sequence | NO |
| TK89CB6 | Heavy Chain: IgG domain against HHLA2 + linker + scFv against TMIGD2 | |
| | IgG domain-Linker-ScFv domain-Amino acid sequence | SEQ ID |
| | QVQLVQSGAEVKKPGASVKVSCKASGYTFASYKMHWVRQA | NO: 1 |
| | PGQRLEWMGDIPAGTGNTHYSQKFQGRVTITRDTSASTAYM | |
| | ELSSLRSEDTAVYYCARDRGGYYYDSWDDWGQGTLVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKAEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKLT | |
| | VDKSRWQQGNVFSCSVMHEALHNHYTQKSLSLSPGGGGGS | |
| | GGGGSGGGGSGGGGSQVQLQQSGPGLVKPSQTLSLTCAISGD | |
| | SVSSNSATWNWIRQSPSRGLEWLGRTYYRSKWSDDYAVSVK | |
| | SRMTINADTSKNQFSLQLNSVTPEDTAVYYCARSGLDFDYGL | |
| | DVWGKGTMVTVSSGGGGSGGGGSGGGGSGGGGSQSVLTQP | |
| | PSVSGAPGQRVTISCTGSSSNIGAGYDVHWYQQLPGTAPKLLI | |
| | YGNNNRPSGVPDRFSGSKSGTSGSLAITGLQAEDEADYYCQS | |
| | YDSTLSGSVFGGGTKLTVLG | |
| | IgG domain-Linker-ScFv domain-DNA sequence | SEQ ID |
| | caggtgcagctggtgcagagcggcgcggaagtgaaaaaaccgggcgcgagcgtgaaagtg | NO: 2 |
| | agctgcaaagcgagcggctatacctttgcgagctataaaatgcattgggtgcgccaggcg | |
| | ccgggccagcgcctggaatggatgggcgatattccggcgggcaccggcaacacccattat | |
| | agccagaaatttcagggccgcgtgaccattacccgcgataccagcgcgagcaccgcgtat | |
| | atggaactgagcagcctgcgcagcgaagataccgcggtgtattattgcgcgcgcgatcgc | |
| | ggcggctattattatgatagctgggatgattggggccagggcaccctggtgaccgtgagc | |
| | agcgcgagcaccaaaggcccgagcgtgtttccgctggcgccgagcagcaaaagcaccagc | |
| | ggcggcaccgcggcgctgggctgcctggtgaaagattattttccggaaccggtgaccgtg | |
| | agctggaacagcggcgcgctgaccagcggcgtgcatacctttccggcggtgctgcagagc | |
| | agcggcctgtatagcctgagcagcgtggtgaccgtgccgagcagcagcctgggcacccag | |
| | acctatatttgcaacgtgaaccataaaccgagcaacaccaaagtggataaaaaagcggaa | |
| | ccgaaaagctgcgataaaacccatacctgcccgccgtgcccggcgccggaagcgggggc | |
| | ggcccgagcgtgtttctgtttccgccgaaaccgaaagataccctgatgattagccgcacc | |
| | ccggaagtgacctgcgtggtggtggatgtgagccatgaagatccggaagtgaaatttaac | |
| | tggtatgtggatggcgtggaagtgcataacgcgaaaaccaaaccgcgcgaagaacagtat | |
| | aacagcacctatcgcgtggtgagcgtgctgaccgtgctgcatcaggattggctgaacggc | |
| | aaagaatataaatgcaaagtgagcaacaaagcgctgccggcgccgattgaaaaaaccatt | |
| | agcaaagcgaaaggccagccgcgcgaaccgcaggtgtataccctgccgccgagccgcgat | |
| | gaactgaccaaaaaccaggtgagcctgacctgcctggtgaaaggcttttatccgagcgat | |
| | attgcggtggaatgggaaagcaacggccagccggaaaacaactataaaaccaccccgccg | |
| | gtgctggatagcgatggcagcttttttctgtatagcaaactgaccgtggataaaagccgc | |
| | tggcagcagggcaacgtgtttagctgcagcgtgatgcatgaagcgctgcataaccattat | |
| | acccagaaaagcctgagcctgagcccgggggcggcggcggcagcggcggcggcggcag | |
| | cggcggcggcggcagcggcggcggcggcagccaggtgcagctgcagcagagcggcccgg | |
| | gcctggtgaaaccgagccagaccctgagcctgacctgcgcgattagcggcgatagcgtgagc | |
| | agcaacagcgcgacctggaactggattcgccagagcccgagccgcggcctggaatggctg | |
| | ggccgcacctattatcgcagcaaatggagcgatgattatgcggtgagcgtgaaaagccgc | |
| | atgaccattaacgcggataccagcaaaaaccagtttagcctgcagctgaacagcgtgacc | |
| | ccggaagataccgcggtgtattattgcgcgcgcagcggcctggattttgattatggcctg | |
| | gatgtgtggggcaaaggcaccatggtgaccgtgagcagcggcggcggcggcagcggcggc | |
| | ggcggcagcggcggcggcggcagcggcggcggcggcagccagagcgtgctgacccagcc | |
| | gccgagcgtgagcggcgcgccgggccagcgcgtgaccattagctgcaccggcagcagcagc | |
| | aacattggcgcgggctatgatgtgcattggtatcagcagctgccgggcaccgcgccgaaa | |
| | ctgctgatttatggcaacaacaaccgcccgagcggcgtgccggatcgctttagcggcagc | |
| | aaaagcggcaccagcggcagcctggcgattaccggcctgcaggcggaagatgaagcggat | |
| | tattattgccagagctatgatagcaccctgagcggcagcgtgtttggcggcggcaccaaa | |
| | ctgaccgtgctgggc | |
| | IgG domain against HHLA2 | |
| | Heavy Chain | |
| | HCDR1 (IMGT) | SEQ ID |
| | GYTFASYK | NO: 3 |
| | HCDR2 (IMGT) | SEQ ID |
| | IPAGTGNT | NO: 4 |
| | HCDR3 (IMGT) | SEQ ID |
| | ARDRGGYYYDSWDD | NO: 5 |
| | HCDR1 (Kabat) | SEQ ID |
| | SYKMH | NO: 6 |
| | HCDR2 (Kabat) | SEQ ID |
| | DIPAGTGNTHYSQKFQG | NO: 7 |
| | HCDR3 (Kabat) | SEQ ID |
| | DRGGYYYDSWDD | NO: 8 |
| | HCDR1 (Combined Kabat/IMGT) | SEQ ID |
| | GYTFASYKMH | NO: 9 |
| | HCDR2 (Combined Kabat/IMGT) | SEQ ID |
| | DIPAGTGNTHYSQKFQG | NO: 10 |
| | HCDR3 (Combined Kabat/IMGT) | SEQ ID |
| | DRGGYYYDSWDD | NO: 11 |
| | VH-Amino acid sequence | SEQ ID |
| | QVQLVQSGAEVKKPGASVKVSCKASGYTFASYKMHWVRQ | NO: 12 |
| | APGQRLEWMGDIPAGTGNTHYSQKFQGRVTITRDTSASTAY | |
| | MELSSLRSEDTAVYYCARDRGGYYYDSWDDWGQGTLVTVS | |
| | S | |
| | VH-DNA sequence | SEQ ID |
| | CAGGTGCAGCTGGTGCAGAGCGGCGCGGAAGTGAAAAAA | NO: 13 |
| | CCGGGCGCGAGCGTGAAAGTGAGCTGCAAAGCGAGCGGC | |
| | TATACCTTTGCGAGCTATAAAATGCATTGGGTGCGCCAGG | |
| | CGCCGGGCCAGCGCCTGGAATGGATGGGCGATATTCCGGC | |
| | GGGCACCGGCAACACCCATTATAGCCAGAAATTTCAGGGC | |
| | CGCGTGACCATTACCCGCGATACCAGCGCGAGCACCGCGT | |
| | ATATGGAACTGAGCAGCCTGCGCAGCGAAGATACCGCGG | |
| | TGTATTATTGCGCGCGCGATCGCGGCGGCTATTATTATGA | |
| | TAGCTGGGATGATTGGGGCCAGGGCACCCTGGTGACCGTG | |
| | AGCAGC | |
| | Heavy chain-Amino acid sequence | SEQ ID |
| | QVQLVQSGAEVKKPGASVKVSCKASGYTFASYKMHWVRQ | NO: 14 |
| | APGQRLEWMGDIPAGTGNTHYSQKFQGRVTITRDTSASTAY | |
| | MELSSLRSEDTAVYYCARDRGGYYYDSWDDWGQGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCDKTHTCPPCPAPEAAGGPSVFLF | |
| | PPKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSRDELTKNQVSLTC | |
| | LVKGFYPSDIAVEWESNGQPENNYKTTPPVLDSDGSFFLYSK | |
| | LTVDKSRWQQGNVFSCSVMHEALHNHYTQKSLSLSPG | |
| | Heavy chain-DNA sequence | SEQ ID |
| | CAGGTGCAGCTGGTGCAGAGCGGCGCGGAAGTGAAAAAA | NO: 15 |
| | CCGGGCGCGAGCGTGAAAGTGAGCTGCAAAGCGAGCGGC | |
| | TATACCTTTGCGAGCTATAAAATGCATTGGGTGCGCCAGG | |
| | CGCCGGGCCAGCGCCTGGAATGGATGGGCGATATTCCGGC | |
| | GGGCACCGGCAACACCCATTATAGCCAGAAATTTCAGGGC | |
| | CGCGTGACCATTACCCGCGATACCAGCGCGAGCACCGCGT | |
| | ATATGGAACTGAGCAGCCTGCGCAGCGAAGATACCGCGG | |
| | TGTATTATTGCGCGCGCGATCGCGGCGGCTATTATTATGA | |
| | TAGCTGGGATGATTGGGGCCAGGGCACCCTGGTGACCGTG | |
| | AGCAGCGCGAGCACCAAAGGCCCGAGCGTGTTTCCGCTG | |
| | GCGCCGAGCAGCAAAAGCACCAGCGGCGGCACCGCGGCG | |
| | CTGGGCTGCCTGGTGAAAGATTATTTTCCGGAACCGGTGA | |
| | CCGTGAGCTGGAACAGCGGCGCGCTGACCAGCGGCGTGC | |
| | ATACCTTTCCGGCGGTGCTGCAGAGCAGCGGCCTGTATAG | |
| | CCTGAGCAGCGTGGTGACCGTGCCGAGCAGCAGCCTGGG | |
| | CACCCAGACCTATATTTGCAACGTGAACCATAAACCGAGC | |
| | AACACCAAAGTGGATAAAAAAGCGGAACCGAAAAGCTGC | |
| | GATAAAACCCATACCTGCCCGCCGTGCCCGGCGCCGGAAG | |
| | CGGCGGGCGGCCCGAGCGTGTTTCTGTTTCCGCCGAAACC | |
| | GAAAGATACCCTGATGATTAGCCGCACCCCGGAAGTGACC | |
| | TGCGTGGTGGTGGATGTGAGCCATGAAGATCCGGAAGTG | |
| | AAATTTAACTGGTATGTGGATGGCGTGGAAGTGCATAACG | |
| | CGAAAACCAAACCGCGCGAAGAACAGTATAACAGCACCT | |
| | ATCGCGTGGTGAGCGTGCTGACCGTGCTGCATCAGGATTG | |
| | GCTGAACGGCAAAGAATATAAATGCAAAGTGAGCAACAA | |
| | AGCGCTGCCGGCGCCGATTGAAAAAACCATTAGCAAAGC | |
| | GAAAGGCCAGCCGCGCGAACCGCAGGTGTATACCCTGCC | |
| | GCCGAGCCGCGATGAACTGACCAAAAACCAGGTGAGCCT | |
| | GACCTGCCTGGTGAAAGGCTTTTATCCGAGCGATATTGCG | |
| | GTGGAATGGGAAAGCAACGGCCAGCCGGAAAACAACTAT | |
| | AAAACCACCCCGCCGGTGCTGGATAGCGATGGCAGCTTTT | |
| | TTCTGTATAGCAAACTGACCGTGGATAAAAGCCGCTGGCA | |
| | GCAGGGCAACGTGTTTAGCTGCAGCGTGATGCATGAAGCG | |
| | CTGCATAACCATTATACCCAGAAAAGCCTGAGCCTGAGCC | |
| | CGGGC | |
| | ScFv against TMIGD2 | |
| | VH-Linker-VL-Amino acid sequence | SEQ ID |
| | QVQLQQSGPGLVKPSQTLSLTCAISGDSVSSNSATWNWIRQSP | NO: 16 |
| | SRGLEWLGRTYYRSKWSDDYAVSVKSRMTINADTSKNQFSL | |
| | QLNSVTPEDTAVYYCARSGLDFDYGLDVWGKGTMVTVSSG | |
| | GGGSGGGGSGGGGSGGGGSQSVLTQPPSVSGAPGQRVTISCT | |
| | GSSSNIGAGYDVHWYQQLPGTAPKLLIYGNNNRPSGVPDRFS | |
| | GSKSGTSGSLAITGLQAEDEADYYCQSYDSTLSGSVFGGGTK | |
| | LTVLG | |
| | VH-Linker-VL-DNA sequence | SEQ ID |
| | caggtgcagctgcagcagagcggcccgggcctggtgaaaccgagccagaccctgagcctg | NO: 17 |
| | acctgcgcgattagcggcgatagcgtgagcagcaacagcgcgacctggaactggattcgc | |
| | cagagcccgagccgcggcctggaatggctgggccgcacctattatcgcagcaaatggagc | |
| | gatgattatgcggtgagcgtgaaaagccgcatgaccattaacgcggataccagcaaaaac | |
| | cagtttagcctgcagctgaacagcgtgaccccggaagataccgcggtgtattattgcgcg | |
| | cgcagcggcctggattttgattatggcctggatgtgtggggcaaaggcaccatggtgacc | |
| | gtgagcagcggcggcggcggcagcggcggcggcggcagcggcggcggcggcagcggcg | |
| | gcggcggcagccagagcgtgctgacccagccgccgagcgtgagcggcgcgccgggccagc | |
| | gcgtgaccattagctgcaccggcagcagcagcaacattggcgcgggctatgatgtgcattgg | |
| | tatcagcagctgccgggcaccgcgccgaaactgctgatttatggcaacaacaaccgcccg | |
| | agcggcgtgccggatcgctttagcggcagcaaaagcggcaccagcggcagcctggcgatt | |
| | accggcctgcaggcggaagatgaagcggattattattgccagagctatgatagcaccctg | |
| | agcggcagcgtgtttggcggcggcaccaaactgaccgtgctgggc | |
| | VH Domain | SEQ ID |
| | QVQLQQSGPGLVKPSQTLSLTCAISGDSVSSNSATWNWIRQS | NO: 18 |
| | PSRGLEWLGRTYYRSKWSDDYAVSVKSRMTINADTSKNQFS | |
| | LQLNSVTPEDTAVYYCARSGLDFDYGLDVWGKGTMVTVSS | |
| | HCDR1 (IMGT) | SEQ ID |
| | GDSVSSNSAT | NO: 19 |
| | HCDR2 (IMGT) | SEQ ID |
| | TYYRSKWSD | NO: 20 |
| | HCDR3 (IMGT) | SEQ ID |
| | ARSGLDFDYGLDV | NO: 21 |
| | HCDR1 (Kabat) | SEQ ID |
| | SNSATWN | NO: 22 |
| | HCDR2 (Kabat) | SEQ ID |
| | RTYYRSKWSDDYAVSVKS | NO: 23 |
| | HCDR3 (Kabat) | SEQ ID |
| | SGLDFDYGLDV | NO: 24 |
| | HCDR1 (Combined Kabat/IMGT) | SEQ ID |
| | GDSVSSNSATWN | NO: 25 |
| | HCDR2 (Combined Kabat/IMGT) | SEQ ID |
| | RTYYRSKWSDDYAVSVKS | NO: 26 |
| | HCDR3 (Combined Kabat/IMGT) | SEQ ID |
| | SGLDFDYGLDV | NO: 27 |
| | Linker | SEQ ID |
| | GGGGSGGGGSGGGGSGGGGS | NO: 28 |
| | VL domain | SEQ ID |
| | QSVLTQPPSVSGAPGQRVTISCTGSSSNIGAGYDVHWYQQLP | NO: 29 |
| | GTAPKLLIYGNNNRPSGVPDRFSGSKSGTSGSLAITGLQAEDE | |
| | ADYYCQSYDSTLSGSVFGGGTKLTVLG | |
| | LCDR1 (IMGT) | SEQ ID |
| | SSNIGAGYD | NO: 30 |
| | LCDR2 (IMGT) | SEQ ID |
| | GN | NO: 31 |
| | LCDR3 (IMGT) | SEQ ID |
| | QSYDSTLSGSV | NO: 32 |
| | LCDR1 (Kabat) | SEQ ID |
| | TGSSSNIGAGYDVH | NO: 33 |
| | LCDR3 (Kabat) | SEQ ID |
| | QSYDSTLSGSV | NO: 34 |
| | LCDR3 (Kabat) | SEQ ID |
| | QSYDSTLSGSV | NO: 35 |
| | LCDR1 (Combined Kabat/IMGT) | SEQ ID |
| | TGSSSNIGAGYDVH | NO: 36 |
| | LCDR2 (Combined Kabat/IMGT) | SEQ ID |
| | GNNNRPS | NO: 37 |
| | LCDR3 (Combined Kabat/IMGT) | SEQ ID |
| | QSYDSTLSGSV | NO: 38 |
| | Light chain: IgG domain against HHLA2 | |
| | LCDR1 (IMGT) | SEQ ID |
| | QSISSY | NO: 39 |
| | LCDR2 (IMGT) | SEQ ID |
| | GA | NO: 40 |
| | LCDR3 (IMGT) | SEQ ID |
| | QQVPFEPPT | NO: 41 |
| | LCDR1 (Kabat) | SEQ ID |
| | RASQSISSYLN | NO: 42 |
| | LCDR2 (Kabat) | SEQ ID |
| | GASSLQS | NO: 43 |
| | LCDR3 (Kabat) | SEQ ID |
| | QQVPFEPPT | NO: 44 |
| | LCDR1 (Combined Kabat/IMGT) | SEQ ID |
| | RASQSISSYLN | NO: 45 |
| | LCDR2 (Combined Kabat/IMGT) | SEQ ID |
| | GASSLQS | NO: 46 |
| | LCDR3 (Combined Kabat/IMGT) | SEQ ID |
| | QQVPFEPPT | NO: 47 |
| | VL-Amino acid sequence | SEQ ID |
| | DIQMTQSPSSLSASVGDRVTITCRASQSISSYLNWYQQKPGK | NO: 48 |
| | APKLLIYGASSLQSGVPSRFSGSGSGTDFTLTISSLQPEDFATY | |
| | YCQQVPFEPPTFGGGTKVEIK | |
| | VL-DNA sequence | SEQ ID |
| | GACATCCAGATGACCCAGTCTCCATCCTCCCTGTCTGCATC | NO: 49 |
| | TGTAGGAGACAGAGTCACCATCACTTGCCGGGCAAGTCAG | |
| | AGCATTAGCAGCTATTTAAATTGGTATCAGCAGAAACCAG | |
| | GGAAAGCCCCTAAGCTCCTGATCTATGGTGCATCCAGTTT | |
| | GCAAAGTGGGGTCCCATCAAGGTTCAGTGGCAGTGGATCT | |
| | GGGACAGATTTCACTCTCACCATCAGCAGTCTGCAACCTG | |
| | AAGATTTTGCAACTTACTACTGTCAGCAAGTACCGTTCGA | |
| | GCCTCCTACTTTTGGCGGAGGGACCAAGGTTGAGATCAAA | |
| | Light chain-Amino acid sequence | SEQ ID |
| | DIQMTQSPSSLSASVGDRVTITCRASQSISSYLNWYQQKPGK | NO: 50 |
| | APKLLIYGASSLQSGVPSRFSGSGSGTDFTLTISSLQPEDFATY | |
| | YCQQVPFEPPTFGGGTKVEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| | Light chain-DNA sequence | SEQ ID |
| | GACATCCAGATGACCCAGTCTCCATCCTCCCTGTCTGCATC | NO: 51 |
| | TGTAGGAGACAGAGTCACCATCACTTGCCGGGCAAGTCAG | |
| | AGCATTAGCAGCTATTTAAATTGGTATCAGCAGAAACCAG | |
| | GGAAAGCCCCTAAGCTCCTGATCTATGGTGCATCCAGTTT | |
| | GCAAAGTGGGGTCCCATCAAGGTTCAGTGGCAGTGGATCT | |
| | GGGACAGATTTCACTCTCACCATCAGCAGTCTGCAACCTG | |
| | AAGATTTTGCAACTTACTACTGTCAGCAAGTACCGTTCGA | |
| | GCCTCCTACTTTTGGCGGAGGGACCAAGGTTGAGATCAAA | |
| | CGTACGGTGGCTGCACCATCTGTCTTCATCTTCCCGCCATC | |
| | TGATGAGCAGTTGAAATCTGGAACTGCCTCTGTTGTGTGC | |
| | CTGCTGAATAACTTCTATCCCAGAGAGGCCAAAGTACAGT | |
| | GGAAGGTGGATAACGCCCTCCAATCGGGTAACTCCCAGG | |
| | AGAGTGTCACAGAGCAGGACAGCAAGGACAGCACCTACA | |
| | GCCTCAGCAGCACCCTGACGCTGAGCAAAGCAGACTACG | |
| | AGAAACACAAAGTCTACGCCTGCGAAGTCACCCATCAGG | |
| | GCCTGAGCTCGCCCGTCACAAAGAGCTTCAACAGGGGAG | |
| | AGTGT | |

TABLE 2
Amino acid and nucleotide sequences of exemplary bispecific antibody
molecule and antigen-binding fragments thereof.

| | | SEQ ID |
|---|---|---|
| Clone | Sequence | NO |
| TK89CB113 | Heavy Chain: IgG domain against HHLA2 + linker + scFv against TMIGD2 | |
| | IgG domain-Linker-ScFv domain-Amino acid sequence | SEQ ID |
| | QVQLVQSGAEVKKPGASVKVSCKASGYTFASYKMHWVRQA | NO: 52 |
| | PGQRLEWMGDIPAGTGNTHYSQKFQGRVTITRDTSASTAYM | |
| | ELSSLRSEDTAVYYCARDRGGYYYDSWDDWGQGTLVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKAEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKLT | |
| | VDKSRWQQGNVFSCSVMHEALHNHYTQKSLSLSPGGGGGS | |
| | GGGGSGGGGSGGGGSQVQLQQSGPGLVKPSQTLSLTCAISGD | |
| | SVSSNSAAWNWIRQSPSRGLEWLGRTYYRSKWYNDYAVSVK | |
| | SRITINPDTSKNQFSLQMNSVTPEDTAIYYCASDVVLESSSSR | |
| | HFDYWGKGTLVTVSSGGGGSGGGGSGGGGSGGGGSEIVLTQ | |
| | SPGTLSLSPGERATLSCRASQSVSSSYLAWYQQKPGQAPRLLI | |
| | YGASSRATGIPDRFSGSGSGTDFTLTISRLEPEDFAVYFCQHYG | |
| | GSPRPFGQGTKVEIKR | |
| | IgG domain-Linker-ScFv domain-DNA sequence | SEQ ID |
| | caggtgcagctggtgcagagcggcgcggaagtgaaaaaaccgggcgcgagcgtgaaagtg | NO: 53 |
| | agctgcaaagcgagcggctatacctttgcgagctataaaatgcattgggtgcgccaggcg | |
| | ccgggccagcgcctggaatggatgggcgatattccggcgggcaccggcaacacccattat | |
| | agccagaaatttcagggccgcgtgaccattacccgcgataccagcgcgagcaccgcgtat | |
| | atggaactgagcagcctgcgcagcgaagataccgcggtgtattattgcgcgcgcgatcgc | |
| | ggcggctattattatgatagctgggatgattggggccagggcaccctggtgaccgtgagc | |
| | agcgcgagcaccaaaggcccgagcgtgtttccgctggcgccgagcagcaaaagcaccagc | |
| | ggcggcaccgcggcgctgggctgcctggtgaaagattattttccggaaccggtgaccgtg | |
| | agctggaacagcggcgcgctgaccagcggcgtgcatacctttccggcggtgctgcagagc | |
| | agcggcctgtatagcctgagcagcgtggtgaccgtgccgagcagcagcctgggcacccag | |
| | acctatatttgcaacgtgaaccataaaccgagcaacaccaaagtggataaaaaagcggaa | |
| | ccgaaaagctgcgataaaacccatacctgcccgccgtgcccggcgccggaagcggcgggc | |
| | ggcccgagcgtgtttctgtttccgccgaaaccgaaagataccctgatgattagccgcacc | |
| | ccggaagtgacctgcgtggtggtggatgtgagccatgaagatccggaagtgaaatttaac | |
| | tggtatgtggatggcgtggaagtgcataacgcgaaaaccaaaccgcgcgaagaacagtat | |
| | aacagcacctatcgcgtggtgagcgtgctgaccgtgctgcatcaggattggctgaacggc | |
| | aaagaatataaatgcaaagtgagcaacaaagcgctgccggcgccgattgaaaaaaccatt | |
| | agcaaagcgaaaggccagccgcgcgaaccgcaggtgtataccctgccgccgagccgcgat | |
| | gaactgaccaaaaaccaggtgagcctgacctgcctggtgaaaggcttttatccgagcgat | |
| | attgcggtggaatgggaaagcaacggccagccggaaaacaactataaaaccaccccgccg | |
| | gtgctggatagcgatggcagcttttttctgtatagcaaactgaccgtggataaaagccgc | |
| | tggcagcagggcaacgtgtttagctgcagcgtgatgcatgaagcgctgcataaccattat | |
| | acccagaaaagcctgagcctgagcccgggcggcggcggcggcagcggcggcggcggcag | |
| | cggcggcggcggcagcggcggcggcggcagccaggtgcagctgcagcagagcggcccgg | |
| | gcctggtgaaaccgagccagaccctgagcctgacctgcgcgattagcggcgatagcgtgagc | |
| | agcaacagcgcggcgtggaactggattcgccagagcccgagccgcggcctggaatggctg | |
| | ggccgcacctattatcgcagcaaatggtataacgattatgcggtgagcgtgaaaagccgc | |
| | attaccattaacccggataccagcaaaaaccagtttagcctgcagatgaacagcgtgacc | |
| | ccggaagataccgcgatttattattgcgcgagcgatgtggtgctggaaagcagcagcagc | |
| | cgccattttgattattggggcaaaggcaccctggtgaccgtgagcagcggcggcggcggc | |
| | agcggcggcggcggcagcggcggcggcggcagcggcggcggcggcagcgaaattgtgct | |
| | gacccagagcccgggcaccctgagcctgagcccgggcgaacgcgcgaccctgagctgccgc | |
| | gcgagccagagcgtgagcagcagctatctggcgtggtatcagcagaaaccgggccaggcg | |
| | ccgcgcctgctgatttatggcgcgagcagccgcgcgaccggcattccggatcgctttagc | |
| | ggcagcggcagcggcaccgattttaccctgaccattagccgcctggaaccggaagatttt | |
| | gcggtgtatttttgccagcattatggcggcagcccgcgcccgtttggccagggcaccaaa | |
| | gtggaaattaaacgc | |
| | IgG domain against HHLA2 | |
| | Heavy Chain | |
| | HCDR1 (IMGT) | SEQ ID |
| | GYTFASYK | NO: 54 |
| | HCDR2 (IMGT) | SEQ ID |
| | IPAGTGNT | NO: 55 |
| | HCDR3 (IMGT) | SEQ ID |
| | ARDRGGYYYDSWDD | NO: 56 |
| | HCDR1 (Kabat) | SEQ ID |
| | SYKMH | NO: 57 |
| | HCDR2 (Kabat) | SEQ ID |
| | DIPAGTGNTHYSQKFQG | NO: 58 |
| | HCDR3 (Kabat) | SEQ ID |
| | DRGGYYYDSWDD | NO: 59 |
| | HCDR1 (Combined Kabat/IMGT) | SEQ ID |
| | GYTFASYKMH | NO: 60 |
| | HCDR2 (Combined Kabat/IMGT) | SEQ ID |
| | DIPAGTGNTHYSQKFQG | NO: 61 |
| | HCDR3 (Combined Kabat/IMGT) | SEQ ID |
| | DRGGYYYDSWDD | NO: 62 |
| | VH-Amino acid sequence | SEQ ID |
| | QVQLVQSGAEVKKPGASVKVSCKASGYTFASYKMHWVRQ | NO: 63 |
| | APGQRLEWMGDIPAGTGNTHYSQKFQGRVTITRDTSASTAY | |
| | MELSSLRSEDTAVYYCARDRGGYYYDSWDDWGQGTLVTVS | |
| | S | |
| | VH-DNA sequence | SEQ ID |
| | CAGGTGCAGCTGGTGCAGAGCGGCGCGGAAGTGAAAAAA | NO: 64 |
| | CCGGGCGCGAGCGTGAAAGTGAGCTGCAAAGCGAGCGGC | |
| | TATACCTTTGCGAGCTATAAAATGCATTGGGTGCGCCAGG | |
| | CGCCGGGCCAGCGCCTGGAATGGATGGGCGATATTCCGGC | |
| | GGGCACCGGCAACACCCATTATAGCCAGAAATTTCAGGGC | |
| | CGCGTGACCATTACCCGCGATACCAGCGCGAGCACCGCGT | |
| | ATATGGAACTGAGCAGCCTGCGCAGCGAAGATACCGCGG | |
| | TGTATTATTGCGCGCGCGATCGCGGCGGCTATTATTATGA | |
| | TAGCTGGGATGATTGGGGCCAGGGCACCCTGGTGACCGTG | |
| | AGCAGC | |
| | Heavy chain-Amino acid sequence | SEQ ID |
| | QVQLVQSGAEVKKPGASVKVSCKASGYTFASYKMHWVRQ | NO: 65 |
| | APGQRLEWMGDIPAGTGNTHYSQKFQGRVTITRDTSASTAY | |
| | MELSSLRSEDTAVYYCARDRGGYYYDSWDDWGQGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCDKTHTCPPCPAPEAAGGPSVFLF | |
| | PPKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSRDELTKNQVSLTC | |
| | LVKGFYPSDIAVEWESNGQPENNYKTTPPVLDSDGSFFLYSK | |
| | LTVDKSRWQQGNVFSCSVMHEALHNHYTQKSLSLSPG | |
| | Heavy chain-DNA sequence | SEQ ID |
| | CAGGTGCAGCTGGTGCAGAGCGGCGCGGAAGTGAAAAAA | NO: 66 |
| | CCGGGCGCGAGCGTGAAAGTGAGCTGCAAAGCGAGCGGC | |
| | TATACCTTTGCGAGCTATAAAATGCATTGGGTGCGCCAGG | |
| | CGCCGGGCCAGCGCCTGGAATGGATGGGCGATATTCCGGC | |
| | GGGCACCGGCAACACCCATTATAGCCAGAAATTTCAGGGC | |
| | CGCGTGACCATTACCCGCGATACCAGCGCGAGCACCGCGT | |
| | ATATGGAACTGAGCAGCCTGCGCAGCGAAGATACCGCGG | |
| | TGTATTATTGCGCGCGCGATCGCGGCGGCTATTATTATGA | |
| | TAGCTGGGATGATTGGGGCCAGGGCACCCTGGTGACCGTG | |
| | AGCAGCGCGAGCACCAAAGGCCCGAGCGTGTTTCCGCTG | |
| | GCGCCGAGCAGCAAAAGCACCAGCGGCGGCACCGCGGCG | |
| | CTGGGCTGCCTGGTGAAAGATTATTTTCCGGAACCGGTGA | |
| | CCGTGAGCTGGAACAGCGGCGCGCTGACCAGCGGCGTGC | |
| | ATACCTTTCCGGCGGTGCTGCAGAGCAGCGGCCTGTATAG | |
| | CCTGAGCAGCGTGGTGACCGTGCCGAGCAGCAGCCTGGG | |
| | CACCCAGACCTATATTTGCAACGTGAACCATAAACCGAGC | |
| | AACACCAAAGTGGATAAAAAAGCGGAACCGAAAAGCTGC | |
| | GATAAAACCCATACCTGCCCGCCGTGCCCGGCGCCGGAAG | |
| | CGGCGGGCGGCCCGAGCGTGTTTCTGTTTCCGCCGAAACC | |
| | GAAAGATACCCTGATGATTAGCCGCACCCCGGAAGTGACC | |
| | TGCGTGGTGGTGGATGTGAGCCATGAAGATCCGGAAGTG | |
| | AAATTTAACTGGTATGTGGATGGCGTGGAAGTGCATAACG | |
| | CGAAAACCAAACCGCGCGAAGAACAGTATAACAGCACCT | |
| | ATCGCGTGGTGAGCGTGCTGACCGTGCTGCATCAGGATTG | |
| | GCTGAACGGCAAAGAATATAAATGCAAAGTGAGCAACAA | |
| | AGCGCTGCCGGCGCCGATTGAAAAAACCATTAGCAAAGC | |
| | GAAAGGCCAGCCGCGCGAACCGCAGGTGTATACCCTGCC | |
| | GCCGAGCCGCGATGAACTGACCAAAAACCAGGTGAGCCT | |
| | GACCTGCCTGGTGAAAGGCTTTTATCCGAGCGATATTGCG | |
| | GTGGAATGGGAAAGCAACGGCCAGCCGGAAAACAACTAT | |
| | AAAACCACCCCGCCGGTGCTGGATAGCGATGGCAGCTTTT | |
| | TTCTGTATAGCAAACTGACCGTGGATAAAAGCCGCTGGCA | |
| | GCAGGGCAACGTGTTTAGCTGCAGCGTGATGCATGAAGCG | |
| | CTGCATAACCATTATACCCAGAAAAGCCTGAGCCTGAGCC | |
| | CGGGC | |
| | ScFv against TMIGD2 | |
| | VH-Linker-VL-Amino acid sequence | SEQ ID |
| | QVQLQQSGPGLVKPSQTLSLTCAISGDSVSSNSAAWNWIRQS | NO: 67 |
| | PSRGLEWLGRTYYRSKWYNDYAVSVKSRITINPDTSKNQFSL | |
| | QMNSVTPEDTAIYYCASDVVLESSSSRHFDYWGKGTLVTVSS | |
| | GGGGSGGGGSGGGGSGGGGSEIVLTQSPGTLSLSPGERATLS | |
| | CRASQSVSSSYLAWYQQKPGQAPRLLIYGASSRATGIPDRFSG | |
| | SGSGTDFTLTISRLEPEDFAVYFCQHYGGSPRPFGQGTKVEIK | |
| | R | |
| | VH-Linker-VL-DNA sequence | SEQ ID |
| | caggtgcagctgcagcagagcggcccgggcctggtgaaaccgagccagaccctgagcctg | NO: 68 |
| | acctgcgcgattagcggcgatagcgtgagcagcaacagcgcggcgtggaactggattcgc | |
| | cagagcccgagccgcggcctggaatggctgggccgcacctattatcgcagcaaatggtat | |
| | aacgattatgcggtgagcgtgaaaagccgcattaccattaacccggataccagcaaaaac | |
| | cagtttagcctgcagatgaacagcgtgaccccggaagataccgcgatttattattgcgcg | |
| | agcgatgtggtgctggaaagcagcagcagccgccattttgattattggggcaaaggcacc | |
| | ctggtgaccgtgagcagcggcggcggcggcagcggcggcggcggcagcggcggcggcgg | |
| | cagcggcggcggcggcagcgaaattgtgctgacccagagcccgggcaccctgagcctgagc | |
| | ccgggcgaacgcgcgaccctgagctgccgcgcgagccagagcgtgagcagcagctatctg | |
| | gcgtggtatcagcagaaaccgggccaggcgccgcgcctgctgatttatggcgcgagcagc | |
| | cgcgcgaccggcattccggatcgctttagcggcagcggcagcggcaccgattttaccctg | |
| | accattagccgcctggaaccggaagattttgcggtgtatttttgccagcattatggcggc | |
| | agcccgcgcccgtttggccagggcaccaaagtggaaattaaacgc | |
| | VH Domain | SEQ ID |
| | QVQLQQSGPGLVKPSQTLSLTCAISGDSVSSNSAAWNWIRQS | NO: 69 |
| | PSRGLEWLGRTYYRSKWYNDYAVSVKSRITINPDTSKNQFSL | |
| | QMNSVTPEDTAIYYCASDVVLESSSSRHFDYWGKGTLVTVS | |
| | S | |
| | HCDR1 (IMGT) | SEQ ID |
| | GDSVSSNSAA | NO: 70 |
| | HCDR2 (IMGT) | SEQ ID |
| | TYYRSKWYN | NO: 71 |
| | HCDR3 (IMGT) | SEQ ID |
| | ASDVVLESSSSRHFDY | NO: 72 |
| | HCDR1 (Kabat) | SEQ ID |
| | SNSAAWN | NO: 73 |
| | HCDR2 (Kabat) | SEQ ID |
| | RTYYRSKWYNDYAVSVKS | NO: 74 |
| | HCDR3 (Kabat) | SEQ ID |
| | DVVLESSSSRHFDY | NO: 75 |
| | HCDR1 (Combined Kabat/IMGT) | SEQ ID |
| | GDSVSSNSAAWN | NO: 76 |
| | HCDR2 (Combined Kabat/IMGT) | SEQ ID |
| | RTYYRSKWYNDYAVSVKS | NO: 77 |
| | HCDR3 (Combined Kabat/IMGT) | SEQ ID |
| | DVVLESSSSRHFDY | NO: 78 |
| | Linker | SEQ ID |
| | GGGGSGGGGSGGGGSGGGGS | NO: 79 |
| | VL domain | SEQ ID |
| | EIVLTQSPGTLSLSPGERATLSCRASQSVSSSYLAWYQQKPGQ | NO: 80 |
| | APRLLIYGASSRATGIPDRFSGSGSGTDFTLTISRLEPEDFAVYF | |
| | CQHYGGSPRPFGQGTKVEIKR | |
| | LCDR1 (IMGT) | SEQ ID |
| | QSVSSSY | NO: 81 |
| | LCDR2 (IMGT) | SEQ ID |
| | GA | NO: 82 |
| | LCDR3 (IMGT) | SEQ ID |
| | QHYGGSPRP | NO: 83 |
| | LCDR1 (Kabat) | SEQ ID |
| | RASQSVSSSYLA | NO: 84 |
| | LCDR3 (Kabat) | SEQ ID |
| | GASSRAT | NO: 85 |
| | LCDR3 (Kabat) | SEQ ID |
| | QHYGGSPRP | NO: 86 |
| | LCDR1 (Combined Kabat/IMGT) | SEQ ID |
| | RASQSVSSSYLA | NO: 87 |
| | LCDR2 (Combined Kabat/IMGT) | SEQ ID |
| | GASSRAT | NO: 88 |
| | LCDR3 (Combined Kabat/IMGT) | SEQ ID |
| | QHYGGSPRP | NO: 89 |
| | Light chain: IgG domain against HHLA2 | |
| | LCDR1 (IMGT) | SEQ ID |
| | QSISSY | NO: 90 |
| | LCDR1 (IMGT) | SEQ ID |
| | QSISSY | NO: 91 |
| | LCDR2 (IMGT) | SEQ ID |
| | GA | NO: 92 |
| | LCDR3 (IMGT) | SEQ ID |
| | QQVPFEPPT | NO: 93 |
| | LCDR1 (Kabat) | SEQ ID |
| | RASQSISSYLN | NO: 94 |
| | LCDR2 (Kabat) | SEQ ID |
| | GASSLQS | NO: 95 |
| | LCDR3 (Kabat) | SEQ ID |
| | QQVPFEPPT | NO: 96 |
| | LCDR1 (Combined Kabat/IMGT) | SEQ ID |
| | RASQSISSYLN | NO: 97 |
| | LCDR2 (Combined Kabat/IMGT) | SEQ ID |
| | GASSLQS | NO: 98 |
| | LCDR3 (Combined Kabat/IMGT) | SEQ ID |
| | QQVPFEPPT | NO: 99 |
| | VL-Amino acid sequence | SEQ ID |
| | DIQMTQSPSSLSASVGDRVTITCRASQSISSYLNWYQQKPGK | NO: 100 |
| | APKLLIYGASSLQSGVPSRFSGSGSGTDFTLTISSLQPEDFATY | |
| | YCQQVPFEPPTFGGGTKVEIK | |
| | VL-DNA sequence | SEQ ID |
| | GACATCCAGATGACCCAGTCTCCATCCTCCCTGTCTGCATC | NO: 101 |
| | TGTAGGAGACAGAGTCACCATCACTTGCCGGGCAAGTCAG | |
| | AGCATTAGCAGCTATTTAAATTGGTATCAGCAGAAACCAG | |
| | GGAAAGCCCCTAAGCTCCTGATCTATGGTGCATCCAGTTT | |
| | GCAAAGTGGGGTCCCATCAAGGTTCAGTGGCAGTGGATCT | |
| | GGGACAGATTTCACTCTCACCATCAGCAGTCTGCAACCTG | |
| | AAGATTTTGCAACTTACTACTGTCAGCAAGTACCGTTCGA | |
| | GCCTCCTACTTTTGGCGGAGGGACCAAGGTTGAGATCAAA | |
| | Light chain-Amino acid sequence | SEQ ID |
| | DIQMTQSPSSLSASVGDRVTITCRASQSISSYLNWYQQKPGK | NO: 102 |
| | APKLLIYGASSLQSGVPSRFSGSGSGTDFTLTISSLQPEDFATY | |
| | YCQQVPFEPPTFGGGTKVEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |

TABLE 3
Amino acid and nucleotide sequences of exemplary bispecific antibody
molecule and antigen-binding fragments thereof.

| | | SEQ ID |
|---|---|---|
| Clone | Sequence | NO |
| TK74CB6 | Heavy Chain: IgG domain against HHLA2 + linker + scFv against TMIGD2 | |
| | IgG domain-Linker-ScFv domain-Amino acid sequence | SEQ ID |
| | EVQLVESGGGLVQPGRSLRLSCAASGFTFDDHAMFWVRQAP | NO: 103 |
| | GKGLEWVSGISWSSNSIAYADSVKGRFTISRDNAKNSLYLQM | SEQ ID |
| | NSLRAEDTAVYYCAKGSIVAGFWLGYFDLWGRGTLVTVSSA | NO: 104 |
| | STKGPSVFPLAPSSKSTSGGTAALGCLVKDYFPEPVTVSWNS | |
| | GALTSGVHTFPAVLQSSGLYSLSSVVTVPSSSLGTQTYICNVN | |
| | HKPSNTKVDKKAEPKSCDKTHTCPPCPAPEAAGGPSVFLFPP | |
| | KPKDTLMISRTPEVTCVVVDVSHEDPEVKFNWYVDGVEVHN | |
| | AKTKPREEQYNSTYRVVSVLTVLHQDWLNGKEYKCKVSNK | |
| | ALPAPIEKTISKAKGQPREPQVYTLPPSRDELTKNQVSLTCLV | |
| | KGFYPSDIAVEWESNGQPENNYKTTPPVLDSDGSFFLYSKLTV | |
| | DKSRWQQGNVFSCSVMHEALHNHYTQKSLSLSPGGGGGSG | |
| | GGGSGGGGSGGGGSQVQLQQSGPGLVKPSQTLSLTCAISGDS | |
| | VSSNSATWNWIRQSPSRGLEWLGRTYYRSKWSDDYAVSVKS | |
| | RMTINADTSKNQFSLQLNSVTPEDTAVYYCARSGLDFDYGLD | |
| | VWGKGTMVTVSSGGGGSGGGGSGGGGSGGGSQSVLTQPP | |
| | SVSGAPGQRVTISCTGSSSNIGAGYDVHWYQQLPGTAPKLLI | |
| | YGNNNRPSGVPDRFSGSKSGTSGSLAITGLQAEDEADYYCQS | |
| | YDSTLSGSVFGGGTKLTVLG | |
| | IgG domain-Linker-ScFv domain-DNA sequence | |
| | gaagtgcagctggtggaaagcggcggcggcctggtgcagccgggccgcagcctgcgcctg | |
| | agctgcgcggcgagcggctttacctttgatgatcatgcgatgttttgggtgcgccaggcg | |
| | ccgggcaaaggcctggaatgggtgagcggcattagctggagcagcaacagcattgcgtat | |
| | gcggatagcgtgaaaggccgctttaccattagccgcgataacgcgaaaaacagcctgtat | |
| | ctgcagatgaacagcctgcgcgcggaagataccgcggtgtattattgcgcgaaaggcagc | |
| | attgtggcgggcttttggctgggctattttgatctgtggggccgcggcaccctggtgacc | |
| | gtgagcagcgcgagcaccaaaggcccgagcgtgtttccgctggcgccgagcagcaaaagc | |
| | accagcggcggcaccgcggcgctgggctgcctggtgaaagattattttccggaaccggtg | |
| | accgtgagctggaacagcggcgcgctgaccagcggcgtgcatacctttccggcggtgctg | |
| | cagagcagcggcctgtatagcctgagcagcgtggtgaccgtgccgagcagcagcctgggc | |
| | acccagacctatatttgcaacgtgaaccataaaccgagcaacaccaaagtggataaaaaa | |
| | gcggaaccgaaaagctgcgataaaacccatacctgcccgccgtgcccggcgccggaagcg | |
| | gcgggcggcccgagcgtgtttctgtttccgccgaaaccgaaagataccctgatgattagc | |
| | cgcaccccggaagtgacctgcgtggtggtggatgtgagccatgaagatccggaagtgaaa | |
| | tttaactggtatgtggatggcgtggaagtgcataacgcgaaaaccaaaccgcgcgaagaa | |
| | cagtataacagcacctatcgcgtggtgagcgtgctgaccgtgctgcatcaggattggctg | |
| | aacggcaaagaatataaatgcaaagtgagcaacaaagcgctgccggcgccgattgaaaaa | |
| | accattagcaaagcgaaaggccagccgcgcgaaccgcaggtgtataccctgccgccgagc | |
| | cgcgatgaactgaccaaaaaccaggtgagcctgacctgcctggtgaaaggcttttatccg | |
| | agcgatattgcggtggaatgggaaagcaacggccagccggaaaacaactataaaaccacc | |
| | ccgccggtgctggatagcgatggcagcttttttctgtatagcaaactgaccgtggataaa | |
| | agccgctggcagcagggcaacgtgtttagctgcagcgtgatgcatgaagcgctgcataac | |
| | cattatacccagaaaagcctgagcctgagcccgggcggcggcggcggcagcggcggcggc | |
| | ggcagcggcggcggcggcagcggcggcggcggcagccaggtgcagctgcagcagagcgg | |
| | cccgggcctggtgaaaccgagccagaccctgagcctgacctgcgcgattagcggcgatagc | |
| | gtgagcagcaacagcgcgacctggaactggattcgccagagcccgagccgcggcctggaa | |
| | tggctgggccgcacctattatcgcagcaaatggagcgatgattatgcggtgagcgtgaaa | |
| | agccgcatgaccattaacgcggataccagcaaaaaccagtttagcctgcagctgaacagc | |
| | gtgaccccggaagataccgcggtgtattattgcgcgcgcagcggcctggattttgattat | |
| | ggcctggatgtgtggggcaaaggcaccatggtgaccgtgagcagcggcggcggcggcagc | |
| | ggcggcggcggcagcggcggcggcggcagcggcggcggcggcagccagagcgtgctga | |
| | cccagccgccgagcgtgagcggcgcgccgggccagcgcgtgaccattagctgcaccggcag | |
| | cagcagcaacattggcgcgggctatgatgtgcattggtatcagcagctgccgggcaccgcg | |
| | ccgaaactgctgatttatggcaacaacaaccgcccgagcggcgtgccggatcgctttagc | |
| | ggcagcaaaagcggcaccagcggcagcctggcgattaccggcctgcaggcggaagatgaa | |
| | gcggattattattgccagagctatgatagcaccctgagcggcagcgtgtttggcggcggc | |
| | accaaactgaccgtgctgggc | |
| | IgG domain against HHLA2 | |
| | Heavy Chain | |
| | HCDR1 (IMGT) | SEQ ID |
| | GFTFDDHA | NO: 105 |
| | HCDR2 (IMGT) | SEQ ID |
| | ISWSSNSI | NO: 106 |
| | HCDR3 (IMGT) | SEQ ID |
| | AKGSIVAGFWLGYFDL | NO: 107 |
| | HCDR1 (Kabat) | SEQ ID |
| | DHAMF | NO: 108 |
| | HCDR2 (Kabat) | SEQ ID |
| | GISWSSNSIAYADSVKG | NO: 109 |
| | HCDR3 (Kabat) | SEQ ID |
| | GSIVAGFWLGYFDL | NO: 110 |
| | HCDR1 (Combined Kabat/IMGT) | SEQ ID |
| | GFTFDDHAMF | NO: 111 |
| | HCDR2 (Combined Kabat/IMGT) | SEQ ID |
| | GISWSSNSIAYADSVKG | NO: 112 |
| | HCDR3 (Combined Kabat/IMGT) | SEQ ID |
| | GSIVAGFWLGYFDL | NO: 113 |
| | VH-Amino acid sequence | SEQ ID |
| | EVQLVESGGGLVQPGRSLRLSCAASGFTFDDHAMFWVRQAP | NO: 114 |
| | GKGLEWVSGISWSSNSIAYADSVKGRFTISRDNAKNSLYLQ | |
| | MNSLRAEDTAVYYCAKGSIVAGFWLGYFDLWGRGTLVTVS | |
| | S | |
| | VH-DNA sequence | SEQ ID |
| | GAAGTGCAGCTGGTGGAAAGCGGCGGCGGCCTGGTGCAG | NO: 115 |
| | CCGGGCCGCAGCCTGCGCCTGAGCTGCGCGGCGAGCGGCT | |
| | TTACCTTTGATGATCATGCGATGTTTTGGGTGCGCCAGGC | |
| | GCCGGGCAAAGGCCTGGAATGGGTGAGCGGCATTAGCTG | |
| | GAGCAGCAACAGCATTGCGTATGCGGATAGCGTGAAAGG | |
| | CCGCTTTACCATTAGCCGCGATAACGCGAAAAACAGCCTG | |
| | TATCTGCAGATGAACAGCCTGCGCGCGGAAGATACCGCG | |
| | GTGTATTATTGCGCGAAAGGCAGCATTGTGGCGGGCTTTT | |
| | GGCTGGGCTATTTTGATCTGTGGGGCCGCGGCACCCTGGT | |
| | GACCGTGAGCAGC | |
| | Heavy chain-Amino acid sequence | SEQ ID |
| | EVQLVESGGGLVQPGRSLRLSCAASGFTFDDHAMFWVRQAP | NO: 116 |
| | GKGLEWVSGISWSSNSIAYADSVKGRFTISRDNAKNSLYLQ | |
| | MNSLRAEDTAVYYCAKGSIVAGFWLGYFDLWGRGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKAEPKSCDKTHTCPPCPAPEAAGGPSVFLF | |
| | PPKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSRDELTKNQVSLTC | |
| | LVKGFYPSDIAVEWESNGQPENNYKTTPPVLDSDGSFFLYSK | |
| | LTVDKSRWQQGNVFSCSVMHEALHNHYTQKSLSLSPG | |
| | Heavy chain-DNA sequence | SEQ ID |
| | GAAGTGCAGCTGGTGGAAAGCGGCGGCGGCCTGGTGCAG | NO: 117 |
| | CCGGGCCGCAGCCTGCGCCTGAGCTGCGCGGCGAGCGGCT | |
| | TTACCTTTGATGATCATGCGATGTTTTGGGTGCGCCAGGC | |
| | GCCGGGCAAAGGCCTGGAATGGGTGAGCGGCATTAGCTG | |
| | GAGCAGCAACAGCATTGCGTATGCGGATAGCGTGAAAGG | |
| | CCGCTTTACCATTAGCCGCGATAACGCGAAAAACAGCCTG | |
| | TATCTGCAGATGAACAGCCTGCGCGCGGAAGATACCGCG | |
| | GTGTATTATTGCGCGAAAGGCAGCATTGTGGCGGGCTTTT | |
| | GGCTGGGCTATTTTGATCTGTGGGGCCGCGGCACCCTGGT | |
| | GACCGTGAGCAGCGCGAGCACCAAAGGCCCGAGCGTGTT | |
| | TCCGCTGGCGCCGAGCAGCAAAAGCACCAGCGGCGGCAC | |
| | CGCGGCGCTGGGCTGCCTGGTGAAAGATTATTTTCCGGAA | |
| | CCGGTGACCGTGAGCTGGAACAGCGGCGCGCTGACCAGC | |
| | GGCGTGCATACCTTTCCGGCGGTGCTGCAGAGCAGCGGCC | |
| | TGTATAGCCTGAGCAGCGTGGTGACCGTGCCGAGCAGCAG | |
| | CCTGGGCACCCAGACCTATATTTGCAACGTGAACCATAAA | |
| | CCGAGCAACACCAAAGTGGATAAAAAAGCGGAACCGAAA | |
| | AGCTGCGATAAAACCCATACCTGCCCGCCGTGCCCGGCGC | |
| | CGGAAGCGGCGGGCGGCCCGAGCGTGTTTCTGTTTCCGCC | |
| | GAAACCGAAAGATACCCTGATGATTAGCCGCACCCCGGA | |
| | AGTGACCTGCGTGGTGGTGGATGTGAGCCATGAAGATCCG | |
| | GAAGTGAAATTTAACTGGTATGTGGATGGCGTGGAAGTGC | |
| | ATAACGCGAAAACCAAACCGCGCGAAGAACAGTATAACA | |
| | GCACCTATCGCGTGGTGAGCGTGCTGACCGTGCTGCATCA | |
| | GGATTGGCTGAACGGCAAAGAATATAAATGCAAAGTGAG | |
| | CAACAAAGCGCTGCCGGCGCCGATTGAAAAAACCATTAG | |
| | CAAAGCGAAAGGCCAGCCGCGCGAACCGCAGGTGTATAC | |
| | CCTGCCGCCGAGCCGCGATGAACTGACCAAAAACCAGGT | |
| | GAGCCTGACCTGCCTGGTGAAAGGCTTTTATCCGAGCGAT | |
| | ATTGCGGTGGAATGGGAAAGCAACGGCCAGCCGGAAAAC | |
| | AACTATAAAACCACCCCGCCGGTGCTGGATAGCGATGGCA | |
| | GCTTTTTTCTGTATAGCAAACTGACCGTGGATAAAAGCCG | |
| | CTGGCAGCAGGGCAACGTGTTTAGCTGCAGCGTGATGCAT | |
| | GAAGCGCTGCATAACCATTATACCCAGAAAAGCCTGAGCC | |
| | TGAGCCCGGGC | |
| | ScFv against TMIGD2 | |
| | VH-Linker-VL-Amino acid sequence | SEQ ID |
| | QVQLQQSGPGLVKPSQTLSLTCAISGDSVSSNSATWNWIRQSP | NO: 118 |
| | SRGLEWLGRTYYRSKWSDDYAVSVKSRMTINADTSKNQFSL | |
| | QLNSVTPEDTAVYYCARSGLDFDYGLDVWGKGTMVTVSSG | |
| | GGGSGGGGSGGGGSGGGGSQSVLTQPPSVSGAPGQRVTISCT | |
| | GSSSNIGAGYDVHWYQQLPGTAPKLLIYGNNNRPSGVPDRFS | |
| | GSKSGTSGSLAITGLQAEDEADYYCQSYDSTLSGSVFGGGTK | |
| | LTVLG | |
| | VH-Linker-VL-DNA sequence | SEQ ID |
| | caggtgcagctgcagcagagcggcccgggcctggtgaaaccgagccagaccctgagcctg | NO: 119 |
| | acctgcgcgattagcggcgatagcgtgagcagcaacagcgcgacctggaactggattcgc | |
| | cagagcccgagccgcggcctggaatggctgggccgcacctattatcgcagcaaatggagc | |
| | gatgattatgcggtgagcgtgaaaagccgcatgaccattaacgcggataccagcaaaaac | |
| | cagtttagcctgcagctgaacagcgtgaccccggaagataccgcggtgtattattgcgcg | |
| | cgcagcggcctggattttgattatggcctggatgtgtggggcaaaggcaccatggtgacc | |
| | gtgagcagcggcggcggcggcagcggcggcggcggcagcggcggcggcggcagcggcg | |
| | gcggcggcagccagagcgtgctgacccagccgccgagcgtgagcggcgcgccgggccagc | |
| | gcgtgaccattagctgcaccggcagcagcagcaacattggcgcgggctatgatgtgcattgg | |
| | tatcagcagctgccgggcaccgcgccgaaactgctgatttatggcaacaacaaccgcccg | |
| | agcggcgtgccggatcgctttagcggcagcaaaagcggcaccagcggcagcctggcgatt | |
| | accggcctgcaggcggaagatgaagcggattattattgccagagctatgatagcaccctg | |
| | agcggcagcgtgtttggcggcggcaccaaactgaccgtgctgggc | |
| | VH Domain | SEQ ID |
| | QVQLQQSGPGLVKPSQTLSLTCAISGDSVSSNSATWNWIRQS | NO: 120 |
| | PSRGLEWLGRTYYRSKWSDDYAVSVKSRMTINADTSKNQFS | |
| | LQLNSVTPEDTAVYYCARSGLDFDYGLDVWGKGTMVTVSS | |
| | HCDR1 (IMGT) | SEQ ID |
| | GDSVSSNSAT | NO: 121 |
| | HCDR2 (IMGT) | SEQ ID |
| | TYYRSKWSD | NO: 122 |
| | HCDR3 (IMGT) | SEQ ID |
| | ARSGLDFDYGLDV | NO: 123 |
| | HCDR1 (Kabat) | SEQ ID |
| | SNSATWN | NO: 124 |
| | HCDR2 (Kabat) | SEQ ID |
| | RTYYRSKWSDDYAVSVKS | NO: 125 |
| | HCDR3 (Kabat) | SEQ ID |
| | SGLDFDYGLDV | NO: 126 |
| | HCDR1 (Combined Kabat/IMGT) | SEQ ID |
| | GDSVSSNSATWN | NO: 127 |
| | HCDR2 (Combined Kabat/IMGT) | SEQ ID |
| | RTYYRSKWSDDYAVSVKS | NO: 128 |
| | HCDR3 (Combined Kabat/IMGT) | SEQ ID |
| | SGLDFDYGLDV | NO: 129 |
| | Linker | SEQ ID |
| | GGGGSGGGGSGGGGSGGGGS | NO: 130 |
| | VL domain | SEQ ID |
| | QSVLTQPPSVSGAPGQRVTISCTGSSSNIGAGYDVHWYQQLP | NO: 131 |
| | GTAPKLLIYGNNNRPSGVPDRFSGSKSGTSGSLAITGLQAEDE | |
| | ADYYCQSYDSTLSGSVFGGGTKLTVLG | |
| | LCDR1 (IMGT) | SEQ ID |
| | SSNIGAGYD | NO: 132 |
| | LCDR2 (IMGT) | SEQ ID |
| | GN | NO: 133 |
| | LCDR3 (IMGT) | SEQ ID |
| | QSYDSTLSGSV | NO: 134 |
| | LCDR1 (Kabat) | SEQ ID |
| | TGSSSNIGAGYDVH | NO: 135 |
| | LCDR3 (Kabat) | SEQ ID |
| | GNNNRPS | NO: 136 |
| | LCDR3 (Kabat) | SEQ ID |
| | QSYDSTLSGSV | NO: 137 |
| | LCDR1 (Combined Kabat/IMGT) | SEQ ID |
| | TGSSSNIGAGYDVH | NO: 138 |
| | LCDR2 (Combined Kabat/IMGT) | SEQ ID |
| | GNNNRPS | NO: 139 |
| | LCDR3 (Combined Kabat/IMGT) | SEQ ID |
| | QSYDSTLSGSV | NO: 140 |
| | Light chain: IgG domain against HHLA2 | |
| | LCDR1 (IMGT) | SEQ ID |
| | QSVSSY | NO: 141 |
| | LCDR2 (IMGT) | SEQ ID |
| | DA | NO: 142 |
| | LCDR3 (IMGT) | SEQ ID |
| | QQVSGNPLWT | NO: 143 |
| | LCDR1 (Kabat) | SEQ ID |
| | RASQSVSSYLA | NO: 144 |
| | LCDR2 (Kabat) | SEQ ID |
| | DASNRAT | NO: 145 |
| | LCDR3 (Kabat) | SEQ ID |
| | QQVSGNPLWT | NO: 146 |
| | LCDR1 (Combined Kabat/IMGT) | SEQ ID |
| | RASQSVSSYLA | NO: 147 |
| | LCDR2 (Combined Kabat/IMGT) | SEQ ID |
| | DASNRAT | NO: 148 |
| | LCDR3 (Combined Kabat/IMGT) | SEQ ID |
| | QQVSGNPLWT | NO: 149 |
| | VL-Amino acid sequence | SEQ ID |
| | EIVLTQSPATLSLSPGERATLSCRASQSVSSYLAWYQQKPGQ | NO: 150 |
| | APRLLIYDASNRATGIPARFSGSGSGTDFTLTISSLEPEDFAVY | |
| | YCQQVSGNPLWTFGGGTKVEIK | |
| | VL-DNA sequence | SEQ ID |
| | GAAATTGTGCTGACCCAGAGCCCGGCGACCCTGAGCCTGA | NO: 151 |
| | GCCCGGGCGAACGCGCGACCCTGAGCTGCCGCGCGAGCC | |
| | AGAGCGTGAGCAGCTATCTGGCGTGGTATCAGCAGAAAC | |
| | CGGGCCAGGCGCCGCGCCTGCTGATTTATGATGCGAGCAA | |
| | CCGCGCGACCGGCATTCCGGCGCGCTTTAGCGGCAGCGGC | |
| | AGCGGCACCGATTTTACCCTGACCATTAGCAGCCTGGAAC | |
| | CGGAAGATTTTGCGGTGTATTATTGCCAGCAGGTGAGCGG | |
| | CAACCCGCTGTGGACCTTTGGCGGCGGCACCAAAGTGGAA | |
| | ATTAAA | |
| | Light chain-Amino acid sequence | SEQ ID |
| | EIVLTQSPATLSLSPGERATLSCRASQSVSSYLAWYQQKPGQ | NO: 152 |
| | APRLLIYDASNRATGIPARFSGSGSGTDFTLTISSLEPEDFAVY | |
| | YCQQVSGNPLWTFGGGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| | Light chain-DNA sequence | SEQ ID |
| | GAAATTGTGCTGACCCAGAGCCCGGCGACCCTGAGCCTGA | NO: 153 |
| | GCCCGGGCGAACGCGCGACCCTGAGCTGCCGCGCGAGCC | |
| | AGAGCGTGAGCAGCTATCTGGCGTGGTATCAGCAGAAAC | |
| | CGGGCCAGGCGCCGCGCCTGCTGATTTATGATGCGAGCAA | |
| | CCGCGCGACCGGCATTCCGGCGCGCTTTAGCGGCAGCGGC | |
| | AGCGGCACCGATTTTACCCTGACCATTAGCAGCCTGGAAC | |
| | CGGAAGATTTTGCGGTGTATTATTGCCAGCAGGTGAGCGG | |
| | CAACCCGCTGTGGACCTTTGGCGGCGGCACCAAAGTGGAA | |
| | ATTAAACGCACCGTGGCGGCGCCGAGCGTGTTTATTTTTC | |
| | CGCCGAGCGATGAACAGCTGAAAAGCGGCACCGCGAGCG | |
| | TGGTGTGCCTGCTGAACAACTTTTATCCGCGCGAAGCGAA | |
| | AGTGCAGTGGAAAGTGGATAACGCGCTGCAGAGCGGCAA | |
| | CAGCCAGGAAAGCGTGACCGAACAGGATAGCAAAGATAG | |
| | CACCTATAGCCTGAGCAGCACCCTGACCCTGAGCAAAGCG | |
| | GATTATGAAAAACATAAAGTGTATGCGTGCGAAGTGACCC | |
| | ATCAGGGCCTGAGCAGCCCGGTGACCAAAAGCTTTAACCG | |
| | CGGCGAATGC | |

TABLE 4
Amino acid and nucleotide sequences of exemplary bispecific antibody
molecule and antigen-binding fragments thereof.

| | | SEQ ID |
|---|---|---|
| Clone | Sequence | NO |
| TK74CB113 | Heavy Chain: IgG domain against HHLA2 + linker + scFv against TMIGD2 | |
| | IgG domain-Linker-ScFv domain-Amino acid sequence | SEQ ID |
| | EVQLVESGGGLVQPGRSLRLSCAASGFTFDDHAMFWVRQAP | NO: 154 |
| | GKGLEWVSGISWSSNSIAYADSVKGRFTISRDNAKNSLYLQM | |
| | NSLRAEDTAVYYCAKGSIVAGFWLGYFDLWGRGTLVTVSSA | |
| | STKGPSVFPLAPSSKSTSGGTAALGCLVKDYFPEPVTVSWNS | |
| | GALTSGVHTFPAVLQSSGLYSLSSVVTVPSSSLGTQTYICNVN | |
| | HKPSNTKVDKKAEPKSCDKTHTCPPCPAPEAAGGPSVFLFPP | |
| | KPKDTLMISRTPEVTCVVVDVSHEDPEVKFNWYVDGVEVHN | |
| | AKTKPREEQYNSTYRVVSVLTVLHQDWLNGKEYKCKVSNK | |
| | ALPAPIEKTISKAKGQPREPQVYTLPPSRDELTKNQVSLTCLV | |
| | KGFYPSDIAVEWESNGQPENNYKTTPPVLDSDGSFFLYSKLTV | |
| | DKSRWQQGNVFSCSVMHEALHNHYTQKSLSLSPGGGGGSG | |
| | GGGSGGGGSGGGGSQVQLQQSGPGLVKPSQTLSLTCAISGDS | |
| | VSSNSAAWNWIRQSPSRGLEWLGRTYYRSKWYNDYAVSVKS | |
| | RITINPDTSKNQFSLQMNSVTPEDTAIYYCASDVVLESSSSRH | |
| | FDYWGKGTLVTVSSGGGGSGGGGSGGGGSGGGGSEIVLTQS | |
| | PGTLSLSPGERATLSCRASQSVSSSYLAWYQQKPGQAPRLLIY | |
| | GASSRATGIPDRFSGSGSGTDFTLTISRLEPEDFAVYFCQHYGG | |
| | SPRPFGQGTKVEIKR | |
| | IgG domain-Linker-ScFv domain-DNA sequence | |
| | gaagtgcagctggtggaaagcggcggcggcctggtgcagccgggccgcagcctgcgcctg | SEQ ID |
| | agctgcgcggcgagcggctttacctttgatgatcatgcgatgttttgggtgcgccaggcg | NO: 155 |
| | ccgggcaaaggcctggaatgggtgagcggcattagctggagcagcaacagcattgcgtat | |
| | gcggatagcgtgaaaggccgctttaccattagccgcgataacgcgaaaaacagcctgtat | |
| | ctgcagatgaacagcctgcgcgcggaagataccgcggtgtattattgcgcgaaaggcagc | |
| | attgtggcgggcttttggctgggctattttgatctgtggggccgcggcaccctggtgacc | |
| | gtgagcagcgcgagcaccaaaggcccgagcgtgtttccgctggcgccgagcagcaaaagc | |
| | accagcggcggcaccgcggcgctgggctgcctggtgaaagattattttccggaaccggtg | |
| | accgtgagctggaacagcggcgcgctgaccagcggcgtgcatacctttccggcggtgctg | |
| | cagagcagcggcctgtatagcctgagcagcgtggtgaccgtgccgagcagcagcctgggc | |
| | acccagacctatatttgcaacgtgaaccataaaccgagcaacaccaaagtggataaaaaa | |
| | gcggaaccgaaaagctgcgataaaacccatacctgcccgccgtgcccggcgccggaagcg | |
| | gcgggcggcccgagcgtgtttctgtttccgccgaaaccgaaagataccctgatgattagc | |
| | cgcaccccggaagtgacctgcgtggtggtggatgtgagccatgaagatccggaagtgaaa | |
| | tttaactggtatgtggatggcgtggaagtgcataacgcgaaaaccaaaccgcgcgaagaa | |
| | cagtataacagcacctatcgcgtggtgagcgtgctgaccgtgctgcatcaggattggctg | |
| | aacggcaaagaatataaatgcaaagtgagcaacaaagcgctgccggcgccgattgaaaaa | |
| | accattagcaaagcgaaaggccagccgcgcgaaccgcaggtgtataccctgccgccgagc | |
| | cgcgatgaactgaccaaaaaccaggtgagcctgacctgcctggtgaaaggcttttatccg | |
| | agcgatattgcggtggaatgggaaagcaacggccagccggaaaacaactataaaaccacc | |
| | ccgccggtgctggatagcgatggcagcttttttctgtatagcaaactgaccgtggataaa | |
| | agccgctggcagcagggcaacgtgtttagctgcagcgtgatgcatgaagcgctgcataac | |
| | cattatacccagaaaagcctgagcctgagcccgggcggcggcggcggcagcggcggcggc | |
| | ggcagcggcggcggcggcagcggcggcggcggcagccaggtgcagctgcagcagagcgg | |
| | cccgggcctggtgaaaccgagccagaccctgagcctgacctgcgcgattagcggcgatagc | |
| | gtgagcagcaacagcgcggcgtggaactggattcgccagagcccgagccgcggcctggaa | |
| | tggctgggccgcacctattatcgcagcaaatggtataacgattatgcggtgagcgtgaaa | |
| | agccgcattaccattaacccggataccagcaaaaaccagtttagcctgcagatgaacagc | |
| | gtgaccccggaagataccgcgatttattattgcgcgagcgatgtggtgctggaaagcagc | |
| | agcagccgccattttgattattggggcaaaggcaccctggtgaccgtgagcagcggcggc | |
| | ggcggcagcggcggcggcggcagcggcggcggcggcagcggcggcggcggcagcgaaa | |
| | ttgtgctgacccagagcccgggcaccctgagcctgagcccgggcgaacgcgcgaccctgagc | |
| | tgccgcgcgagccagagcgtgagcagcagctatctggcgtggtatcagcagaaaccgggc | |
| | caggcgccgcgcctgctgatttatggcgcgagcagccgcgcgaccggcattccggatcgc | |
| | tttagcggcagcggcagcggcaccgattttaccctgaccattagccgcctggaaccggaa | |
| | gattttgcggtgtatttttgccagcattatggcggcagcccgcgcccgtttggccagggc | |
| | accaaagtggaaattaaacgc | |
| | IgG domain against HHLA2 | |
| | Heavy Chain | |
| | HCDR1 (IMGT) | SEQ ID |
| | GFTFDDHA | NO: 156 |
| | HCDR2 (IMGT) | SEQ ID |
| | ISWSSNSI | NO: 157 |
| | HCDR3 (IMGT) | SEQ ID |
| | AKGSIVAGFWLGYFDL | NO: 158 |
| | HCDR1 (Kabat) | SEQ ID |
| | DHAMF | NO: 159 |
| | HCDR2 (Kabat) | SEQ ID |
| | GISWSSNSIAYADSVKG | NO: 160 |
| | HCDR3 (Kabat) | SEQ ID |
| | GSIVAGFWLGYFDL | NO: 161 |
| | HCDR1 (Combined Kabat/IMGT) | SEQ ID |
| | GFTFDDHAMF | NO: 162 |
| | HCDR2 (Combined Kabat/IMGT) | SEQ ID |
| | GISWSSNSIAYADSVKG | NO: 163 |
| | HCDR3 (Combined Kabat/IMGT) | SEQ ID |
| | GSIVAGFWLGYFDL | NO: 164 |
| | VH-Amino acid sequence | SEQ ID |
| | EVQLVESGGGLVQPGRSLRLSCAASGFTFDDHAMFWVRQAP | NO: 165 |
| | GKGLEWVSGISWSSNSIAYADSVKGRFTISRDNAKNSLYLQ | |
| | MNSLRAEDTAVYYCAKGSIVAGFWLGYFDLWGRGTLVTVS | |
| | S | |
| | VH-DNA sequence | SEQ ID |
| | GAAGTGCAGCTGGTGGAAAGCGGCGGCGGCCTGGTGCAG | NO: 166 |
| | CCGGGCCGCAGCCTGCGCCTGAGCTGCGCGGCGAGCGGCT | |
| | TTACCTTTGATGATCATGCGATGTTTTGGGTGCGCCAGGC | |
| | GCCGGGCAAAGGCCTGGAATGGGTGAGCGGCATTAGCTG | |
| | GAGCAGCAACAGCATTGCGTATGCGGATAGCGTGAAAGG | |
| | CCGCTTTACCATTAGCCGCGATAACGCGAAAAACAGCCTG | |
| | TATCTGCAGATGAACAGCCTGCGCGCGGAAGATACCGCG | |
| | GTGTATTATTGCGCGAAAGGCAGCATTGTGGCGGGCTTTT | |
| | GGCTGGGCTATTTTGATCTGTGGGGCCGCGGCACCCTGGT | |
| | GACCGTGAGCAGC | |
| | Heavy chain-Amino acid sequence | SEQ ID |
| | EVQLVESGGGLVQPGRSLRLSCAASGFTFDDHAMFWVRQAP | NO: 167 |
| | GKGLEWVSGISWSSNSIAYADSVKGRFTISRDNAKNSLYLQ | |
| | MNSLRAEDTAVYYCAKGSIVAGFWLGYFDLWGRGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKAEPKSCDKTHTCPPCPAPEAAGGPSVFLF | |
| | PPKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSRDELTKNQVSLTC | |
| | LVKGFYPSDIAVEWESNGQPENNYKTTPPVLDSDGSFFLYSK | |
| | LTVDKSRWQQGNVFSCSVMHEALHNHYTQKSLSLSPG | |
| | Heavy chain-DNA sequence | SEQ ID |
| | GAAGTGCAGCTGGTGGAAAGCGGCGGCGGCCTGGTGCAG | NO: 168 |
| | CCGGGCCGCAGCCTGCGCCTGAGCTGCGCGGCGAGCGGCT | |
| | TTACCTTTGATGATCATGCGATGTTTTGGGTGCGCCAGGC | |
| | GCCGGGCAAAGGCCTGGAATGGGTGAGCGGCATTAGCTG | |
| | GAGCAGCAACAGCATTGCGTATGCGGATAGCGTGAAAGG | |
| | CCGCTTTACCATTAGCCGCGATAACGCGAAAAACAGCCTG | |
| | TATCTGCAGATGAACAGCCTGCGCGCGGAAGATACCGCG | |
| | GTGTATTATTGCGCGAAAGGCAGCATTGTGGCGGGCTTTT | |
| | GGCTGGGCTATTTTGATCTGTGGGGCCGCGGCACCCTGGT | |
| | GACCGTGAGCAGCGCGAGCACCAAAGGCCCGAGCGTGTT | |
| | TCCGCTGGCGCCGAGCAGCAAAAGCACCAGCGGCGGCAC | |
| | CGCGGCGCTGGGCTGCCTGGTGAAAGATTATTTTCCGGAA | |
| | CCGGTGACCGTGAGCTGGAACAGCGGCGCGCTGACCAGC | |
| | GGCGTGCATACCTTTCCGGCGGTGCTGCAGAGCAGCGGCC | |
| | TGTATAGCCTGAGCAGCGTGGTGACCGTGCCGAGCAGCAG | |
| | CCTGGGCACCCAGACCTATATTTGCAACGTGAACCATAAA | |
| | CCGAGCAACACCAAAGTGGATAAAAAAGCGGAACCGAAA | |
| | AGCTGCGATAAAACCCATACCTGCCCGCCGTGCCCGGCGC | |
| | CGGAAGCGGCGGGCGGCCCGAGCGTGTTTCTGTTTCCGCC | |
| | GAAACCGAAAGATACCCTGATGATTAGCCGCACCCCGGA | |
| | AGTGACCTGCGTGGTGGTGGATGTGAGCCATGAAGATCCG | |
| | GAAGTGAAATTTAACTGGTATGTGGATGGCGTGGAAGTGC | |
| | ATAACGCGAAAACCAAACCGCGCGAAGAACAGTATAACA | |
| | GCACCTATCGCGTGGTGAGCGTGCTGACCGTGCTGCATCA | |
| | GGATTGGCTGAACGGCAAAGAATATAAATGCAAAGTGAG | |
| | CAACAAAGCGCTGCCGGCGCCGATTGAAAAAACCATTAG | |
| | CAAAGCGAAAGGCCAGCCGCGCGAACCGCAGGTGTATAC | |
| | CCTGCCGCCGAGCCGCGATGAACTGACCAAAAACCAGGT | |
| | GAGCCTGACCTGCCTGGTGAAAGGCTTTTATCCGAGCGAT | |
| | ATTGCGGTGGAATGGGAAAGCAACGGCCAGCCGGAAAAC | |
| | AACTATAAAACCACCCCGCCGGTGCTGGATAGCGATGGCA | |
| | GCTTTTTTCTGTATAGCAAACTGACCGTGGATAAAAGCCG | |
| | CTGGCAGCAGGGCAACGTGTTTAGCTGCAGCGTGATGCAT | |
| | GAAGCGCTGCATAACCATTATACCCAGAAAAGCCTGAGCC | |
| | TGAGCCCGGGC | |
| | ScFv against TMIGD2 | |
| | VH-Linker-VL-Amino acid sequence | SEQ ID |
| | QVQLQQSGPGLVKPSQTLSLTCAISGDSVSSNSAAWNWIRQS | NO: 169 |
| | PSRGLEWLGRTYYRSKWYNDYAVSVKSRITINPDTSKNQFSL | |
| | QMNSVTPEDTAIYYCASDVVLESSSSRHFDYWGKGTLVTVSS | |
| | GGGGSGGGGSGGGGSGGGGSEIVLTQSPGTLSLSPGERATLS | |
| | CRASQSVSSSYLAWYQQKPGQAPRLLIYGASSRATGIPDRFSG | |
| | SGSGTDFTLTISRLEPEDFAVYFCQHYGGSPRPFGQGTKVEIK | |
| | R | |
| | VH-Linker-VL-DNA sequence | SEQ ID |
| | caggtgcagctgcagcagagcggcccgggcctggtgaaaccgagccagaccctgagcctg | NO: 170 |
| | acctgcgcgattagcggcgatagcgtgagcagcaacagcgcggcgtggaactggattcgc | |
| | cagagcccgagccgcggcctggaatggctgggccgcacctattatcgcagcaaatggtat | |
| | aacgattatgcggtgagcgtgaaaagccgcattaccattaacccggataccagcaaaaac | |
| | cagtttagcctgcagatgaacagcgtgaccccggaagataccgcgatttattattgcgcg | |
| | agcgatgtggtgctggaaagcagcagcagccgccattttgattattggggcaaaggcacc | |
| | ctggtgaccgtgagcagcggcggcggcggcagcggcggcggcggcagcggcggcggcgg | |
| | cagcggcggcggcggcagcgaaattgtgctgacccagagcccgggcaccctgagcctgagc | |
| | ccgggcgaacgcgcgaccctgagctgccgcgcgagccagagcgtgagcagcagctatctg | |
| | gcgtggtatcagcagaaaccgggccaggcgccgcgcctgctgatttatggcgcgagcagc | |
| | cgcgcgaccggcattccggatcgctttagcggcagcggcagcggcaccgattttaccctg | |
| | accattagccgcctggaaccggaagattttgcggtgtatttttgccagcattatggcggc | |
| | agcccgcgcccgtttggccagggcaccaaagtggaaattaaacgc | |
| | VH Domain | SEQ ID |
| | QVQLQQSGPGLVKPSQTLSLTCAISGDSVSSNSAAWNWIRQS | NO: 171 |
| | PSRGLEWLGRTYYRSKWYNDYAVSVKSRITINPDTSKNQFSL | |
| | QMNSVTPEDTAIYYCASDVVLESSSSRHFDYWGKGTLVTVS | |
| | S | |
| | HCDR1 (IMGT) | SEQ ID |
| | GDSVSSNSAA | NO: 172 |
| | HCDR2 (IMGT) | SEQ ID |
| | TYYRSKWYN | NO: 173 |
| | HCDR3 (IMGT) | SEQ ID |
| | ASDVVLESSSSRHFDY | NO: 174 |
| | HCDR1 (Kabat) | SEQ ID |
| | SNSAAWN | NO: 175 |
| | HCDR2 (Kabat) | SEQ ID |
| | RTYYRSKWYNDYAVSVKS | NO: 176 |
| | HCDR3 (Kabat) | SEQ ID |
| | DVVLESSSSRHFDY | NO: 177 |
| | HCDR1 (Combined Kabat/IMGT) | SEQ ID |
| | GDSVSSNSAAWN | NO: 178 |
| | HCDR2 (Combined Kabat/IMGT) | SEQ ID |
| | RTYYRSKWYNDYAVSVKS | NO: 179 |
| | HCDR3 (Combined Kabat/IMGT) | SEQ ID |
| | DVVLESSSSRHFDY | NO: 180 |
| | Linker | SEQ ID |
| | GGGGSGGGGSGGGGSGGGGS | NO: 181 |
| | VL domain | SEQ ID |
| | EIVLTQSPGTLSLSPGERATLSCRASQSVSSSYLAWYQQKPGQ | NO: 182 |
| | APRLLIYGASSRATGIPDRFSGSGSGTDFTLTISRLEPEDFAVYF | |
| | CQHYGGSPRPFGQGTKVEIKR | |
| | LCDR1 (IMGT) | SEQ ID |
| | QSVSSSY | NO: 183 |
| | LCDR2 (IMGT) | SEQ ID |
| | GA | NO: 184 |
| | LCDR3 (IMGT) | SEQ ID |
| | QHYGGSPRP | NO: 185 |
| | LCDR1 (Kabat) | SEQ ID |
| | RASQSVSSSYLA | NO: 186 |
| | LCDR2 (Kabat) | SEQ ID |
| | GASSRAT | NO: 187 |
| | LCDR3 (Kabat) | SEQ ID |
| | QHYGGSPRP | NO: 188 |
| | LCDR1 (Combined Kabat/IMGT) | SEQ ID |
| | RASQSVSSSYLA | NO: 189 |
| | LCDR2 (Combined Kabat/IMGT) | SEQ ID |
| | GASSRAT | NO: 190 |
| | LCDR3 (Combined Kabat/IMGT) | SEQ ID |
| | QHYGGSPRP | NO: 191 |
| | Light chain: IgG domain against HHLA2 | |
| | LCDR1 (IMGT) | SEQ ID |
| | QSVSSY | NO: 192 |
| | LCDR2 (IMGT) | SEQ ID |
| | DA | NO: 193 |
| | LCDR3 (IMGT) | SEQ ID |
| | QQVSGNPLWT | NO: 194 |
| | LCDR1 (Kabat) | SEQ ID |
| | RASQSVSSYLA | NO: 195 |
| | LCDR2 (Kabat) | SEQ ID |
| | DASNRAT | NO: 196 |
| | LCDR3 (Kabat) | SEQ ID |
| | QQVSGNPLWT | NO: 197 |
| | LCDR1 (Combined Kabat/IMGT) | SEQ ID |
| | RASQSVSSYLA | NO: 198 |
| | LCDR2 (Combined Kabat/IMGT) | SEQ ID |
| | DASNRAT | NO: 199 |
| | LCDR3 (Combined Kabat/IMGT) | SEQ ID |
| | QQVSGNPLWT | NO: 200 |
| | VL-Amino acid sequence | SEQ ID |
| | EIVLTQSPATLSLSPGERATLSCRASQSVSSYLAWYQQKPGQ | NO: 201 |
| | APRLLIYDASNRATGIPARFSGSGSGTDFTLTISSLEPEDFAVY | |
| | YCQQVSGNPLWTFGGGTKVEIK | |
| | VL-DNA sequence | SEQ ID |
| | GAAATTGTGCTGACCCAGAGCCCGGCGACCCTGAGCCTGA | NO: 202 |
| | GCCCGGGCGAACGCGCGACCCTGAGCTGCCGCGCGAGCC | |
| | AGAGCGTGAGCAGCTATCTGGCGTGGTATCAGCAGAAAC | |
| | CGGGCCAGGCGCCGCGCCTGCTGATTTATGATGCGAGCAA | |
| | CCGCGCGACCGGCATTCCGGCGCGCTTTAGCGGCAGCGGC | |
| | AGCGGCACCGATTTTACCCTGACCATTAGCAGCCTGGAAC | |
| | CGGAAGATTTTGCGGTGTATTATTGCCAGCAGGTGAGCGG | |
| | CAACCCGCTGTGGACCTTTGGCGGCGGCACCAAAGTGGAA | |
| | ATTAAA | |
| | Light chain-Amino acid sequence | SEQ ID |
| | EIVLTQSPATLSLSPGERATLSCRASQSVSSYLAWYQQKPGQ | NO: 203 |
| | APRLLIYDASNRATGIPARFSGSGSGTDFTLTISSLEPEDFAVY | |
| | YCQQVSGNPLWTFGGGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| | Light chain-DNA sequence | SEQ ID |
| | GAAATTGTGCTGACCCAGAGCCCGGCGACCCTGAGCCTGA | NO: 204 |
| | GCCCGGGCGAACGCGCGACCCTGAGCTGCCGCGCGAGCC | |
| | AGAGCGTGAGCAGCTATCTGGCGTGGTATCAGCAGAAAC | |
| | CGGGCCAGGCGCCGCGCCTGCTGATTTATGATGCGAGCAA | |
| | CCGCGCGACCGGCATTCCGGCGCGCTTTAGCGGCAGCGGC | |
| | AGCGGCACCGATTTTACCCTGACCATTAGCAGCCTGGAAC | |
| | CGGAAGATTTTGCGGTGTATTATTGCCAGCAGGTGAGCGG | |
| | CAACCCGCTGTGGACCTTTGGCGGCGGCACCAAAGTGGAA | |
| | ATTAAACGCACCGTGGCGGCGCCGAGCGTGTTTATTTTTC | |
| | CGCCGAGCGATGAACAGCTGAAAAGCGGCACCGCGAGCG | |
| | TGGTGTGCCTGCTGAACAACTTTTATCCGCGCGAAGCGAA | |
| | AGTGCAGTGGAAAGTGGATAACGCGCTGCAGAGCGGCAA | |
| | CAGCCAGGAAAGCGTGACCGAACAGGATAGCAAAGATAG | |
| | CACCTATAGCCTGAGCAGCACCCTGACCCTGAGCAAAGCG | |
| | GATTATGAAAAACATAAAGTGTATGCGTGCGAAGTGACCC | |
| | ATCAGGGCCTGAGCAGCCCGGTGACCAAAAGCTTTAACCG | |
| | CGGCGAATGC | |

TABLE 5
Amino acid and nucleotide sequences of exemplary bispecific antibody
molecules and antigen-binding fragments thereof.

| | | SEQ ID |
|---|---|---|
| Clone | Sequence | NO |
| TK20- | Heavy chain: IgG domain (heavy chain) against CD20 (Rituximab) + linker + | |
| 39C9-2134 | scFv against TMIGD2 | |
| | IgG domain-Linker-ScFv domain-Linker-ScFv domain- | SEQ ID |
| | Amino acid sequence | NO: 205 |
| | QVQLQQPGAELVKPGASVKMSCKASGYTFTSYNMHWVKQT | |
| | PGRGLEWIGAIYPGNGDTSYNQKFKGKATLTADKSSSTAYMQ | |
| | LSSLTSEDSAVYYCARSTYYGGDWYFNVWGAGTTVTVSAAS | |
| | TKGPSVFPLAPSSKSTSGGTAALGCLVKDYFPEPVTVSWNSG | |
| | ALTSGVHTFPAVLQSSGLYSLSSVVTVPSSSLGTQTYICNVNH | |
| | KPSNTKVDKKAEPKSCDKTHTCPPCPAPEAAGGPSVFLFPPK | |
| | PKDTLMISRTPEVTCVVVDVSHEDPEVKFNWYVDGVEVHNA | |
| | KTKPREEQYNSTYRVVSVLTVLHQDWLNGKEYKCKVSNKA | |
| | LPAPIEKTISKAKGQPREPQVYTLPPSRDELTKNQVSLTCLVK | |
| | GFYPSDIAVEWESNGQPENNYKTTPPVLDSDGSFFLYSKLTVD | |
| | KSRWQQGNVFSCSVMHEALHNHYTQKSLSLSPGGGGGSGG | |
| | GGSGGGGSGGGGSQVQLQESGPGLVKPSQTLSLTCTVTGYSI | |
| | TSDFAWNWLRQHPGKGLEWIGYINYAGSSSYNPSHKSRVTIS | |
| | RDTSKNQFFLKLSSVTAADTAVYYCARSNYRNDDAMDSWG | |
| | QGTTVTVSSGGGGSGGGGSGGGGSGGGGSDIQLTQSPSFLSA | |
| | SVGDRVTITCKASQDVGTAVAWYQQKPGKAPKLLIYWASTR | |
| | HTGVPSRFSGSGSGTDFTLTISSLQPEDFATYYCQQFSGFPWTF | |
| | GGGTKVEIK | |
| | IgG domain-Linker-ScFv domain-DNA sequence | SEQ ID |
| | CAGGTGCAGCTGCAGCAGCCGGGCGCGGAACTGGTGAAA | NO: 206 |
| | CCGGGCGCGAGCGTGAAAATGAGCTGCAAAGCGAGCGGC | |
| | TATACCTTTACCAGCTATAACATGCATTGGGTGAAACAGA | |
| | CCCCGGGCCGCGGCCTGGAATGGATTGGCGCGATTTATCC | |
| | GGGCAACGGCGATACCAGCTATAACCAGAAATTTAAAGG | |
| | CAAAGCGACCCTGACCGCGGATAAAAGCAGCAGCACCGC | |
| | GTATATGCAGCTGAGCAGCCTGACCAGCGAAGATAGCGC | |
| | GGTGTATTATTGCGCGCGCAGCACCTATTATGGCGGCGAT | |
| | TGGTATTTTAACGTGTGGGGCGCGGGCACCACCGTGACCG | |
| | TGAGCGCGGCGAGCACCAAAGGCCCGAGCGTGTTTCCGCT | |
| | GGCGCCGAGCAGCAAAAGCACCAGCGGCGGCACCGCGGC | |
| | GCTGGGCTGCCTGGTGAAAGATTATTTTCCGGAACCGGTG | |
| | ACCGTGAGCTGGAACAGCGGCGCGCTGACCAGCGGCGTG | |
| | CATACCTTTCCGGCGGTGCTGCAGAGCAGCGGCCTGTATA | |
| | GCCTGAGCAGCGTGGTGACCGTGCCGAGCAGCAGCCTGG | |
| | GCACCCAGACCTATATTTGCAACGTGAACCATAAACCGAG | |
| | CAACACCAAAGTGGATAAAAAAGCGGAACCGAAAAGCTG | |
| | CGATAAAACCCATACCTGCCCGCCGTGCCCGGCGCCGGAA | |
| | GCGGCGGGCGGCCCGAGCGTGTTTCTGTTTCCGCCGAAAC | |
| | CGAAAGATACCCTGATGATTAGCCGCACCCCGGAAGTGAC | |
| | CTGCGTGGTGGTGGATGTGAGCCATGAAGATCCGGAAGTG | |
| | AAATTTAACTGGTATGTGGATGGCGTGGAAGTGCATAACG | |
| | CGAAAACCAAACCGCGCGAAGAACAGTATAACAGCACCT | |
| | ATCGCGTGGTGAGCGTGCTGACCGTGCTGCATCAGGATTG | |
| | GCTGAACGGCAAAGAATATAAATGCAAAGTGAGCAACAA | |
| | AGCGCTGCCGGCGCCGATTGAAAAAACCATTAGCAAAGC | |
| | GAAAGGCCAGCCGCGCGAACCGCAGGTGTATACCCTGCC | |
| | GCCGAGCCGCGATGAACTGACCAAAAACCAGGTGAGCCT | |
| | GACCTGCCTGGTGAAAGGCTTTTATCCGAGCGATATTGCG | |
| | GTGGAATGGGAAAGCAACGGCCAGCCGGAAAACAACTAT | |
| | AAAACCACCCCGCCGGTGCTGGATAGCGATGGCAGCTTTT | |
| | TTCTGTATAGCAAACTGACCGTGGATAAAAGCCGCTGGCA | |
| | GCAGGGCAACGTGTTTAGCTGCAGCGTGATGCATGAAGCG | |
| | CTGCATAACCATTATACCCAGAAAAGCCTGAGCCTGAGCC | |
| | CGGGCGGCGGCGGCGGCAGCGGCGGCGGCGGCAGCGGCG | |
| | GCGGCGGCAGCGGCGGCGGCGGCAGCCAGGTGCAGCTGC | |
| | AGGAAAGCGGCCCGGGCCTGGTGAAACCGAGCCAGACCC | |
| | TGAGCCTGACCTGCACCGTGACCGGCTATAGCATTACCAG | |
| | CGATTTTGCGTGGAACTGGCTGCGCCAGCATCCGGGCAAA | |
| | GGCCTGGAATGGATTGGCTATATTAACTATGCGGGCAGCA | |
| | GCAGCTATAACCCGAGCCATAAAAGCCGCGTGACCATTAG | |
| | CCGCGATACCAGCAAAAACCAGTTTTTTCTGAAACTGAGC | |
| | AGCGTGACCGCGGCGGATACCGCGGTGTATTATTGCGCGC | |
| | GCAGCAACTATCGCAACGATGATGCGATGGATAGCTGGG | |
| | GCCAGGGCACCACCGTGACCGTGAGCAGCGGCGGCGGCG | |
| | GCAGCGGCGGCGGCGGCAGCGGCGGCGGCGGCAGCGGCG | |
| | GCGGCGGCAGCGATATTCAGCTGACCCAGAGCCCGAGCTT | |
| | TCTGAGCGCGAGCGTGGGCGATCGCGTGACCATTACCTGC | |
| | AAAGCGAGCCAGGATGTGGGCACCGCGGTGGCGTGGTAT | |
| | CAGCAGAAACCGGGCAAAGCGCCGAAACTGCTGATTTATT | |
| | GGGCGAGCACCCGCCATACCGGCGTGCCGAGCCGCTTTAG | |
| | CGGCAGCGGCAGCGGCACCGATTTTACCCTGACCATTAGC | |
| | AGCCTGCAGCCGGAAGATTTTGCGACCTATTATTGCCAGC | |
| | AGTTTAGCGGCTTTCCGTGGACCTTTGGCGGCGGCACCAA | |
| | AGTGGAAATTAAA | |
| | IgG domain against CD20 | |
| | Heavy chain-Amino acid sequence | SEQ ID |
| | QVQLQQPGAELVKPGASVKMSCKASGYTFTSYNMHWVKQT | NO: 207 |
| | PGRGLEWIGAIYPGNGDTSYNQKFKGKATLTADKSSSTAYM | |
| | QLSSLTSEDSAVYYCARSTYYGGDWYFNVWGAGTTVTVSA | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKAEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| | Light chain-Amino acid sequence | SEQ ID |
| | QIVLSQSPAILSASPGEKVTMTCRASSSVSYIHWFQQKPGSSP | NO: 208 |
| | KPWIYATSNLASGVPVRFSGSGSGTSYSLTISRVEAEDAATY | |
| | YCQQWTSNPPTFGGGTKLEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| | ScFv against TMIGD2 | |
| | VH-Linker-VL-Amino acid sequence | SEQ ID |
| | QVQLQESGPGLVKPSQTLSLTCTVTGYSITSDFAWNWLRQHP | NO: 209 |
| | GKGLEWIGYINYAGSSSYNPSHKSRVTISRDTSKNQFFLKLSS | |
| | VTAADTAVYYCARSNYRNDDAMDSWGQGTTVTVSSGGGGS | |
| | GGGGSGGGGSGGGGSDIQLTQSPSFLSASVGDRVTITCKASQ | |
| | DVGTAVAWYQQKPGKAPKLLIYWASTRHTGVPSRFSGSGSGT | |
| | DFTLTISSLQPEDFATYYCQQFSGFPWTFGGGTKVEIK | |
| | VH-Linker-VL-DNA sequence | SEQ ID |
| | CAGGTGCAGCTGCAGGAAAGCGGCCCGGGCCTGGTGAAA | NO: 210 |
| | CCGAGCCAGACCCTGAGCCTGACCTGCACCGTGACCGGCT | |
| | ATAGCATTACCAGCGATTTTGCGTGGAACTGGCTGCGCCA | |
| | GCATCCGGGCAAAGGCCTGGAATGGATTGGCTATATTAAC | |
| | TATGCGGGCAGCAGCAGCTATAACCCGAGCCATAAAAGC | |
| | CGCGTGACCATTAGCCGCGATACCAGCAAAAACCAGTTTT | |
| | TTCTGAAACTGAGCAGCGTGACCGCGGCGGATACCGCGGT | |
| | GTATTATTGCGCGCGCAGCAACTATCGCAACGATGATGCG | |
| | ATGGATAGCTGGGGCCAGGGCACCACCGTGACCGTGAGC | |
| | AGCGGCGGCGGCGGCAGCGGCGGCGGCGGCAGCGGCGGC | |
| | GGCGGCAGCGGCGGCGGCGGCAGCGATATTCAGCTGACC | |
| | CAGAGCCCGAGCTTTCTGAGCGCGAGCGTGGGCGATCGCG | |
| | TGACCATTACCTGCAAAGCGAGCCAGGATGTGGGCACCGC | |
| | GGTGGCGTGGTATCAGCAGAAACCGGGCAAAGCGCCGAA | |
| | ACTGCTGATTTATTGGGCGAGCACCCGCCATACCGGCGTG | |
| | CCGAGCCGCTTTAGCGGCAGCGGCAGCGGCACCGATTTTA | |
| | CCCTGACCATTAGCAGCCTGCAGCCGGAAGATTTTGCGAC | |
| | CTATTATTGCCAGCAGTTTAGCGGCTTTCCGTGGACCTTTG | |
| | GCGGCGGCACCAAAGTGGAAATTAAA | |
| | VH Domain | SEQ ID |
| | QVQLQESGPGLVKPSQTLSLTCTVTGYSITSDFAWNWLRQHP | NO: 211 |
| | GKGLEWIGYINYAGSSSYNPSHKSRVTISRDTSKNQFFLKLSS | |
| | VTAADTAVYYCARSNYRNDDAMDSWGQGTTVTVSS | |
| | HCDR1 (IMGT) | SEQ ID |
| | GYSITSDFA | NO: 212 |
| | HCDR2 (IMGT) | SEQ ID |
| | INYAGSS | NO: 213 |
| | HCDR3 (IMGT) | SEQ ID |
| | ARSNYRNDDAMDS | NO: 214 |
| | HCDR1 (Kabat) | SEQ ID |
| | SDFAWN | NO: 215 |
| | HCDR2 (Kabat) | SEQ ID |
| | YINYAGSSSYNPSHKS | NO: 216 |
| | HCDR3 (Kabat) | SEQ ID |
| | SNYRNDDAMDS | NO: 217 |
| | HCDR1 (Combined Kabat/IMGT) | SEQ ID |
| | GYSITSDFAWN | NO: 218 |
| | HCDR2 (Combined Kabat/IMGT) | SEQ ID |
| | YINYAGSSSYNPSHKS | NO: 219 |
| | HCDR3 (Combined Kabat/IMGT) | SEQ ID |
| | SNYRNDDAMDS | NO: 220 |
| | Linker | SEQ ID |
| | GGGGSGGGGSGGGGSGGGGS | NO: 221 |
| | VL domain | SEQ ID |
| | DIQLTQSPSFLSASVGDRVTITCKASQDVGTAVAWYQQKPGK | NO: 222 |
| | APKLLIYWASTRHTGVPSRFSGSGSGTDFTLTISSLQPEDFATY | |
| | YCQQFSGFPWTFGGGTKVEIK | |
| | LCDR1 (IMGT) | SEQ ID |
| | QDVGTA | NO: 223 |
| | LCDR2 (IMGT) | SEQ ID |
| | WA | NO: 224 |
| | LCDR3 (IMGT) | SEQ ID |
| | QQFSGFPWT | NO: 225 |
| | LCDR1 (Kabat) | SEQ ID |
| | KASQDVGTAVA | NO: 226 |
| | LCDR2 (Kabat) | SEQ ID |
| | WASTRHT | NO: 227 |
| | LCDR3 (Kabat) | SEQ ID |
| | QQFSGFPWT | NO: 228 |
| | LCDR1 (Combined Kabat/IMGT) | SEQ ID |
| | KASQDVGTAVA | NO: 229 |
| | LCDR2 (Combined Kabat/IMGT) | SEQ ID |
| | WASTRHT | NO: 230 |
| | LCDR3 (Combined Kabat/IMGT) | SEQ ID |
| | QQFSGFPWT | NO: 231 |
| | Light chain: IgG domain against CD20 | |
| | Light chain-Amino acid sequence | SEQ ID |
| | QIVLSQSPAILSASPGEKVTMTCRASSSVSYIHWFQQKPGSSP | NO: 232 |
| | KPWIYATSNLASGVPVRFSGSGSGTSYSLTISRVEAEDAATYY | |
| | CQQWTSNPPTFGGGTKLEIKRTVAAPSVFIFPPSDEQLKSGTA | |
| | SVVCLLNNFYPREAKVQWKVDNALQSGNSQESVTEQDSKD | |
| | STYSLSSTLTLSKADYEKHKVYACEVTHQGLSSPVTKSFNRG | |
| | EC | |
| | Light chain-DNA sequence | SEQ ID |
| | CAGATTGTGCTGAGCCAGAGCCCGGCGATTCTGAGCGCGA | NO: 233 |
| | GCCCGGGCGAAAAAGTGACCATGACCTGCCGCGCGAGCA | |
| | GCAGCGTGAGCTATATTCATTGGTTTCAGCAGAAACCGGG | |
| | CAGCAGCCCGAAACCGTGGATTTATGCGACCAGCAACCTG | |
| | GCGAGCGGCGTGCCGGTGCGCTTTAGCGGCAGCGGCAGC | |
| | GGCACCAGCTATAGCCTGACCATTAGCCGCGTGGAAGCGG | |
| | AAGATGCGGCGACCTATTATTGCCAGCAGTGGACCAGCAA | |
| | CCCGCCGACCTTTGGCGGCGGCACCAAACTGGAAATTAAA | |
| | CGCACCGTGGCGGCGCCGAGCGTGTTTATTTTTCCGCCGA | |
| | GCGATGAACAGCTGAAAAGCGGCACCGCGAGCGTGGTGT | |
| | GCCTGCTGAACAACTTTTATCCGCGCGAAGCGAAAGTGCA | |
| | GTGGAAAGTGGATAACGCGCTGCAGAGCGGCAACAGCCA | |
| | GGAAAGCGTGACCGAACAGGATAGCAAAGATAGCACCTA | |
| | TAGCCTGAGCAGCACCCTGACCCTGAGCAAAGCGGATTAT | |
| | GAAAAACATAAAGTGTATGCGTGCGAAGTGACCCATCAG | |
| | GGCCTGAGCAGCCCGGTGACCAAAAGCTTTAACCGCGGC | |
| | GAATGC | |
| TK20- | Heavy chain: IgG domain (heavy chain) against CD20 (Rituximab) + linker + | |
| 17C7-01 | scFv against TMIGD2 | |
| | IgG domain-Linker-ScFv domain-Linker-ScFv domain- | SEQ ID |
| | Amino acid sequence | NO: 234 |
| | QVQLQQPGAELVKPGASVKMSCKASGYTFTSYNMHWVKQT | |
| | PGRGLEWIGAIYPGNGDTSYNQKFKGKATLTADKSSSTAYMQ | |
| | LSSLTSEDSAVYYCARSTYYGGDWYFNVWGAGTTVTVSAAS | |
| | TKGPSVFPLAPSSKSTSGGTAALGCLVKDYFPEPVTVSWNSG | |
| | ALTSGVHTFPAVLQSSGLYSLSSVVTVPSSSLGTQTYICNVNH | |
| | KPSNTKVDKKAEPKSCDKTHTCPPCPAPEAAGGPSVFLFPPK | |
| | PKDTLMISRTPEVTCVVVDVSHEDPEVKFNWYVDGVEVHNA | |
| | KTKPREEQYNSTYRVVSVLTVLHQDWLNGKEYKCKVSNKA | |
| | LPAPIEKTISKAKGQPREPQVYTLPPSRDELTKNQVSLTCLVK | |
| | GFYPSDIAVEWESNGQPENNYKTTPPVLDSDGSFFLYSKLTVD | |
| | KSRWQQGNVFSCSVMHEALHNHYTQKSLSLSPGGGGGSGG | |
| | GGSGGGGSGGGGSQVQLVQSGAEVKKPGSSVKVSCKASGY | |
| | TFTSYDINWVRQAPGQGLEWMGWIYPGEGSTNYNEKFKGR | |
| | VTITADKSTSTAYMELSSLRSEDTAVYYCARRGLRYYFDYWG | |
| | QGTTVTVSSGGGGSGGGGSGGGGSGGGGSDIQMTQSPSSLS | |
| | ASVGDRVTITCRASQDIRNYLNWYQQKPGKVPKLLIYYTSRL | |
| | HSGVPSRFSGSGSGTDFTLTISSLQPEDVATYYCQQVNTLPWT | |
| | FGGGTKVEIK | |
| | IgG domain-Linker-ScFv domain-DNA sequence | SEQ ID |
| | CAGGTGCAGCTGCAGCAGCCGGGCGCGGAACTGGTGAAA | NO: 235 |
| | CCGGGCGCGAGCGTGAAAATGAGCTGCAAAGCGAGCGGC | |
| | TATACCTTTACCAGCTATAACATGCATTGGGTGAAACAGA | |
| | CCCCGGGCCGCGGCCTGGAATGGATTGGCGCGATTTATCC | |
| | GGGCAACGGCGATACCAGCTATAACCAGAAATTTAAAGG | |
| | CAAAGCGACCCTGACCGCGGATAAAAGCAGCAGCACCGC | |
| | GTATATGCAGCTGAGCAGCCTGACCAGCGAAGATAGCGC | |
| | GGTGTATTATTGCGCGCGCAGCACCTATTATGGCGGCGAT | |
| | TGGTATTTTAACGTGTGGGGCGCGGGCACCACCGTGACCG | |
| | TGAGCGCGGCGAGCACCAAAGGCCCGAGCGTGTTTCCGCT | |
| | GGCGCCGAGCAGCAAAAGCACCAGCGGCGGCACCGCGGC | |
| | GCTGGGCTGCCTGGTGAAAGATTATTTTCCGGAACCGGTG | |
| | ACCGTGAGCTGGAACAGCGGCGCGCTGACCAGCGGCGTG | |
| | CATACCTTTCCGGCGGTGCTGCAGAGCAGCGGCCTGTATA | |
| | GCCTGAGCAGCGTGGTGACCGTGCCGAGCAGCAGCCTGG | |
| | GCACCCAGACCTATATTTGCAACGTGAACCATAAACCGAG | |
| | CAACACCAAAGTGGATAAAAAAGCGGAACCGAAAAGCTG | |
| | CGATAAAACCCATACCTGCCCGCCGTGCCCGGCGCCGGAA | |
| | GCGGCGGGCGGCCCGAGCGTGTTTCTGTTTCCGCCGAAAC | |
| | CGAAAGATACCCTGATGATTAGCCGCACCCCGGAAGTGAC | |
| | CTGCGTGGTGGTGGATGTGAGCCATGAAGATCCGGAAGTG | |
| | AAATTTAACTGGTATGTGGATGGCGTGGAAGTGCATAACG | |
| | CGAAAACCAAACCGCGCGAAGAACAGTATAACAGCACCT | |
| | ATCGCGTGGTGAGCGTGCTGACCGTGCTGCATCAGGATTG | |
| | GCTGAACGGCAAAGAATATAAATGCAAAGTGAGCAACAA | |
| | AGCGCTGCCGGCGCCGATTGAAAAAACCATTAGCAAAGC | |
| | GAAAGGCCAGCCGCGCGAACCGCAGGTGTATACCCTGCC | |
| | GCCGAGCCGCGATGAACTGACCAAAAACCAGGTGAGCCT | |
| | GACCTGCCTGGTGAAAGGCTTTTATCCGAGCGATATTGCG | |
| | GTGGAATGGGAAAGCAACGGCCAGCCGGAAAACAACTAT | |
| | AAAACCACCCCGCCGGTGCTGGATAGCGATGGCAGCTTTT | |
| | TTCTGTATAGCAAACTGACCGTGGATAAAAGCCGCTGGCA | |
| | GCAGGGCAACGTGTTTAGCTGCAGCGTGATGCATGAAGCG | |
| | CTGCATAACCATTATACCCAGAAAAGCCTGAGCCTGAGCC | |
| | CGGGCGGCGGCGGCGGCAGCGGCGGCGGCGGCAGCGGCG | |
| | GCGGCGGCAGCGGCGGCGGCGGCAGCCAGGTGCAGCTGG | |
| | TGCAGAGCGGCGCGGAAGTGAAAAAACCGGGCAGCAGCG | |
| | TGAAAGTGAGCTGCAAAGCGAGCGGCTATACCTTTACCAG | |
| | CTATGATATTAACTGGGTGCGCCAGGCGCCGGGCCAGGGC | |
| | CTGGAATGGATGGGCTGGATTTATCCGGGCGAAGGCAGC | |
| | ACCAACTATAACGAAAAATTTAAAGGCCGCGTGACCATTA | |
| | CCGCGGATAAAAGCACCAGCACCGCGTATATGGAACTGA | |
| | GCAGCCTGCGCAGCGAAGATACCGCGGTGTATTATTGCGC | |
| | GCGCCGCGGCCTGCGCTATTATTTTGATTATTGGGGCCAG | |
| | GGCACCACCGTGACCGTGAGCAGCGGCGGCGGCGGCAGC | |
| | GGCGGCGGCGGCAGCGGCGGCGGCGGCAGCGGCGGCGGC | |
| | GGCAGCGATATTCAGATGACCCAGAGCCCGAGCAGCCTG | |
| | AGCGCGAGCGTGGGCGATCGCGTGACCATTACCTGCCGCG | |
| | CGAGCCAGGATATTCGCAACTATCTGAACTGGTATCAGCA | |
| | GAAACCGGGCAAAGTGCCGAAACTGCTGATTTATTATACC | |
| | AGCCGCCTGCATAGCGGCGTGCCGAGCCGCTTTAGCGGCA | |
| | GCGGCAGCGGCACCGATTTTACCCTGACCATTAGCAGCCT | |
| | GCAGCCGGAAGATGTGGCGACCTATTATTGCCAGCAGGTG | |
| | AACACCCTGCCGTGGACCTTTGGCGGCGGCACCAAAGTGG | |
| | AAATTAAA | |
| | IgG domain against CD20 | |
| | Heavy chain-Amino acid sequence | SEQ ID |
| | QVQLQQPGAELVKPGASVKMSCKASGYTFTSYNMHWVKQT | NO: 236 |
| | PGRGLEWIGAIYPGNGDTSYNQKFKGKATLTADKSSSTAYM | |
| | QLSSLTSEDSAVYYCARSTYYGGDWYFNVWGAGTTVTVSA | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKAEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| | Light chain-Amino acid sequence | SEQ ID |
| | QIVLSQSPAILSASPGEKVTMTCRASSSVSYIHWFQQKPGSSP | NO: 237 |
| | KPWIYATSNLASGVPVRFSGSGSGTSYSLTISRVEAEDAATY | |
| | YCQQWTSNPPTFGGGTKLEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| | ScFv against TMIGD2 | |
| | Heavy Chain | |
| | VH-Linker-VL-Amino acid sequence | SEQ ID |
| | QVQLVQSGAEVKKPGSSVKVSCKASGYTFTSYDINWVRQAP | NO: 238 |
| | GQGLEWMGWIYPGEGSTNYNEKFKGRVTITADKSTSTAYME | |
| | LSSLRSEDTAVYYCARRGLRYYFDYWGQGTTVTVSSGGGGS | |
| | GGGGSGGGGSGGGGSDIQMTQSPSSLSASVGDRVTITCRASQ | |
| | DIRNYLNWYQQKPGKVPKLLIYYTSRLHSGVPSRFSGSGSGT | |
| | DFTLTISSLQPEDVATYYCQQVNTLPWTFGGGTKVEIK | |
| | VH-Linker-VL-DNA sequence | SEQ ID |
| | CAGGTGCAGCTGGTGCAGAGCGGCGCGGAAGTGAAAAAA | NO: 239 |
| | CCGGGCAGCAGCGTGAAAGTGAGCTGCAAAGCGAGCGGC | |
| | TATACCTTTACCAGCTATGATATTAACTGGGTGCGCCAGG | |
| | CGCCGGGCCAGGGCCTGGAATGGATGGGCTGGATTTATCC | |
| | GGGCGAAGGCAGCACCAACTATAACGAAAAATTTAAAGG | |
| | CCGCGTGACCATTACCGCGGATAAAAGCACCAGCACCGC | |
| | GTATATGGAACTGAGCAGCCTGCGCAGCGAAGATACCGC | |
| | GGTGTATTATTGCGCGCGCCGCGGCCTGCGCTATTATTTTG | |
| | ATTATTGGGGCCAGGGCACCACCGTGACCGTGAGCAGCG | |
| | GCGGCGGCGGCAGCGGCGGCGGCGGCAGCGGCGGCGGCG | |
| | GCAGCGGCGGCGGCGGCAGCGATATTCAGATGACCCAGA | |
| | GCCCGAGCAGCCTGAGCGCGAGCGTGGGCGATCGCGTGA | |
| | CCATTACCTGCCGCGCGAGCCAGGATATTCGCAACTATCT | |
| | GAACTGGTATCAGCAGAAACCGGGCAAAGTGCCGAAACT | |
| | GCTGATTTATTATACCAGCCGCCTGCATAGCGGCGTGCCG | |
| | AGCCGCTTTAGCGGCAGCGGCAGCGGCACCGATTTTACCC | |
| | TGACCATTAGCAGCCTGCAGCCGGAAGATGTGGCGACCTA | |
| | TTATTGCCAGCAGGTGAACACCCTGCCGTGGACCTTTGGC | |
| | GGCGGCACCAAAGTGGAAATTAAA | |
| | VH Domain | SEQ ID |
| | QVQLVQSGAEVKKPGSSVKVSCKASGYTFTSYDINWVRQAP | NO: 240 |
| | GQGLEWMGWIYPGEGSTNYNEKFKGRVTITADKSTSTAYM | |
| | ELSSLRSEDTAVYYCARRGLRYYFDYWGQGTTVTVSS | |
| | HCDR1 (IMGT) | SEQ ID |
| | GYTFTSYD | NO: 241 |
| | HCDR2 (IMGT) | SEQ ID |
| | IYPGEGST | NO: 242 |
| | HCDR3 (IMGT) | SEQ ID |
| | ARRGLRYYFDY | NO: 243 |
| | HCDR1 (Kabat) | SEQ ID |
| | SYDIN | NO: 244 |
| | HCDR2 (Kabat) | SEQ ID |
| | WIYPGEGSTNYNEKFKG | NO: 245 |
| | HCDR3 (Kabat) | SEQ ID |
| | RGLRYYFDY | NO: 246 |
| | HCDR1 (Combined Kabat/IMGT) | SEQ ID |
| | GYTFTSYDIN | NO: 247 |
| | HCDR2 (Combined Kabat/IMGT) | SEQ ID |
| | WIYPGEGSTNYNEKFKG | NO: 248 |
| | HCDR3 (Combined Kabat/IMGT) | SEQ ID |
| | RGLRYYFDY | NO: 249 |
| | Linker | SEQ ID |
| | GGGGSGGGGSGGGGSGGGGS | NO: 250 |
| | VL domain | SEQ ID |
| | DIQMTQSPSSLSASVGDRVTITCRASQDIRNYLNWYQQKPGK | NO: 251 |
| | VPKLLIYYTSRLHSGVPSRFSGSGSGTDFTLTISSLQPEDVATY | |
| | YCQQVNTLPWTFGGGTKVEIK | |
| | LCDR1 (IMGT) | SEQ ID |
| | QDIRNY | NO: 252 |
| | LCDR2 (IMGT) | SEQ ID |
| | YT | NO: 253 |
| | LCDR3 (IMGT) | SEQ ID |
| | QQVNTLPWT | NO: 254 |
| | LCDR1 (Kabat) | SEQ ID |
| | RASQDIRNYLN | NO: 255 |
| | LCDR2 (Kabat) | SEQ ID |
| | YTSRLHS | NO: 256 |
| | LCDR3 (Kabat) | SEQ ID |
| | QQVNTLPWT | NO: 257 |
| | LCDR1 (Combined Kabat/IMGT) | SEQ ID |
| | RASQDIRNYLN | NO: 258 |
| | LCDR2 (Combined Kabat/IMGT) | SEQ ID |
| | YTSRLHS | NO: 259 |
| | LCDR3 (Combined Kabat/IMGT) | SEQ ID |
| | QQVNTLPWT | NO: 260 |
| | Light chain: IgG domain against CD20 | |
| | Light chain-Amino acid sequence | SEQ ID |
| | QIVLSQSPAILSASPGEKVTMTCRASSSVSYIHWFQQKPGSSP | NO: 261 |
| | KPWIYATSNLASGVPVRFSGSGSGTSYSLTISRVEAEDAATYY | |
| | CQQWTSNPPTFGGGTKLEIKRTVAAPSVFIFPPSDEQLKSGTA | |
| | SVVCLLNNFYPREAKVQWKVDNALQSGNSQESVTEQDSKD | |
| | STYSLSSTLTLSKADYEKHKVYACEVTHQGLSSPVTKSFNRG | |
| | EC | |
| | Light chain-DNA sequence | SEQ ID |
| | CAGATTGTGCTGAGCCAGAGCCCGGCGATTCTGAGCGCGA | NO: 262 |
| | GCCCGGGCGAAAAAGTGACCATGACCTGCCGCGCGAGCA | |
| | GCAGCGTGAGCTATATTCATTGGTTTCAGCAGAAACCGGG | |
| | CAGCAGCCCGAAACCGTGGATTTATGCGACCAGCAACCTG | |
| | GCGAGCGGCGTGCCGGTGCGCTTTAGCGGCAGCGGCAGC | |
| | GGCACCAGCTATAGCCTGACCATTAGCCGCGTGGAAGCGG | |
| | AAGATGCGGCGACCTATTATTGCCAGCAGTGGACCAGCAA | |
| | CCCGCCGACCTTTGGCGGCGGCACCAAACTGGAAATTAAA | |
| | CGCACCGTGGCGGCGCCGAGCGTGTTTATTTTTCCGCCGA | |
| | GCGATGAACAGCTGAAAAGCGGCACCGCGAGCGTGGTGT | |
| | GCCTGCTGAACAACTTTTATCCGCGCGAAGCGAAAGTGCA | |
| | GTGGAAAGTGGATAACGCGCTGCAGAGCGGCAACAGCCA | |
| | GGAAAGCGTGACCGAACAGGATAGCAAAGATAGCACCTA | |
| | TAGCCTGAGCAGCACCCTGACCCTGAGCAAAGCGGATTAT | |
| | GAAAAACATAAAGTGTATGCGTGCGAAGTGACCCATCAG | |
| | GGCCTGAGCAGCCCGGTGACCAAAAGCTTTAACCGCGGC | |
| | GAATGC | |
| TK20- | Heavy chain: IgG domain (heavy chain) against CD20 (Rituximab) + linker + | |
| 29F8-16 | scFv against TMIGD2 | |
| | IgG domain-Linker-ScFv domain-Linker-ScFv domain- | SEQ ID |
| | Amino acid sequence | NO: 263 |
| | QVQLQQPGAELVKPGASVKMSCKASGYTFTSYNMHWVKQT | |
| | PGRGLEWIGAIYPGNGDTSYNQKFKGKATLTADKSSSTAYMQ | |
| | LSSLTSEDSAVYYCARSTYYGGDWYFNVWGAGTTVTVSAAS | |
| | TKGPSVFPLAPSSKSTSGGTAALGCLVKDYFPEPVTVSWNSG | |
| | ALTSGVHTFPAVLQSSGLYSLSSVVTVPSSSLGTQTYICNVNH | |
| | KPSNTKVDKKAEPKSCDKTHTCPPCPAPEAAGGPSVFLFPPK | |
| | PKDTLMISRTPEVTCVVVDVSHEDPEVKFNWYVDGVEVHNA | |
| | KTKPREEQYNSTYRVVSVLTVLHQDWLNGKEYKCKVSNKA | |
| | LPAPIEKTISKAKGQPREPQVYTLPPSRDELTKNQVSLTCLVK | |
| | GFYPSDIAVEWESNGQPENNYKTTPPVLDSDGSFFLYSKLTVD | |
| | KSRWQQGNVFSCSVMHEALHNHYTQKSLSLSPGGGGGSGG | |
| | GGSGGGGSGGGGSEVQLVESGGGLVKPGGSLRLSCAASGFTF | |
| | SSYAMSWVRQAPGKGLEWVSSISQGGGTYSPDSVKGRFTISR | |
| | DNAKNSLYLQMNSLRAEDTAVYYCARGADYYGTSWFGNW | |
| | GQGTLVTVSSGGGGSGGGGSGGGGSGGGGSDIVMTQSPLSL | |
| | PVTPGEPASISCRSGQSLVHSNANTYLHWYLQKPGQSPQLLIY | |
| | KVSNRFSGVPDRFSGSGSGTDFTLKISRVEAEDVGVYYCSQS | |
| | THVPWTFGGGTKVEIK | |
| | IgG domain-Linker-ScFv domain-DNA sequence | SEQ ID |
| | CAGGTGCAGCTGCAGCAGCCGGGCGCGGAACTGGTGAAA | NO: 264 |
| | CCGGGCGCGAGCGTGAAAATGAGCTGCAAAGCGAGCGGC | |
| | TATACCTTTACCAGCTATAACATGCATTGGGTGAAACAGA | |
| | CCCCGGGCCGCGGCCTGGAATGGATTGGCGCGATTTATCC | |
| | GGGCAACGGCGATACCAGCTATAACCAGAAATTTAAAGG | |
| | CAAAGCGACCCTGACCGCGGATAAAAGCAGCAGCACCGC | |
| | GTATATGCAGCTGAGCAGCCTGACCAGCGAAGATAGCGC | |
| | GGTGTATTATTGCGCGCGCAGCACCTATTATGGCGGCGAT | |
| | TGGTATTTTAACGTGTGGGGCGCGGGCACCACCGTGACCG | |
| | TGAGCGCGGCGAGCACCAAAGGCCCGAGCGTGTTTCCGCT | |
| | GGCGCCGAGCAGCAAAAGCACCAGCGGCGGCACCGCGGC | |
| | GCTGGGCTGCCTGGTGAAAGATTATTTTCCGGAACCGGTG | |
| | ACCGTGAGCTGGAACAGCGGCGCGCTGACCAGCGGCGTG | |
| | CATACCTTTCCGGCGGTGCTGCAGAGCAGCGGCCTGTATA | |
| | GCCTGAGCAGCGTGGTGACCGTGCCGAGCAGCAGCCTGG | |
| | GCACCCAGACCTATATTTGCAACGTGAACCATAAACCGAG | |
| | CAACACCAAAGTGGATAAAAAAGCGGAACCGAAAAGCTG | |
| | CGATAAAACCCATACCTGCCCGCCGTGCCCGGCGCCGGAA | |
| | GCGGCGGGCGGCCCGAGCGTGTTTCTGTTTCCGCCGAAAC | |
| | CGAAAGATACCCTGATGATTAGCCGCACCCCGGAAGTGAC | |
| | CTGCGTGGTGGTGGATGTGAGCCATGAAGATCCGGAAGTG | |
| | AAATTTAACTGGTATGTGGATGGCGTGGAAGTGCATAACG | |
| | CGAAAACCAAACCGCGCGAAGAACAGTATAACAGCACCT | |
| | ATCGCGTGGTGAGCGTGCTGACCGTGCTGCATCAGGATTG | |
| | GCTGAACGGCAAAGAATATAAATGCAAAGTGAGCAACAA | |
| | AGCGCTGCCGGCGCCGATTGAAAAAACCATTAGCAAAGC | |
| | GAAAGGCCAGCCGCGCGAACCGCAGGTGTATACCCTGCC | |
| | GCCGAGCCGCGATGAACTGACCAAAAACCAGGTGAGCCT | |
| | GACCTGCCTGGTGAAAGGCTTTTATCCGAGCGATATTGCG | |
| | GTGGAATGGGAAAGCAACGGCCAGCCGGAAAACAACTAT | |
| | AAAACCACCCCGCCGGTGCTGGATAGCGATGGCAGCTTTT | |
| | TTCTGTATAGCAAACTGACCGTGGATAAAAGCCGCTGGCA | |
| | GCAGGGCAACGTGTTTAGCTGCAGCGTGATGCATGAAGCG | |
| | CTGCATAACCATTATACCCAGAAAAGCCTGAGCCTGAGCC | |
| | CGGGCGGCGGCGGCGGCAGCGGCGGCGGCGGCAGCGGCG | |
| | GCGGCGGCAGCGGCGGCGGCGGCAGCGAAGTGCAGCTGG | |
| | TGGAAAGCGGCGGCGGCCTGGTGAAACCGGGCGGCAGCC | |
| | TGCGCCTGAGCTGCGCGGCGAGCGGCTTTACCTTTAGCAG | |
| | CTATGCGATGAGCTGGGTGCGCCAGGCGCCGGGCAAAGG | |
| | CCTGGAATGGGTGAGCAGCATTAGCCAGGGCGGCGGCAC | |
| | CTATAGCCCGGATAGCGTGAAAGGCCGCTTTACCATTAGC | |
| | CGCGATAACGCGAAAAACAGCCTGTATCTGCAGATGAAC | |
| | AGCCTGCGCGCGGAAGATACCGCGGTGTATTATTGCGCGC | |
| | GCGGCGCGGATTATTATGGCACCAGCTGGTTTGGCAACTG | |
| | GGGCCAGGGCACCCTGGTGACCGTGAGCAGCGGCGGCGG | |
| | CGGCAGCGGCGGCGGCGGCAGCGGCGGCGGCGGCAGCGG | |
| | CGGCGGCGGCAGCGATATTGTGATGACCCAGAGCCCGCTG | |
| | AGCCTGCCGGTGACCCCGGGCGAACCGGCGAGCATTAGCT | |
| | GCCGCAGCGGCCAGAGCCTGGTGCATAGCAACGCGAACA | |
| | CCTATCTGCATTGGTATCTGCAGAAACCGGGCCAGAGCCC | |
| | GCAGCTGCTGATTTATAAAGTGAGCAACCGCTTTAGCGGC | |
| | GTGCCGGATCGCTTTAGCGGCAGCGGCAGCGGCACCGATT | |
| | TTACCCTGAAAATTAGCCGCGTGGAAGCGGAAGATGTGG | |
| | GCGTGTATTATTGCAGCCAGAGCACCCATGTGCCGTGGAC | |
| | CTTTGGCGGCGGCACCAAAGTGGAAATTAAA | |
| | IgG domain against CD20 | |
| | Heavy chain-Amino acid sequence | SEQ ID |
| | QVQLQQPGAELVKPGASVKMSCKASGYTFTSYNMHWVKQT | NO: 265 |
| | PGRGLEWIGAIYPGNGDTSYNQKFKGKATLTADKSSSTAYM | |
| | QLSSLTSEDSAVYYCARSTYYGGDWYFNVWGAGTTVTVSA | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKAEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| | Light chain-Amino acid sequence | SEQ ID |
| | QIVLSQSPAILSASPGEKVTMTCRASSSVSYIHWFQQKPGSSP | NO: 266 |
| | KPWIYATSNLASGVPVRFSGSGSGTSYSLTISRVEAEDAATY | |
| | YCQQWTSNPPTFGGGTKLEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| | ScFv against TMIGD2 | |
| | Heavy Chain | |
| | VH-Linker-VL-Amino acid sequence | SEQ ID |
| | EVQLVESGGGLVKPGGSLRLSCAASGFTFSSYAMSWVRQAP | NO: 267 |
| | GKGLEWVSSISQGGGTYSPDSVKGRFTISRDNAKNSLYLQMN | |
| | SLRAEDTAVYYCARGADYYGTSWFGNWGQGTLVTVSSGGG | |
| | GSGGGGSGGGGSGGGGSDIVMTQSPLSLPVTPGEPASISCRSG | |
| | QSLVHSNANTYLHWYLQKPGQSPQLLIYKVSNRFSGVPDRFS | |
| | GSGSGTDFTLKISRVEAEDVGVYYCSQSTHVPWTFGGGTKV | |
| | EIK | |
| | VH-Linker-VL-DNA sequence | SEQ ID |
| | GAAGTGCAGCTGGTGGAAAGCGGCGGCGGCCTGGTGAAA | NO: 268 |
| | CCGGGCGGCAGCCTGCGCCTGAGCTGCGCGGCGAGCGGC | |
| | TTTACCTTTAGCAGCTATGCGATGAGCTGGGTGCGCCAGG | |
| | CGCCGGGCAAAGGCCTGGAATGGGTGAGCAGCATTAGCC | |
| | AGGGCGGCGGCACCTATAGCCCGGATAGCGTGAAAGGCC | |
| | GCTTTACCATTAGCCGCGATAACGCGAAAAACAGCCTGTA | |
| | TCTGCAGATGAACAGCCTGCGCGCGGAAGATACCGCGGT | |
| | GTATTATTGCGCGCGCGGCGCGGATTATTATGGCACCAGC | |
| | TGGTTTGGCAACTGGGGCCAGGGCACCCTGGTGACCGTGA | |
| | GCAGCGGCGGCGGCGGCAGCGGCGGCGGCGGCAGCGGCG | |
| | GCGGCGGCAGCGGCGGCGGCGGCAGCGATATTGTGATGA | |
| | CCCAGAGCCCGCTGAGCCTGCCGGTGACCCCGGGCGAACC | |
| | GGCGAGCATTAGCTGCCGCAGCGGCCAGAGCCTGGTGCAT | |
| | AGCAACGCGAACACCTATCTGCATTGGTATCTGCAGAAAC | |
| | CGGGCCAGAGCCCGCAGCTGCTGATTTATAAAGTGAGCAA | |
| | CCGCTTTAGCGGCGTGCCGGATCGCTTTAGCGGCAGCGGC | |
| | AGCGGCACCGATTTTACCCTGAAAATTAGCCGCGTGGAAG | |
| | CGGAAGATGTGGGCGTGTATTATTGCAGCCAGAGCACCCA | |
| | TGTGCCGTGGACCTTTGGCGGCGGCACCAAAGTGGAAATT | |
| | AAA | |
| | VH Domain | SEQ ID |
| | EVQLVESGGGLVKPGGSLRLSCAASGFTFSSYAMSWVRQAP | NO: 269 |
| | GKGLEWVSSISQGGGTYSPDSVKGRFTISRDNAKNSLYLQM | |
| | NSLRAEDTAVYYCARGADYYGTSWFGNWGQGTLVTVSS | |
| | HCDR1 (IMGT) | SEQ ID |
| | GFTFSSYA | NO: 270 |
| | HCDR2 (IMGT) | SEQ ID |
| | ISQGGGT | NO: 271 |
| | HCDR3 (IMGT) | SEQ ID |
| | ARGADYYGTSWFGN | NO: 272 |
| | HCDR1 (Kabat) | SEQ ID |
| | SYAMS | NO: 273 |
| | HCDR2 (Kabat) | SEQ ID |
| | SISQGGGTYSPDSVKG | NO: 274 |
| | HCDR3 (Kabat) | SEQ ID |
| | GADYYGTSWFGN | NO: 275 |
| | HCDR1 (Combined Kabat/IMGT) | SEQ ID |
| | GFTFSSYAMS | NO: 276 |
| | HCDR2 (Combined Kabat/IMGT) | SEQ ID |
| | ISQGGGTYSPDSVKG | NO: 277 |
| | HCDR3 (Combined Kabat/IMGT) | SEQ ID |
| | GADYYGTSWFGN | NO: 278 |
| | Linker | SEQ ID |
| | GGGGSGGGGSGGGGSGGGGS | NO: 279 |
| | VL domain | SEQ ID |
| | DIVMTQSPLSLPVTPGEPASISCRSGQSLVHSNANTYLHWYL | NO: 280 |
| | QKPGQSPQLLIYKVSNRFSGVPDRFSGSGSGTDFTLKISRVEA | |
| | EDVGVYYCSQSTHVPWTFGGGTKVEIK | |
| | LCDR1 (IMGT) | SEQ ID |
| | QSLVHSNANTY | NO: 281 |
| | LCDR2 (IMGT) | SEQ ID |
| | KV | NO: 282 |
| | LCDR3 (IMGT) | SEQ ID |
| | SQSTHVPWT | NO: 283 |
| | LCDR1 (Kabat) | SEQ ID |
| | RSGQSLVHSNANTYLH | NO: 284 |
| | LCDR2 (Kabat) | SEQ ID |
| | KVSNRFS | NO: 285 |
| | LCDR3 (Kabat) | SEQ ID |
| | SQSTHVPWT | NO: 286 |
| | LCDR1 (Combined Kabat/IMGT) | SEQ ID |
| | RSGQSLVHSNANTY | NO: 287 |
| | LCDR2 (Combined Kabat/IMGT) | SEQ ID |
| | KVSNRFS | NO: 288 |
| | LCDR3 (Combined Kabat/IMGT) | SEQ ID |
| | SQSTHVPWT | NO: 289 |
| | Light chain: IgG domain against CD20 | |
| | Light chain-Amino acid sequence | SEQ ID |
| | QIVLSQSPAILSASPGEKVTMTCRASSSVSYIHWFQQKPGSSP | NO: 290 |
| | KPWIYATSNLASGVPVRFSGSGSGTSYSLTISRVEAEDAATYY | |
| | CQQWTSNPPTFGGGTKLEIKRTVAAPSVFIFPPSDEQLKSGTA | |
| | SVVCLLNNFYPREAKVQWKVDNALQSGNSQESVTEQDSKD | |
| | STYSLSSTLTLSKADYEKHKVYACEVTHQGLSSPVTKSFNRG | |
| | EC | |
| | Light chain-DNA sequence | SEQ ID |
| | CAGATTGTGCTGAGCCAGAGCCCGGCGATTCTGAGCGCGA | NO: 291 |
| | GCCCGGGCGAAAAAGTGACCATGACCTGCCGCGCGAGCA | |
| | GCAGCGTGAGCTATATTCATTGGTTTCAGCAGAAACCGGG | |
| | CAGCAGCCCGAAACCGTGGATTTATGCGACCAGCAACCTG | |
| | GCGAGCGGCGTGCCGGTGCGCTTTAGCGGCAGCGGCAGC | |
| | GGCACCAGCTATAGCCTGACCATTAGCCGCGTGGAAGCGG | |
| | AAGATGCGGCGACCTATTATTGCCAGCAGTGGACCAGCAA | |
| | CCCGCCGACCTTTGGCGGCGGCACCAAACTGGAAATTAAA | |
| | CGCACCGTGGCGGCGCCGAGCGTGTTTATTTTTCCGCCGA | |
| | GCGATGAACAGCTGAAAAGCGGCACCGCGAGCGTGGTGT | |
| | GCCTGCTGAACAACTTTTATCCGCGCGAAGCGAAAGTGCA | |
| | GTGGAAAGTGGATAACGCGCTGCAGAGCGGCAACAGCCA | |
| | GGAAAGCGTGACCGAACAGGATAGCAAAGATAGCACCTA | |
| | TAGCCTGAGCAGCACCCTGACCCTGAGCAAAGCGGATTAT | |
| | GAAAAACATAAAGTGTATGCGTGCGAAGTGACCCATCAG | |
| | GGCCTGAGCAGCCCGGTGACCAAAAGCTTTAACCGCGGC | |
| | GAATGC | |

An antibody molecule or antigen-binding fragment thereof of a bispecific antibody molecule or antigen-binding fragment thereof described herein can be from any class of antibodies including, but not limited to, IgG, IgA, IgM, IgD, and IgE, and from any subclass (e.g., IgG1, IgG2, IgG3, and IgG4) of antibodies. An antibody molecule or antigen-binding fragment thereof described herein can be or comprise a human, humanized, CDR-grafted, or in vitro generated antibody. An antibody molecule or antigen-binding fragment thereof described herein can have or comprise a heavy chain constant region chosen from, e.g., IgG1, IgG2, IgG3, or IgG4. An antibody molecule or antigen-binding fragment thereof described herein can have or comprise a light chain chosen from, e.g., kappa or lambda.

An antibody molecule or antigen-binding fragment thereof of a bispecific antibody molecule or antigen-binding fragment thereof described herein can be or comprise a monoclonal antibody. In some embodiments, monoclonal antibodies directed to a particular epitope are derived from a single cell line (e.g., a B cell line).

In some embodiments, an antibody molecule or antigen-binding fragment thereof of a bispecific antibody molecule or antigen-binding fragment thereof described herein is or comprises a polyclonal antibody. In contrast to monoclonal antibodies, polyclonal antibodies are typically obtained from a population of heterogeneous antibodies, such that antibodies in a particular population include structural variation, for example, affinity for different epitopes on a particular target (e.g., TMIGD2 or a TAA described herein). Several methods of producing polyclonal antibodies are known and include, for example, use of multiple subcutaneous and/or intraperitoneal injections of a relevant antigen into an animal, optionally including co-administration of one or more adjuvants.

Many multispecific antibody constructs are known in the art, and the bispecific antibody molecules or antigen-binding fragments thereof provided herein may be provided in the form of any suitable multispecific construct. In some embodiments, bispecific antibody molecules or antigen-binding fragments thereof provided herein are multispecific antibody molecules or antigen-binding fragments thereof. In some embodiments, a multispecific antibody molecule or antigen-binding fragment thereof described herein binds more than one antigen, e.g., at least two antigens, at least three antigens, at least four antigens, or at least five antigens. In some embodiments, a multispecific antibody molecule or antigen-binding fragment thereof described herein binds more than one epitope on TMIGD2, e.g., at least two or at least three epitopes on TMIGD2. In some embodiments, a multispecific antibody molecule or antigen-binding fragment thereof described herein binds more than one epitope on a TAA described herein (e.g., HHLA2), e.g., at least two or at least three epitopes on a TAA described herein (e.g., HHLA2). In some embodiments, a multispecific antibody molecule or antigen-binding fragment thereof described herein binds two or more TAAs.

A bispecific antibody molecule or antigen-binding fragment thereof described herein can comprise or be a first antibody or fragment thereof attached to a N-terminus or C-terminus of a heavy or light chain of a second antibody (see, e.g., Coloma and Morrison, Nature Biotechnol., 1997, 15:159-163; incorporated by reference in its entirety). In some embodiments, a bispecific antibody molecule or antigen-binding fragment thereof described herein comprises or is a tetravalent bispecific antibody molecule. In some embodiments, a bispecific antibody molecule or antigen-binding fragment thereof described herein comprises or is a scFv attached to a C-terminus of a heavy chain of a full-length antibody (e.g., an IgG). In some embodiments, a bispecific antibody molecule or antigen-binding fragment thereof described herein comprises or is a hybrid immunoglobulin comprising at least two different heavy chain variable regions and at least two different light chain variable regions (see, e.g., Milstein and Cuello, Nature, 1983, 305:537-540; and Staerz and Bevan, Proc. Natl. Acad. Sci. USA, 1986, 83:1453-1457; each of which is incorporated by reference in its entirety).

In some embodiments, a bispecific antibody molecule or antigen-binding fragment thereof described herein comprises or is one or more immunoglobulin chains with alterations to reduce formation of side products that do not have multispecificity. In some embodiments, a bispecific antibody molecule or antigen-binding fragment thereof described herein comprise one or more “knobs-into-holes” modifications (see, e.g., U.S. Pat. No. 5,731,168; hereby incorporated by reference in its entirety).

In some embodiments, a bispecific antibody molecule or antigen-binding fragment thereof described herein comprises or is two half antibodies comprising a first half antibody having binding specificity for a first epitope and a second half antibody having binding specificity for a second epitope. In some embodiments, a bispecific antibody molecule or antigen-binding fragment thereof described herein comprises or is two scFvs comprising a first scFv have binding specificity for a first epitope and a second scFv have binding specificity for a second epitope. In some embodiments, a bispecific antibody molecule or antigen-binding fragment thereof described herein comprises or is a bispecific single chain molecule (see, e.g., Traunecker et al., EMBO J., 1991, 10:3655-3659; and Gruber et al., J. Immunol., 1994, 152:5368-5374; each of which is incorporated by reference in its entirety).

A bispecific antibody molecule or antigen-binding fragment thereof described herein can comprise a linker connecting a first antigen-binding domain and a second antigen-binding domain. In some embodiments, a linker comprises or is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. Typically, a linker is characterized in that it tends not to adopt a rigid three-dimensional structure, but rather provides flexibility to a polypeptide (e.g., a bispecific antibody molecule or antigen-binding fragment thereof described herein). A linker can be employed in a bispecific antibody molecule or antigen-binding fragment thereof described herein based on specific properties imparted to the bispecific antibody molecule or antigen-binding fragment thereof, such as, for example, a reduction in aggregation and/or an increase in stability. In some embodiments, a bispecific antibody molecule or antigen-binding fragment thereof described herein comprises one or more G4S linkers. In some embodiments, a bispecific antibody molecule or antigen-binding fragment thereof described herein comprises one or more (G4S) n linkers, wherein n is 1, 2, 3, 4, 5, 6, 7, 8, 9, 10, 11, 12, 13, 14, 15 or more. In certain embodiments, a bispecific antibody molecule or antigen-binding fragment thereof described herein comprises one or more (G₄S)₂ linkers. In certain embodiments, a bispecific antibody molecule or antigen-binding fragment thereof described herein comprises one or more (G₄S)₃ linkers. In certain embodiments, a bispecific antibody molecule or antigen-binding fragment thereof described herein comprises one or more (G₄S)₄ linkers.

### Methods of Making

The present disclosure, among other things, provides methods of making bispecific antibody molecules or antigen-binding fragments thereof described herein. In some embodiments, an antibody or antigen-binding fragment thereof of a bispecific antibody molecule or antigen-binding fragment thereof described herein is identified using a display technology, such as yeast display, phage display, or ribosome display. In some embodiments, an antibody or antigen-binding fragment thereof of a bispecific antibody molecule or antigen-binding fragment thereof described herein is identified using a hybridoma library (e.g., a mammalian hybridoma library, e.g., a mouse hybridoma library), followed by supernatant screening.

Combinatorial methods for generating antibody molecules are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, each of which is hereby incorporated by reference in its entirety).

An antibody or antigen-binding fragment thereof of a bispecific antibody molecule or antigen-binding fragment thereof described herein can be derived from other species. A humanized antibody is an antibody produced by recombinant DNA technology, in which some or all amino acids of a human immunoglobulin light chain or heavy chain that are not required for antigen binding (e.g., constant regions and/or framework regions of variable domains) are used to substitute for the corresponding amino acids from light chain or heavy chain of the cognate, nonhuman antibody. By way of example, a humanized version of a murine antibody to a given antigen has on both heavy and light chains: (1) constant regions of a human antibody; (2) FRs from the variable domains of a human antibody; and (3) CDRs from the murine antibody. Human FRs may be selected based on their highest sequence homology to mouse FR sequence. When necessary, one or more residues in human FRs can be changed to residues at corresponding positions in a murine antibody so as to preserve binding affinity of the humanized antibody to a target. This change is sometimes called “back mutation.” Similarly, forward mutations may be made to revert back to murine sequence for a desired reason, e.g. stability or affinity to a target. Humanized antibodies generally are generally less likely to elicit an immune response in humans as compared to chimeric human antibodies because the former contain considerably fewer non-human components.

Methods for humanizing non-human antibodies are well known in the art. Suitable methods for making humanized antibodies in accordance with the present disclosure are described in, e.g., Winter EP 0 239 400; Jones et al., Nature 321:522-525 (1986); Riechmann et al., Nature 332:323-327 (1988); Verhoeyen et al., Science 239:1534-1536 (1988); Queen et al., Proc. Nat. Acad. ScL USA 86:10029 (1989); U.S. Pat. No. 6,180,370; and Orlandi et al., Proc. Natl. Acad. Sd. USA 86:3833 (1989); the disclosures of each of which are incorporated herein by reference in their entireties. Generally, transplantation of non-human (e.g., murine) CDRs onto a human antibody is achieved as follows. cDNAs encoding V_{H} and V_{L} are isolated from a hybridoma, and nucleic acid sequences encoding V_{H} and V_{L} including CDRs are determined by sequencing. Nucleic acid sequences encoding CDRs are inserted into corresponding regions of a human antibody V_{H} or V_{L} coding sequences and attached to human constant region gene segments of a desired isotype (e.g., yl for CH and K for CL). Humanized heavy and light chain genes are co-expressed in mammalian host cells (e.g., CHO or NSO cells) to produce soluble humanized antibody. To facilitate large-scale production of antibodies, it is often desirable to select for a high expressor using, for example, a DHFR gene or GS gene in the producer line.

An antibody molecule or antigen-binding fragment thereof of a bispecific antibody molecule or antigen-binding fragment thereof described herein can be or comprise a human antibody molecule or antigen-binding fragment thereof. Completely human antibodies may be particularly desirable for therapeutic treatment of human subjects. Human antibodies can be made by a variety of methods known in the art including phage display methods described above using antibody libraries derived from human immunoglobulin sequences (see, e.g., U.S. Pat. Nos. 4,444,887 and 4,716,111; and PCT publications WO 98/46645, WO 98/60433, WO 98/24893, WO 98/16664, WO 96/34096, WO 96/33735, and WO 91/10741; each of which is incorporated herein by reference in its entirety). Techniques are also available for the preparation of human monoclonal antibodies in, e.g., Cole et al., Monoclonal Antibodies and Cancer Therapy, Alan R. Riss, (1985); and Boerner et al., J. Immunol., 147 (1): 86-95, (1991), each of which is incorporated herein by reference in its entirety.

An antibody molecule or antigen-binding fragment thereof of a bispecific antibody molecule or antigen-binding fragment thereof described herein can be or comprise a chimeric antibody molecule or antigen-binding fragment thereof. Illustrative methods of making chimeric antibodies are described, for example, in U.S. Pat. No. 4,816,567; and Morrison et al., Proc. Natl. Acad. Sci. USA, 1984, 81:6851-6855; each of which is incorporated by reference in its entirety. In some embodiments, a chimeric antibody is made by using recombinant techniques to combine a non-human variable region (e.g., a variable region derived from a mouse, rat, hamster, rabbit, or non-human primate, such as a monkey) with a human constant region.

Any suitable method can be used to introduce variability into one or more polynucleotide sequences encoding a bispecific antibody molecule or antigen-binding fragment thereof described herein, including error-prone PCR, chain shuffling, and oligonucleotide-directed mutagenesis such as trinucleotide-directed mutagenesis (TRIM). In some embodiments, several CDR residues (e.g., 4-6 residues at a time) are randomized. CDR residues involved in antigen binding may be specifically identified, for example, using alanine scanning mutagenesis or modeling. CDR-H3 and CDR-L3 in particular are often targeted for mutation. Introduction of diversity into variable regions and/or CDRs can be used to produce a secondary library. A secondary library is then screened to identify antibody variants with improved affinity. Affinity maturation by constructing and reselecting from secondary libraries has been described, for example, in Hoogenboom et al., Methods in Molecular Biology, 2001, 178:1-37, incorporated by reference in its entirety.

### Nucleic Acids

The present disclosure, among other things, provides nucleic acids encoding bispecific antibody molecules or antigen-binding fragments thereof described herein. The present disclosure includes nucleic acids encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein. Such nucleic acids may be present in a vector. Such nucleic acids may be present in the genome of a cell, e.g., a cell of a subject in need of treatment or a cell for production of an antibody molecule or antigen-binding fragment thereof, e.g. a mammalian cell for production of at least one bispecific antibody molecule or antigen-binding fragment thereof described herein.

Nucleic acids encoding bispecific antibody molecules or antigen-binding fragment thereof described herein may be modified to include codons that are optimized for expression in a particular cell type or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acid encoding a bispecific antibody molecule or antigen-binding fragment thereof described herein, in whole or in part, may include an altered sequence to optimize codon usage for a particular cell type (e.g., a eukaryotic or prokaryotic cell). For example, a coding sequence for a humanized heavy (or light) chain variable region as described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector by methods known in the art, and nucleic acids may be operably linked to an expression control sequence. As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

A vector comprising any nucleic acids or fragments thereof described herein is further provided by the present disclosure. Any nucleic acids or fragments thereof described herein can be cloned into any suitable vector and can be used to transform or transfect any suitable host. Selection of vectors and methods to construct them are commonly known to persons of ordinary skill in the art (see, e.g., “Recombinant DNA Part D,” Methods in Enzymology, Vol. 153, Wu and Grossman, eds., Academic Press (1987)).

Conventionally used techniques including, for example, electrophoresis, calcium phosphate precipitation, DEAE-dextran transfection, or lipofection, may be used to introduce a foreign nucleic acid (e.g., DNA or RNA) into a prokaryotic or eukaryotic host cell. Desirably, a vector may include regulatory sequences, such as transcription and/or translation initiation and/or termination codons, which are specific to the type of host (e.g., bacterium, fungus, plant, or animal) into which a vector is to be introduced, as appropriate and taking into consideration whether a vector is DNA or RNA. In some embodiments, a vector comprises regulatory sequences that are specific to a genus of a host cell. In some embodiments, a vector comprises regulatory sequences that are specific to a species of a host.

In addition to a replication system and an inserted nucleic acid, a nucleic acid construct can include one or more marker genes, which allow for selection of transformed or transfected hosts. Exemplary marker genes include, e.g., biocide resistance (e.g., resistance to antibiotics or heavy metals) or complementation in an auxotrophic host to provide prototrophy.

An expression vector can comprise a native or nonnative promoter operably linked to an isolated or purified nucleic acid as described above. Selection of promoters, e.g., strong, weak, inducible, tissue-specific, and/or developmental-specific, is within the skill of one in the art. Similarly, combining a nucleic acid as described above with a promoter is also within the skill of one in the art.

Suitable vectors include those designed for propagation and expansion and/or for expression. For example, a cloning vector may be selected from the pUC series, the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). Bacteriophage vectors, such as λGT10, λGT11, λZapII (Stratagene), NEMBL4, and λNM1149, may be used. Examples of plant expression vectors that can be used include pBI110, pBI101.2, pBI101.3, pBI121, or pBIN19 (Clontech). Examples of animal expression vectors that can be used include pEUK-C1, pMAM, or pMAMneo (Clontech). The TOPO cloning system (Invitrogen, Carlsbad, Calif.) also can be used in accordance with the manufacturer's recommendations.

Additional sequences can be added to such cloning and/or expression sequences to optimize their function in cloning and/or expression, to aid in isolation of a nucleic acid encoding a bispecific antibody molecule or antigen-binding fragment thereof described herein, or to improve introduction of a nucleic acid into a cell. Use of cloning vectors, expression vectors, adapters, and linkers is well known in the art (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

In some embodiments, nucleic acids and vectors described herein are isolated and/or purified. Isolated nucleic acids and vectors may be prepared using standard techniques known in the art including, for example, alkali/SDS treatment, CsCl binding, column chromatography, and/or agarose gel electrophoresis. The composition can comprise other components as described further herein.

Any method known to one skilled in the art for insertion of nucleic acids into a vector may be used to construct expression vectors encoding a bispecific antibody molecule or antigen-binding fragment thereof described herein under control of transcriptional and/or translational control signals. These methods may include in vitro recombinant DNA and synthetic techniques and in vivo recombination (see, e.g., Ausubel, supra; or Sambrook, supra).

Antibodies that Bind to the Same Epitope

Bispecific antibody molecules or antigen-binding fragments thereof described herein include antibodies and antibody fragments that bind to one or more of the same epitopes as the antibody molecules or fragments thereof shown in Tables 1-5. As used herein, the term “epitope” refers to any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Additional antibody molecules or antigen-binding fragments can be identified based on their ability to cross-compete (e.g., to competitively inhibit binding of, in a statistically significant manner) with bispecific antibody molecules or antigen-binding fragments thereof described herein in TMIGD2 binding assays and/or TAA (e.g., HHLA2) binding assays. The ability of a test antibody molecule or antigen-binding fragment to inhibit binding of a bispecific antibody molecule or antigen-binding fragment thereof described herein to TMIGD2 demonstrates that the test antibody molecule or antigen-binding fragment can compete with that bispecific antibody molecule or antigen-binding fragment for binding to TMIGD2. Accordingly, such an antibody molecule or antigen-binding fragment may bind to the same or a related epitope on TMIGD2 as the bispecific antibody molecule or antigen-binding fragment thereof described herein. The ability of a test antibody molecule or antigen-binding fragment to inhibit binding of a bispecific antibody molecule or antigen-binding fragment thereof described herein to a TAA (e.g., HHLA2) demonstrates that the test antibody molecule or antigen-binding fragment can compete with that bispecific antibody molecule or antigen-binding fragment for binding to a TAA (e.g., HHLA2). Accordingly, such an antibody molecule or antigen-binding fragment may bind to the same or a related epitope on a TAA (e.g., HHLA2) as the bispecific antibody molecule or antigen-binding fragment thereof described herein.

### Additional Agents

The present disclosure, among other things, provides one or more additional agents (e.g., 2, 3, 4, 5, or more additional agents) that can be administered with bispecific antibody molecules or antigen-binding fragments thereof described herein in a combination therapy. As used herein, an additional agent may be or comprise any known treatment for a particular disease, disorder, or condition (e.g., a cancer). For example, an additional agent can be or comprise one or more of: a chemotherapeutic agent, a hormone therapy, a cell-based therapy, treatment with hyperthermia, a photodynamic therapy, surgery, radiation, or a transplant.

In some embodiments, a chemotherapeutic agent comprises or is one or more anthracyclines, one or more cytoskeletal disruptors (e.g. microtubule targeting agents such as taxanes, maytansine, and analogs thereof), one or more epothilones, one or more histone deacetylase inhibitors (HDACs), one or more topoisomerase inhibitors (e.g., one or more of inhibitors of topoisomerase I or topoisomerase II), one or more kinase inhibitors, one or more nucleotide analogs or nucleotide precursor analogs, one or more peptide antibiotics, one or more platinum-based agents, one or more retinoids, one or more vinca alkaloids, or a combination thereof.

In some embodiments, a chemotherapeutic agent comprises or is one or more of: Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fludarabine, Fluorouracil, Gemcitabine, Hydroxyurea, Idarubicin, Imatinib, Irinotecan, Maytansine and/or analogs thereof (e.g. DM1), Mechlorethamine, Mercaptopurine, Methotrexate, Mitoxantrone, a Maytansinoid, Oxaliplatin, Paclitaxel, Pemetrexed, Rituxan, Teniposide, Tioguanine, Topotecan, Valrubicin, Vinblastine, Vincristine, Vindesine, or Vinorelbine. In some embodiments, a chemotherapeutic agent comprises or is an antibody-drug conjugate (ADC). In some embodiments, an ADC comprises or is hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, lorvotuzumab mertansine, or a combination thereof.

In some embodiments, a hormone therapy may be or comprise tamoxifen, raloxifene, leuprolide, bicaluatmide, granisetron, flutamide, or a combination thereof. In some embodiments, a cell-based therapy comprises or is chimeric antigen receptor T (CAR-T) cells, CAR-NK cells, TCR-transduced T cells, dendritic cells, tumor infiltrating lymphocytes (TILs), natural killer (NK) cells, or a combination thereof. In some embodiments, treatment with hyperthermia comprises or is local hyperthermia (e.g., external, intraluminal, or interstitial hyperthermia), regional hyperthermia (e.g., deep tissue hyperthermia, regional perfusion, or (continuous hyperthermic peritoneal perfusion), or whole-body hyperthermia. In some embodiments, a photodynamic therapy comprises or is administration of photosensitizers, such as hematoporphyrin and its derivatives, Verteporfin (BPD-MA), phthalocyanine, photosensitizer Pc4, demethoxy-hypocrellin A, 2BA-2-DMHA, or a combination thereof. In some embodiments, surgery comprises or is surgery to remove cancerous or precancerous tissue. In some embodiments, a transplant comprises or is a stem cell transplant or an organ transplant.

In some embodiments, an additional agent is administered prior to, substantially simultaneously with, or subsequent to administration of a bispecific antibody molecule or antigen-binding fragment thereof described herein. In some embodiments, administration of a bispecific antibody molecule or antigen-binding fragment thereof described herein and an additional agent results in an improvement in a disease, disorder, or condition (e.g., cancer) to an extent that is greater than an improvement produced by either the bispecific antibody molecule or antigen-binding fragment thereof described herein or the additional agent alone. The difference between the combined effect and the effect of each agent alone can be a statistically significant difference. In some embodiments, the combined effect can be a synergistic effect. In some embodiments, combined administration of a bispecific antibody molecule or antigen-binding fragment thereof described herein and an additional agent allows administration of the additional agent at a reduced dose, a reduced number of doses, and/or a reduced frequency of dosage compared to a standard dosing regimen, e.g., an approved dosing regimen for the additional agent.

### Conjugates to Therapeutic Agents

The present disclosure, among other things, provides bispecific antibody molecules or antigen-binding fragments thereof conjugated to one or more therapeutic agents. In some embodiments, a therapeutic agent comprises or is a cytotoxic agent, a drug, and/or a radioisotope. When conjugated to a cytotoxic agent, such conjugates can be referred to as “immunotoxins.” A cytotoxic agent includes any agent that is detrimental to (e.g., can kill) cells. Examples of cytotoxic agents include, but are not limited to, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs of any of the foregoing.

Bispecific antibody molecules or antigen-binding fragments thereof described herein can be conjugated to one or more therapeutic agents (e.g., one or more drugs) including, but are not limited to, antimetabolites (e.g., methotrexate, 6-mercaptopurine, 6-thioguanine, cytarabine, and/or 5-fluorouracil decarbazine), alkylating agents (e.g., mechlorethamine, thiotepa chlorambucil, melphalan, carmustine (BSNU), lomustine (CCNU), cyclophosphamide, busulfan, dibromomannitol, streptozotocin, mitomycin C, and/or cis-dichlorodiamine platinum (II) (DDP) cisplatin), anthracyclines (e.g., daunorubicin (formerly daunomycin) and/or doxorubicin), antibiotics (e.g., dactinomycin, bleomycin, mithramycin, and/or anthramycin (AMC)), and anti-mitotic agents (e.g., vincristine and/or vinblastine). Bispecific antibody molecules or antigen-binding fragments thereof described herein can be conjugated to one or more radioisotopes (e.g., radioactive iodine) to generate cytotoxic radiopharmaceuticals for treating diseases, disorders, or conditions described herein, such as cancers described herein.

### Pharmaceutical Compositions

The present disclosure, among other things, provides pharmaceutical compositions comprising one or more bispecific antibody molecules or antigen-binding fragments described herein in combination with one or more pharmaceutically or physiologically acceptable carriers, diluents, or excipients.

When “a therapeutically effective amount” is indicated, a precise amount of a pharmaceutical composition comprising at least one bispecific antibody molecule or antigen-binding fragment thereof described herein can be determined by a physician with consideration of individual differences in age, weight, immune response, and condition of a subject.

Pharmaceutical compositions described herein may comprise buffers including, but not limited to, neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of at least one contaminant (e.g., an endotoxin).

Pharmaceutical compositions described herein may be administered in any manner appropriate to the disease, disorder, or condition to be treated or prevented. Quantity and frequency of administration will be determined by such factors as condition of a patient, and type and severity of a patient's disease, disorder, or condition, although appropriate dosages may be determined by clinical trials.

Pharmaceutical compositions described herein may be in a variety of forms. These include, for example, liquid, semi-solid, and solid dosage forms, such as liquid solutions (e.g., injectable and infusible solutions), dispersions or suspensions, liposomes, and suppositories. Preferred compositions may be injectable or infusible solutions. Pharmaceutical compositions described herein can be formulated for administration intravenously, subcutaneously, intradermally, intratumorally, intranodally, intramedullary, intramuscularly, transarterially, or intraperitoneally.

In some embodiments, a pharmaceutical composition described herein is formulated for parenteral (e.g., intravenous, subcutaneous, intraperitoneal, or intramuscular) administration. In some embodiments, a pharmaceutical composition described herein is formulated for subcutaneous, intravenous, intramuscular, or intrasternal injection or infusion. In preferred embodiments, a pharmaceutical composition described herein is formulated for subcutaneous or intravenous injection of infusion. Pharmaceutical compositions described herein can be formulated for administered by using infusion techniques that are commonly known in immunotherapy (see, e.g., Rosenberg et al., New Eng. J. of Med. 319:1676, 1988, which is hereby incorporated by reference in its entirety).

As used herein, the terms “parenteral administration” and “administered parenterally” refer to modes of administration other than enteral and topical administration, usually by injection or infusion, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoral, and intrasternal injection and infusion.

In some embodiments, pharmaceutical compositions described herein are administered in combination with (e.g., before, substantially simultaneously, or following) one or more additional agents. In some embodiments, an additional agent may be or comprise bone marrow transplantation or lymphocyte ablative therapy using one more chemotherapeutic agent (e.g., Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fludarabine, Fluorouracil, Gemcitabine, Hydroxyurea, Idarubicin, Imatinib, Irinotecan, Maytansine and/or analogs thereof (e.g. DM1), Mechlorethamine, Mercaptopurine, Methotrexate, Mitoxantrone, a Maytansinoid, Oxaliplatin, Paclitaxel, Pemetrexed, Rituxan, Teniposide, Tioguanine, Topotecan, Valrubicin, Vinblastine, Vincristine, Vindesine, Vinorelbine, or combinations thereof). In certain embodiments, subjects undergo standard treatment with high dose chemotherapy followed by peripheral blood stem cell transplantation. In certain embodiments, following transplant, subjects receive one or more pharmaceutical compositions described herein. In some embodiments, pharmaceutical compositions described herein are administered before or following surgery.

A dosage of any aforementioned therapy to be administered to a subject will vary with a disease, disorder, or condition being treated and based on a specific subject. Scaling of dosages for human administration can be performed according to art-accepted practices.

The present disclosure, among other things, provides pharmaceutical compositions comprising one or more bispecific antibody molecules or antigen-binding fragments thereof described herein and immune effector cells (e.g., T cells (e.g., naïve and/or effector T cells) and/or NK cells). In some embodiments, immune effector cells described herein are loaded with one or more bispecific antibody molecules or antigen-binding fragments thereof described herein. In some embodiments, immune effector cells described herein are pre-loaded with one or more bispecific antibody molecules or antigen-binding fragments thereof described herein prior to cryopreservation. In some embodiments, immune effector cells described herein are cryopreserved prior to loading with one or more bispecific antibody molecules or antigen-binding fragments thereof described herein. Pharmaceutical compositions comprising immune effector cells pre-loaded with one or more bispecific antibody molecules or antigen-binding fragments thereof, as described herein, may be administered to a patient suffering from a disease, disorder, or condition described herein. In some embodiments, immune effector cells comprise or are T cells (e.g., naïve and/or effector T cells). In some embodiments, immune effector cells comprise or are NK cells. In some embodiments, immune effector cells described herein are derived from a patient suffering from a disease, disorder, or condition described herein. In some embodiments, immune effector cells described herein are isolated from umbilical cord tissue, placental tissue, induced pluripotent stem cells (iPSCs), or peripheral blood mononuclear cells (PBMCs).

### Methods of Treatment

The present disclosure, among other things, provides methods of treating a disease, disorder or condition described herein in a subject comprising administering a pharmaceutical composition comprising one or more bispecific antibody molecules or antigen-binding fragments described herein. In some embodiments, a therapeutically effective amount of at least one pharmaceutical composition described herein is administered to a subject having a disease, disorder, or condition.

In some embodiments, the disclosure provides one or more bispecific antibody molecules or antigen-binding fragments described herein comprising amino acid sequences found in Table 1 for use as a medicament. In some embodiments, the disclosure provides one or more bispecific antibody molecules or antigen-binding fragments described herein comprising amino acid sequences found in Table 1 for use in the treatment of a disease, disorder, or condition described herein. In some embodiments, the disclosure provides use of one or more bispecific antibody molecules or antigen-binding fragments described herein comprising amino acid sequences found in Table 1 for the manufacture of a medicament for the treatment of a disease, disorder, or condition described herein.

Pharmaceutical compositions comprising one or more bispecific antibody molecules or antigen-binding fragments described herein (e.g., in Tables 1-5) can be for use in the manufacture of a medicament for treating a disease, disorder, or condition in a subject. Pharmaceutical compositions comprising one or more bispecific antibody molecules or antigen-binding fragments described herein can be for use in the manufacture of a medicament for modulating an immune response in a subject. Pharmaceutical compositions comprising one or more bispecific antibody molecules or antigen-binding fragments described herein can be administered to a subject in accordance with a dosage regimen described herein, alone or in combination with (e.g., before, substantially simultaneously, or following) one or more additional agents which may be or comprise one or more of therapeutic agents, procedures, or modalities.

A subject to be treated with methods described herein can be a mammal (e.g., a primate, mouse, humanized mouse, rat, non-human mammal, domestic animal, such as a dog, cat, cow, or horse), and is preferably a human (e.g., a patient having, or at risk of having, a disease, disorder or condition described herein). A subject can be an animal model of cancer, such as a xenograft animal model of a human-derived cancer. In some embodiments, a subject has not undergone treatment, such as chemotherapy, radiation therapy, targeted therapy, and/or immune checkpoint therapy. In another embodiment, a subject has undergone treatment, such as chemotherapy, radiation therapy, targeted therapy, and/or immune checkpoint therapy. In certain embodiments, a subject has had surgery to remove cancerous or precancerous tissue. In other embodiments, a cancerous tissue has not been removed, e.g., the cancerous tissue may be located in an inoperable region of the body, such as in a tissue that is essential for life or in a region where a surgical procedure would cause considerable risk of harm to a subject.

A method of treating (e.g., one or more of reducing, inhibiting, or delaying progression of) a cancer or a tumor in a subject with a pharmaceutical composition comprising one or more bispecific antibody molecules or antigen-binding fragments described herein is provided. A subject can have an adult or pediatric form of cancer. A cancer may be at an early, intermediate, or late stage, or a metastatic cancer.

Methods of treating (e.g., one or more of reducing, inhibiting, or delaying progression of) at least one sign or symptom of cancer in a subject with a pharmaceutical composition comprising one or more bispecific antibody molecules or antigen-binding fragments described herein are provided. In some embodiments, pharmaceutical compositions described herein are useful to delay the onset of, slow the progression of, or ameliorate one or more signs or symptoms of cancer. In some embodiments, a physiological sign or symptom of cancer comprises or is one or more of: an increase in tumor volume, an increase in number of cancer cells, an increase in number of metastases, a decrease in life expectancy, an increase in cancer cell proliferation, or an increase in cancer cell survival. In some embodiments, a physical sign or symptom of cancer comprises or is one or more of: a skin lesion (e.g., a lump or mole), weight loss, digestive problems, discomfort, fatigue, pain, trouble swallowing, cough, unusual bleeding and/or discharge, changes in bowel and/or bladder habits, or mental confusion.

A cancer can include, but is not limited to, a solid tumor, a hematological cancer (e.g., leukemia, lymphoma, or myeloma, e.g., multiple myeloma), a metastatic lesion, or a combination thereof. Examples of solid tumors include, but are not limited to, malignancies e.g., sarcomas and carcinomas, e.g., adenocarcinomas of the various organ systems, such as those affecting lungs, breast, ovaries, lymph nodes, genitals, gastrointestinal (e.g., colon) and genitourinary tracts (e.g., renal, urothelial, bladder cells, or prostate), pharynx, CNS (e.g., brain, neural, or glial cells), head and neck, skin (e.g., melanoma, e.g., a cutaneous melanoma), pancreas, and bones (e.g., a chordoma).

In some embodiments, a cancer comprises or is a lung cancer (e.g., a non-small cell lung cancer (NSCLC (e.g., a non-small cell lung cancer (NSCLC) with squamous and/or non-squamous histology or a NSCLC adenocarcinoma) or a small cell lung cancer (SCLC)), a skin cancer (e.g., a Merkel cell carcinoma or a melanoma (e.g., an advanced melanoma)), an ovarian cancer, a mesothelioma, a bladder cancer, a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS), a prostate cancer, a breast cancer (e.g., a breast cancer that does not express one, two or all of estrogen receptor, progesterone receptor, or Her2/neu, e.g., a triple negative breast cancer (TNBC)), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), a nasopharyngeal cancer, a duodenal cancer, an endometrial cancer, a pancreatic cancer, a head and neck cancer (e.g., head and neck squamous cell carcinoma (HNSCC)), an anal cancer, a gastro-esophageal cancer, a thyroid cancer (e.g., anaplastic thyroid carcinoma), a cervical cancer (e.g., a squamous cell carcinoma of the cervix), a neuroendocrine tumor (e.g., an atypical pulmonary carcinoid tumor), a lymphoproliferative disease (e.g., a post-transplant lymphoproliferative disease), a lymphoma (e.g., T-cell lymphoma, B-cell lymphoma, or a non-Hodgkin lymphoma), a myeloma (e.g., a multiple myeloma), a leukemia (e.g., a myeloid leukemia or a lymphoid leukemia), or a combination thereof.

In some embodiments, a cancer comprises or is a brain tumor, e.g., a glioblastoma, a gliosarcoma, or a recurrent brain tumor. In some embodiments, a cancer comprises or is a pancreatic cancer, e.g., an advanced pancreatic cancer. In some embodiments, a cancer comprises or is a skin cancer, e.g., a melanoma (e.g., a stage II-IV melanoma, an HLA-A2 positive melanoma, an unresectable melanoma, or a metastatic melanoma), or a Merkel cell carcinoma. In some embodiments, a cancer comprises or is a renal cancer, e.g., a renal cell carcinoma (RCC) (e.g., a metastatic renal cell carcinoma). In some embodiments, a cancer comprises or is a breast cancer, e.g., a metastatic breast carcinoma or a stage IV breast carcinoma, e.g., a TNBC. In some embodiments, a cancer comprises or is a virus-associated cancer. In some embodiments, a cancer comprises or is an anal canal cancer (e.g., a squamous cell carcinoma of an anal canal). In some embodiments, a cancer comprises or is a cervical cancer (e.g., a squamous cell carcinoma of the cervix). In some embodiments, a cancer comprises or is a gastric cancer (e.g., an Epstein Barr Virus (EBV) positive gastric cancer or a gastric or gastro-esophageal junction carcinoma). In some embodiments, a cancer comprises or is a head and neck cancer (e.g., an HPV-positive or negative squamous cell cancer of the head and neck (SCCHN)). In some embodiments, a cancer comprises or is a nasopharyngeal cancer (NPC). In some embodiments, a cancer comprises or is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer.

In some embodiments, a cancer comprises or is a hematological cancer. In some embodiments, a cancer comprises or is a leukemia, e.g., acute myeloid leukemia, chronic myeloid leukemia, acute lymphoblastic leukemia, chronic lymphocytic leukemia, chronic leukemia, or acute leukemia. In some embodiments, a cancer comprises or is a lymphoma, e.g., Hodgkin lymphoma (HL), non-Hodgkin's lymphoma, lymphocytic lymphoma, or DLBCL (e.g., a relapsed or refractory HL or DLBCL). In some embodiments, a cancer comprises or is a myeloma, e.g., multiple myeloma.

Administration of pharmaceutical compositions comprising one or more bispecific antibody molecules or antigen-binding fragments described herein may be carried out in any medically appropriate manner (e.g., injection, ingestion, transfusion, inhalation, implantation, or transplantation). In some embodiments, a pharmaceutical compositions described herein is administered by injection or infusion. Pharmaceutical compositions described herein may be administered to a patient transarterially, subcutaneously, intravenously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by subcutaneous, intravenous, intramuscular, or intrasternal infusion or injection. In some embodiments, a pharmaceutical composition described herein is administered by intramuscular or subcutaneous injection. Pharmaceutical compositions described herein may be injected directly into a site of inflammation, a local disease site, a lymph node, an organ, a tumor, or site of infection in a subject.

In some embodiments, one or more bispecific antibody molecules or antigen-binding fragments described herein is administered in combination with one or more other therapeutic agents or modalities. In some embodiments, one or more additional agents (e.g., therapeutic agents or modalities) comprises or is an anti-cancer agent or modality. In some embodiments, such a combination therapy shows a synergistic effect in treating cancer. Known additional agents (e.g., compounds or treatments that show therapeutic efficacy in treating cancer) may include, for example, one or more chemotherapeutic agents, alkylating agents, anti-metabolites, anti-microtubule agents, topoisomerase inhibitors, cytotoxic antibiotics, angiogenesis inhibitors, immunomodulators, vaccines, cell-based therapies (e.g. allogeneic or autologous stem cell transplantation), organ transplantation, radiation therapy, or surgery.

In some embodiments, a chemotherapeutic agent may be or comprise one or more anthracyclines, one or more cytoskeletal disruptors (e.g. microtubule targeting agents such as taxanes, maytansine, and analogs thereof), one or more epothilones, one or more histone deacetylase inhibitors (HDACs), one or more topoisomerase inhibitors (e.g., one or more of inhibitors of topoisomerase I or topoisomerase II), one or more kinase inhibitors, one or more nucleotide analogs or nucleotide precursor analogs, one or more peptide antibiotics, one or more platinum-based agents, one or more retinoids, one or more vinca alkaloids, or a combination thereof. In some embodiments, a chemotherapeutic agent may be or comprise one or more of: Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fludarabine, Fluorouracil, Gemcitabine, Hydroxyurea, Idarubicin, Imatinib, Irinotecan, Maytansine and/or analogs thereof (e.g. DM1), Mechlorethamine, Mercaptopurine, Methotrexate, Mitoxantrone, a Maytansinoid, Oxaliplatin, Paclitaxel, Pemetrexed, Rituxan, Teniposide, Tioguanine, Topotecan, Valrubicin, Vinblastine, Vincristine, Vindesine, or Vinorelbine. In some embodiments, a chemotherapeutic agent may be an antibody-drug conjugate (ADC). In some embodiments, an ADC comprises or is hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, lorvotuzumab mertansine, or a combination thereof.

### Kits

The present disclosure, among other things, provides kits comprising one or more bispecific antibody molecules or antigen-binding fragments described herein, and instructions for use and/or administration. In some embodiments, a kit comprises one or more bispecific antibody molecules or antigen-binding fragments described herein and a pharmaceutically acceptable carrier, and instructions for use and/or administration.

In some embodiments, a kit comprises instructions for use in any method described herein. Instructions can comprise a description of administration of a first pharmaceutical composition and second composition (e.g., and additional agent) to a subject to achieve an intended activity in a subject. The kit may further comprise a description of selecting a subject suitable for treatment based on identifying whether a subject is in need of treatment. In some embodiments, instructions comprise a description of administering a first pharmaceutical composition and second pharmaceutical composition to a subject who is in need of treatment.

Instructions relating to a first pharmaceutical composition and second composition (e.g., an additional agent) described herein generally include information as to dosage, dosing schedule, and route of administration for the intended treatment. Containers may be unit doses, bulk packages (e.g., multi-dose packages), or sub-unit doses. Instructions supplied in kits described herein are typically written instructions on a label or package insert. A label or package insert indicates that one or more pharmaceutical compositions are used for treating, delaying onset, and/or alleviating a disease, disorder or condition in a subject.

Kits provided herein are in suitable packaging. Suitable packaging includes, but is not limited to, vials, bottles, jars, and/or flexible packaging. Also contemplated are packages for use in combination with a specific device, such as an infusion device. A kit may have a sterile access port (for example, a container may be an intravenous solution bag or a vial having a stopper pierce able by a hypodermic injection needle). A container may also have a sterile access port.

Kits optionally may provide additional components, such as buffers and/or interpretive information. Normally, a kit comprises a container and a label or package insert(s) on or associated with a container. In some embodiment, the disclosure provides articles of manufacture comprising contents of kits described above.

### INCORPORATION BY REFERENCE

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

The disclosure is further illustrated by the following Examples. Examples are provided for illustrative purposes only. It is not to be construed as limiting the scope or content of the disclosure in any way.

### EXAMPLES

The following Examples are provided so as to describe to the skilled artisan how to make and use methods and compositions described herein, and are not intended to limit the scope of the present disclosure.

### Example 1: Characterization of Certain TMIGD2-TAA Bispecific Antibody Molecules

The present Example demonstrates methods for characterizing bispecific antibody molecules or antigen-binding fragments thereof, specifically an anti-TMIGD2 and anti-tumor-associated antigen (TAA) bispecific antibody molecule or antigen-binding fragment thereof described herein. The present Example further provides various methods for determining and/or characterizing relevant functional activity of bispecific antibody molecules or antigen-binding fragments thereof described herein.

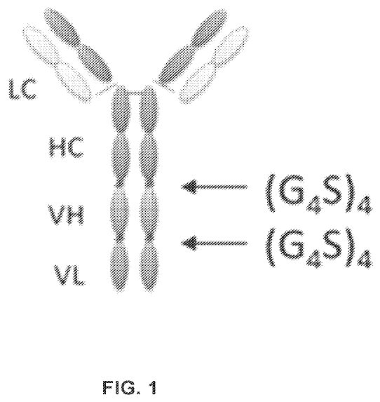

A schematic of an exemplary bispecific antibody molecule including a scFv targeting TMIGD2 attached to a C-terminus of a heavy chain of an IgG antibody targeting a TAA (tetravalent Morrison bispecific format) is shown in FIG. 1. L234A/L235A mutations were introduced in an Fc region to reduce effector function and prevent destruction of TMIGD2-expressing immune cells in circulation. An internal linker within a scFv domain (between V_{H} and V_{L} domains) can include or be a (G₄S)₄ repeat. Linkers between Fc tail and scFv domain can include or be (G₄S)₂, (G₄S)₃, or (G₄S)₄ repeats.

An exemplary bispecific antibody molecule including a scFv targeting TMIGD2 (Amplimmune 4.5 (Amp4.5) as described in WO2014100823A1, which is hereby incorporated by reference in its entirety) attached to a C-terminus of a heavy chain of an IgG antibody targeting CD20 (Rituximab) was constructed and used in assays described herein. Additional exemplary anti-TMIGD2/anti-CD20 bispecific antibodies (TK20-17C7-01, TK20-29F8-16, and TK20-39C9-2134) were also constructed and used in assays herein.

### Jurkat Reporter Assay

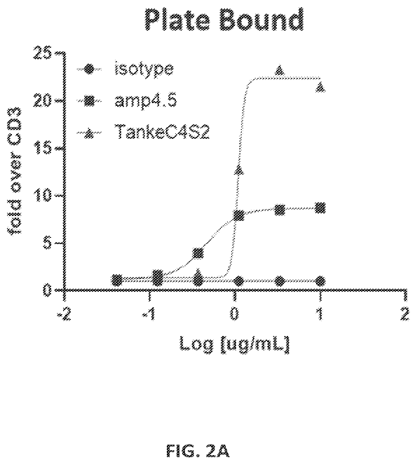

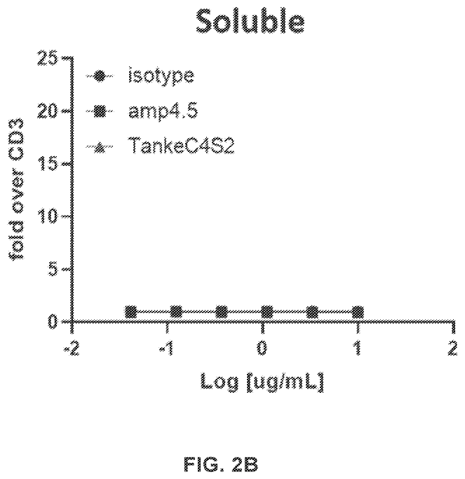

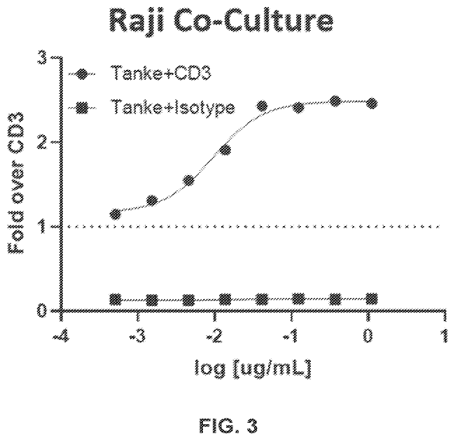

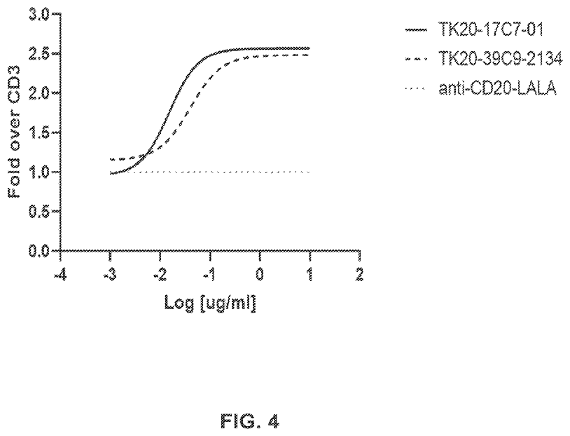

To assess enhancement of TCR stimulation by the exemplary anti-TMIGD2/anti-CD20 antibody molecule, TMIGD2 was overexpressed in Jurkat cells expressing an NFAT-driven luciferase reporter. Engagement of TMIGD2 on these cells with an anti-TMIGD2/anti-CD20 antibody molecule provided herein increased T cell activation above anti-CD3 stimulation alone when the bispecific antibody molecule was immobilized on plastic (FIG. 2A) or when bound to CD20+ Raji cells (FIG. 3). These results were corroborated with additional anti-TMIGD2/anti-CD20 antibody molecules provided herein (TK20-17C7-01, TK20-29F8-16, and TK20-39C9-2134), confirming that such anti-TMIGD2/anti-CD20 antibody molecules increased T cell activation when bound to CD20+ Raji cells (FIG. 4).

Enhancement of T cell activation induced by the anti-TMIGD2/anti-CD20 bispecific antibody molecule was superior to that observed with a human IgG4 version of Amp4.5 (FIG. 2A). No enhancement of T cell signaling was observed when the anti-TMIGD2/anti-CD20 bispecific antibody molecule were added in solution (FIG. 2B) and plate bound anti-TMIGD2/anti-CD20 bispecific antibody molecule did not induce T cell activation in the absence of anti-CD3 antibody. These data show that the TMIGD2/anti-CD20 bispecific antibody molecule displayed agonist activity and enhanced T cell activation in a TMIGD2-jurkat reporter assay, including in the presence of TAA (CD20)-expressing Raji target cells.

### Plate Bound Assay

Anti-TMIGD2/anti-CD20 bispecific antibody molecule, anti-TMIGD2 IgG4 (Amp4.5), or human IgG4 isotype control were coated onto wells of a 96-well white flat bottom tissue culture plate at 10 ug/mL with 0.5 ug/mL anti-CD3 antibody (OKT3) in 50 ul PBS overnight at 4° C. Plates were washed 3 times with PBS and 4×10⁴ TMIGD2-Jurkat NFAT reporter cells were added to each well in 100 μl complete RPMI containing 10% FBS. In some wells, anti-TMIGD2/anti-CD20 bispecific antibody molecule were added at 10 μg/mL in culture medium. Cells were incubated at 37° C. for 6 hours and 100 μl of Bio-Glo™ luciferase assay reagent (Promega®, G7940) was added to each well. Luciferase activity was measured on a plate reader.

### Raji Co-Culture Assay

Raji cells (2×10⁴) were plated in a 96-well white flat bottom tissue culture plate with 10 μg/mL anti-TMIGD2/anti-CD20 bispecific antibody molecule in 50 μl complete RPMI containing 10% FBS and incubated at 37° C. for 30 minutes. TMIGD2-Jurkat NFAT reporter cells (4×10⁴) were added to each well with 1 μg/mL anti-CD3 antibody (OKT3) or moue IgG2a isotype control in 50 μl complete RPMI. Cells were incubated at 37° C. for 6 hours and 100 ul of Bio-Glo™ luciferase assay reagent (Promega®, G7940) was added to each well. Luciferase activity was measured on a plate reader.

Raji cells (2×10⁴) were plated in a 96-well white flat bottom tissue culture plate with anti-TMIGD2/anti-CD20 bispecific antibodies (TK20-17C7-01, TK20-29F8-16, and TK20-39C9-2134) in 50 μl R10 medium (RPMI 1640+10% Fetal bovine serum) at 3-fold serial dilutions ranging from 10-0.5 μg/mL. An anti-CD20 (IgG1 LALA) was included as a negative control. The cells were incubated at 37° C. for 30 minutes. TMIGD2-Jurkat NFAT reporter cells (4×10⁴) were added to each well with 1 μg/mL anti-CD3 antibody (OKT3) in 50 μl R10 medium. Cells were incubated at 37° C. for 6 hours and 100 ul of Bio-Glo™ luciferase assay reagent (Promega®, G7940) was added to each well. Luciferase activity was measured on a plate reader.

### Example 2: Characterization of Certain TMIGD2-TAA Bispecific Antibody Molecules

The present Example demonstrates methods for characterizing bispecific antibody molecules or antigen-binding fragments thereof, specifically anti-TMIGD2 and anti-TAA (e.g., HHLA2) bispecific antibody molecules or antigen-binding fragments thereof described herein. The present Example further provides various methods for determining and/or characterizing relevant functional activity of bispecific antibody molecules or antigen-binding fragments thereof described herein. An exemplary bispecific antibody molecule including a scFv targeting TMIGD2 attached to a C-terminus of a heavy chain of an IgG antibody targeting HHLA2 will be constructed and used in assays described herein (see, e.g., Tables 1-4).

### Jurkat Reporter Assay

To assess enhancement of TCR stimulation by the exemplary anti-TMIGD2/anti-HHLA2 antibody molecule, TMIGD2 will be overexpressed in Jurkat cells expressing an NFAT-driven luciferase reporter. Engagement of TMIGD2 on these cells with the anti-TMIGD2/anti-HHLA2 antibody molecule will be assessed in comparison to anti-CD3 antibody stimulation alone with the TMIGD2/anti-HHLA2 bispecific antibody molecule immobilized on plastic, in solution, or in the presence of HHLA2-expressing Raji cells.

### Plate Bound Assay

Anti-TMIGD2/anti-HHLA2 antibody molecule, anti-TMIGD2 IgG, or human IgG1 with Fc silencing mutations isotype control will be coated onto wells of a 96-well white flat bottom tissue culture plate at about 10 ug/mL with about 0.5 ug/mL anti-CD3 antibody (OKT3) in about 50 ul PBS overnight at 4° C. Plates will be washed 3 times with PBS and about 4×10⁴ TMIGD2-Jurkat NFAT reporter cells will be added to each well in about 100 μl complete RPMI containing about 10% FBS. In some wells, anti-TMIGD2/anti-HHLA2 bispecific antibody molecule will be added at about 10 g/mL in culture medium. Cells will be incubated at about 37° C. for about 6 hours and about 100 μl of Bio-Glo™ luciferase assay reagent (Promega®, G7940) will be added to each well. Luciferase activity will be measured on a plate reader.

### Raji Co-Culture Assay

Raji cells (2×10⁴) will be plated in a 96-well white flat bottom tissue culture plate with about 10 μg/mL anti-TMIGD2/anti-HHLA2 bispecific antibody molecule in about 50 μl complete RPMI containing about 10% FBS and incubated at about 37° C. for about 30 minutes. TMIGD2-Jurkat NFAT reporter cells (about 4×10⁴) will be added to each well with about 1 μg/mL anti-CD3 (OKT3) or moue IgG isotype control in about 50 μl complete RPMI. Cells will be incubated at about 37° C. for about 6 hours and about 100 ul of Bio-Glo™ luciferase assay reagent (Promega®, G7940) will be added to each well. Luciferase activity will be measured on a plate reader.

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the disclosure. Accordingly, the foregoing description and drawing are by way of example only and any invention described in the present disclosure if further described in detail by the claims that follow.

Those skilled in the art will appreciate typical standards of deviation or error attributable to values obtained in assays or other processes described herein. The publications, websites and other reference materials referenced herein to describe the background of the disclosure and to provide additional detail regarding its practice are hereby incorporated by reference in their entireties.

1. A bispecific antibody molecule or antigen-binding fragment thereof comprising:
(i) a first antigen-binding domain that selectively binds to transmembrane and immunoglobulin domain containing 2 (TMIGD2); and
(ii) a second antigen-binding domain that selectively binds to at least one tumor-associated antigen (TAA).

2. The bispecific antibody molecule or antigen-binding fragment thereof of claim 1,
wherein the first antigen-binding domain activates immune effector cells.

3. The bispecific antibody molecule or antigen-binding fragment thereof of claim 1 or 2, wherein the first antigen-binding domain recruits immune effector cells to a tumor microenvironment.

4. The bispecific antibody molecule or antigen-binding fragment thereof of claim 2 or 3, wherein the immune effector cells comprise or are T cells and/or NK cells.

5. The bispecific antibody molecule or antigen-binding fragment thereof of any one of claims 1-4, wherein the at least one TAA comprises or is HHLA2, CD123, B7-H3, B7-H4, CD33, CD30, HER2, CD22, CD79b, Nectin-4, Trop-2, BCMA, CD19, folate receptor a, EGFR, or combinations thereof.

6. The bispecific antibody molecule or antigen-binding fragment thereof of any one of claims 1-5, wherein the first antigen-binding domain comprises or is an antibody or an antigen-binding fragment thereof.

7. The bispecific antibody molecule or antigen-binding fragment thereof of any one of claims 1-6, wherein the second antigen-binding domain comprises or is an antibody or an antigen-binding fragment thereof.

8. The bispecific antibody molecule or antigen-binding fragment thereof of claim 6 or 7, wherein the antigen-binding fragment comprises or is an scFv, Fab, Fab′, F(ab′)₂, nanobody, or camelid antibody.

9. The bispecific antibody molecule or antigen-binding fragment thereof of any one of claims 6-8, wherein the antibody or antigen-binding fragment thereof is or comprises:
(i) a chimeric, human, or humanized antibody molecule or antigen-binding fragment thereof; and/or
(ii) a monoclonal antibody molecule or antigen-binding fragment thereof.

10. The bispecific antibody molecule or antigen-binding fragment thereof of any one of claims 6-9, wherein the antibody or antigen-binding fragment thereof is or comprises:
(i) a heavy chain constant region chosen from IgG1, IgG2, IgG3, or IgG4, and/or
(ii) a light chain constant region chosen from light chain constant regions of kappa or lambda.

11. The bispecific antibody molecule or antigen-binding fragment thereof of any one of claims 6-9, wherein the antibody or antigen-binding fragment comprises an Fc region with reduced effector function relative to a reference antibody or antigen-binding fragment thereof.

12. The bispecific antibody molecule or antigen-binding fragment thereof of claim 11, wherein the Fc region comprises one or more amino acid substitutions of L234A and/or L235A, numbering according to EU index.

13. The bispecific antibody molecule or antigen-binding fragment thereof of any one of the preceding claims, wherein the first antigen-binding domain comprises or is a scFv and the second antigen-binding domain comprises or is an antibody molecule.

14. The bispecific antibody molecule or antigen-binding fragment thereof of claim 13, wherein the scFv and the antibody molecule are covalently linked by a peptide linker.

15. A pharmaceutical composition comprising at least one bispecific antibody molecule or antigen-binding fragment thereof of any one of claims 1-14, and a pharmaceutically acceptable carrier.

16. A method of treating a subject having a disease, disorder, or condition and/or modulating an immune response in a subject comprising administering a therapeutically effective amount of a pharmaceutical composition of claim 15.

17. The method of claim 15 or 16, wherein the subject has or is at risk of developing a cancer.

18. The method of claim 17, wherein the subject has a solid tumor or a hematological cancer.

19. The method of claim 18, wherein the solid tumor is or comprises one or more of: a renal cancer, a bone cancer, a skin cancer, a breast cancer, a cervical cancer, a colorectal cancer, an endometrial cancer, a lung cancer, an ovarian cancer, a liver cancer, cholangiocarcinoma, or a thyroid cancer.

20. The method of claim 18, wherein the hematological cancer comprises or is a leukemia or lymphoma.

21. The method of claim 20, wherein the leukemia comprises or is acute lymphocytic leukemia, acute myeloid leukemia, chronic myeloid leukemia, acute lymphoblastic leukemia, chronic lymphocytic leukemia, chronic leukemia, or acute leukemia.

22. The method of claim 20, wherein the lymphoma comprises or is Hodgkin lymphoma (HL), non-Hodgkin's lymphoma, lymphocytic lymphoma, or diffuse large B cell lymphoma (DLBCL).

23. The method of any one of claims 16-22, wherein the bispecific antibody molecule or antigen-binding fragment thereof is administered in combination with at least one additional agent.

24. A nucleic acid molecule encoding a bispecific antibody molecule or antigen-binding fragment thereof of any one of claims 1-14.

25. An expression vector comprising the nucleic acid molecule of claim 24.

26. A host cell comprising or expressing the bispecific antibody molecule or antigen-binding fragment thereof of any one of claims 1-14, the nucleic acid molecule of claim 24, or the expression vector of claim 25.

27. A method of making a bispecific antibody molecule or antigen-binding fragment thereof, comprising:
(i) culturing a host cell comprising the nucleic acid molecule of claim 24 or the expression vector of claim 25 under conditions suitable for expression of the bispecific antibody molecule or antigen-binding fragment thereof, and
(ii) recovering the bispecific antibody molecule or antigen-binding fragment thereof.

28. A method of detecting the presence or level of a TMIGD2 polypeptide and/or a TAA in a sample comprising detecting the TMIGD2 polypeptide and/or the TAA in the sample using the bispecific antibody molecule or antigen-binding fragment thereof of any one of claims 1-14.

29. A kit comprising at least one bispecific antibody molecule or antigen-binding fragment thereof of any one of claims 1-14, and instructions for use.
